(19) United States
(12) Patent Application Publication
(10) Pub. No.: US 2012/0101124 A1
(43) Pub. Date: Apr. 26, 2012

(54) 1,2,4-OXADIAZOL DERIVATIVES, THEIR PHARMACEUTICAL COMPOSITIONS AND THEIR USE AS SPHINGOSINE 1-PHOSPHATE 1 RECEPTOR AGONISTS

(75) Inventors: Xichen Lin, Shanghai (CN); Feng Ren, Shanghai (CN); Yugui Si, Shanghai (CN)

(73) Assignee: GLAXO GROUP LIMITED, Greenford, Middlesex (GB)

(21) Appl. No.: 13/379,214

(22) PCT Filed: Jun. 17, 2010
(86) PCT No.: PCT/CN2010/000869

§ 371 Date: Dec. 19, 2011

(30) Foreign Application Priority Data
Jun. 19, 2009 (GB) 0910667.5

(51) Int. Cl. A61K 31/437; A61K 31/4439; C07D 413/14; C07D 413/10; A61K 31/4245; A61P 25/00; A61P 29/00; A61P 11/06; A61P 19/02; A61P 1/00; A61P 17/06; A61P 35/00; A61P 35/04; A61P 31/12; A61P 3/10; A61P 9/00; C07D 471/04

(52) U.S. Cl. 514/300; 546/113; 514/337; 546/269.4; 548/131; 514/364

(57) ABSTRACT

1,2,4-Oxadiazol derivatives represented by formula (I) useful as sphingosine 1-phosphate 1 (S1P1) receptor agonists, processes for their preparation, pharmaceutical compositions containing them and their use in the treatment of various disorders mediated via S1P1 receptor are disclosed.

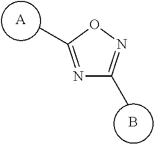

The present invention relates to novel compounds having pharmacological activity, processes for their preparation, pharmaceutical compositions containing them and their use in the treatment of various disorders.

Sphingosine 1-phosphate (S1P) is a bioactive lipid mediator formed by the phosphorylation of sphingosine by sphingosine kinases and is found in high levels in the blood. It is produced and secreted by a number of cell types, including those of hematopoietic origin such as platelets and mast cells (Okamoto et al 1998 J Biol Chem 273(42):27104; Sanchez and Hla 2004, J Cell Biochem 92:913). It has a wide range of biological actions, including regulation of cell proliferation, differentiation, motility, vascularisation, and activation of inflammatory cells and platelets (Pyne and Pyne 2000, Biochem J. 349: 385). Five subtypes of S1P responsive receptor have been described, S1P1 (Edg-1), S1P2 (Edg-5), S1P3 (Edg-3), S1P4 (Edg-6), and S1P5 (Edg-8), forming part of the G-protein coupled endothelial differentiation gene family of receptors (Chun et al 2002 Pharmacological Reviews 54:265, Sanchez and Hla 2004 J Cellular Biochemistry, 92:913). These 5 receptors show differential mRNA expression, with S1P1-3 being widely expressed, S1P4 expressed on lymphoid and hematopoietic tissues and S1P5 primarily in brain and to a lower degree in spleen. They signal via different subsets of G proteins to promote a variety of biological responses (Kluk and Hla 2002 Biochem et Biophysica Acta 1582:72, Sanchez and Hla 2004, J Cellular Biochem 92:913).

Proposed roles for the S1P1 receptor include lymphocyte trafficking, cytokine induction/suppression and effects on endothelial cells (Rosen and Goetzl 2005 Nat Rev Immunol. 5:560). Agonists of the S1P1 receptor have been used in a number of autoimmune and transplantation animal models, including Experimental Autoimmune Encephalomelitis (EAE) models of MS, to reduce the severity of the induced disease (Brinkman et al 2003 JBC 277:21453; Fujino et al 2003 J Pharmacol Exp Ther 305:70; Webb et al 2004 J Neuroimmunol 153:108; Rausch et al 2004 J Magn Reson Imaging 20:16). This activity is reported to be mediated by the effect of S1P1 agonists on lymphocyte circulation through the lymph system. Treatment with S1P1 agonists results in the sequestration of lymphocytes within secondary lymphoid organs such as the lymph nodes, inducing a reversible peripheral lymphopoenia in animal models (Chiba et al 1998, J Immunology 160:5037, Forrest et al 2004 J Pharmacol Exp Ther 309:758; Sanna et al 2004 JBC 279:13839). Published data on agonists suggests that compound treatment induces loss of the S1P1 receptor from the cell surface via internalisation (Graler and Goetzl 2004 FASEB J 18:551; Matloubian et al 2004 Nature 427:355; Jo et al 2005 Chem Biol 12:703) and it is this reduction of S1P1 receptor on immune cells which contributes to the reduction of movement of T cells from the lymph nodes back into the blood stream.

S1P1 gene deletion causes embryonic lethality. Experiments to examine the role of the S1P1 receptor in lymphocyte migration and trafficking have included the adoptive transfer of labelled S1P1 deficient T cells into irradiated wild type mice. These cells showed a reduced egress from secondary lymphoid organs (Matloubian et al 2004 Nature 427:355).

S1P1 has also been ascribed a role in endothelial cell junction modulation (Allende et al 2003 102:3665, Blood Singelton et al 2005 FASEB J 19:1646). With respect to this endothelial action, S1P1 agonists have been reported to have an effect on isolated lymph nodes which may be contributing to a role in modulating immune disorders. S1P1 agonists caused a closing of the endothelial stromal ‘gates’ of lymphatic sinuses which drain the lymph nodes and prevent lymphocyte egress (Wei wt al 2005, Nat. Immunology 6:1228).

The immunosuppressive compound FTY720 (JP11080026-A) has been shown to reduce circulating lymphocytes in animals and man, have disease modulating activity in animal models of immune disorders and reduce remission rates in relapsing remitting Multiple Sclerosis (Brinkman et al 2002 JBC 277:21453, Mandala et al 2002 Science 296:346, Fujino et al 2003 J Pharmacology and Experimental Therapeutics 305:45658, Brinkman et al 2004 American J Transplantation 4:1019, Webb et al 2004 J Neuroimmunology 153:108, Morris et al 2005 Eur J Immunol 35:3570, Chiba 2005 Pharmacology and Therapeutics 108:308, Kahan et al 2003, Transplantation 76:1079, Kappos et al 2006 New Eng J Medicine 335:1124). This compound is a prodrug that is phosphorylated in vivo by sphingosine kinases to give a molecule that has agonist activity at the S1P1, S1P3, S1P4 and S1P5 receptors. Clinical studies have demonstrated that treatment with FTY720 results in bradycardia in the first 24 hours of treatment (Kappos et al 2006 New Eng J Medicine 335:1124). The bradycardia is thought to be due to agonism at the S1P3 receptor, based on a number of cell based and animal experiments. These include the use of S1P3 knock-out animals which, unlike wild type mice, do not demonstrate bradycardia following FTY720 administration and the use of S1P1 selective compounds. (Hale et al 2004 Bioorganic & Medicinal Chemistry Letters 14:3501, Sanna et al 2004 JBC 279:13839, Koyrakh et al 2005 American J Transplantation 5:529)

Hence, there is a need for S1P1 receptor agonist compounds with selectivity over S1P3 which might be expected to show a reduced tendency to induce bradycardia.

The following patent applications describe oxadiazole derivatives as S1P1 agonists: WO03/105771, WO05/058848, WO06/047195, WO06/100633, WO06/115188, WO06/131336, WO07/024922 and WO07/116866.

The following patent application describes indole-oxadiazole derivatives as antipicornaviral agents: WO96/009822. The following patent applications describe indole-carboxylic acid derivatives as leukotriene receptor antagonists, pesticides and agrochemical fungicides respectively: WO06/090817, EP 0 439 785 and DE 39 39 238.

International patent applications WO08/074821 and WO08/76356 describe oxadiazole-indole derivatives as S1P1 agonists.

A structurally novel class of compounds has now been found which provides agonists of the S1P1 receptor.

The present invention therefore provides compounds of formula (I) or a salt thereof:

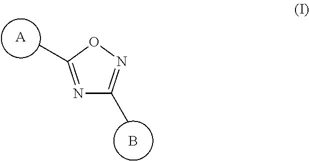

wherein

A is an aromatic ring selected from:

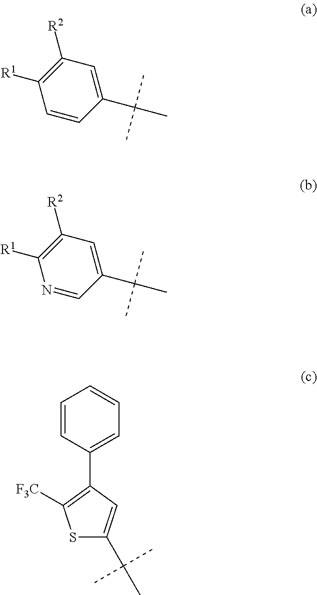

R¹ is C₍₁₋₆₎alkoxy or C₍₁₋₆₎alkyl;

R² is halogen, cyano or CF₃;

B is a bicyclic ring selected from:

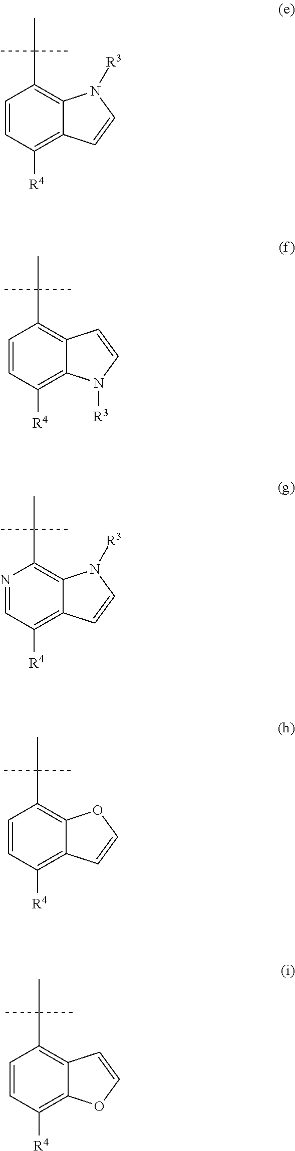

R³ is hydrogen or C₍₁₋₃₎alkyl; and

R⁴ is C₍₀₋₆₎alkyl-COOH optionally optionally interrupted by a cyclopropyl, piperidinyl, azetidinyl, pyrrolidinyl, optionally interrupted by N or O and optionally substituted by O, cyclopropyl, halogen or methyl.

In one embodiment of the invention R¹ is C₍₁₋₃₎alkoxy. In another embodiment R¹ is isopropoxy.

In one embodiment R² is chloro or cyano.

In one embodiment of the invention A is (a) or (b).

In one embodiment R³ is hydrogen, methyl or ethyl.

In one embodiment of the invention, when B is (e), (f) or (g), R⁴ is C₍₀₋₃₎alkyl-COOH.

In one embodiment, when B is (h) or (i), R⁴ is C₍₀₋₆₎alkyl-COOH or O—C₍₁₋₃₎alkyl-COOH.

In another embodiment, when B is (h) or (i), R⁴ is C₍₂₎alkyl-COOH or O—C₍₁₋₃₎alkyl-COOH.

In one embodiment of the invention

A is (a) or (b)

R¹ is C₍₁₋₃₎alkoxy;

R² is chloro or cyano;

R³ is hydrogen, methyl or ethyl; and

when B is (e), (f) or (g), R⁴ is C₍₀₋₃₎alkyl-COON

In one embodiment of the invention

A is (a) or (b);

R¹ is isopropoxy;

R² is chloro or cyano;

R³ is hydrogen, methyl or ethyl; and

when B is (e), (f) or (g), R⁴ is C₍₀₋₃₎alkyl-COON

In one embodiment of the invention

A is (a) or (b);

R¹ is C₍₁₋₃₎alkoxy;

R² is chloro or cyano;

R³ is hydrogen, methyl or ethyl; and

when B is (h) or (i), R⁴ is O—C₍₁₋₃₎alkyl-COOH.

In one embodiment of the invention

A is (a) or (b);

R¹ is isopropoxy;

R² is chloro or cyano;

R³ is hydrogen, methyl or ethyl; and

when B is (h) or (i), R⁴ is O—C₍₁₋₃₎alkyl-COON.

The term “alkyl” as a group or part of a group e.g. alkoxy or hydroxyalkyl refers to a straight or branched alkyl group in all isomeric forms. The term “C₍₁₋₆₎ alkyl” refers to an alkyl group, as defined above, containing at least 1, and at most 6 carbon atoms Examples of such alkyl groups include methyl, ethyl, propyl, iso-propyl, n-butyl, iso-butyl, sec-butyl, or tent-butyl. Examples of such alkoxy groups include methoxy, ethoxy, propoxy, iso-propoxy, butoxy, iso-butoxy, sec-butoxy and tert-butoxy.

As used herein, the term “halogen” refers to fluorine (F), chlorine (Cl), bromine (Br), or iodine (I) and the term “halo” refers to the halogen: fluoro (—F), chloro (—Cl), bromo(—Br) and iodo(—I).

The term “heteroaryl” represents an unsaturated ring which comprises one or more heteroatoms selected from O, N or S. Examples of 5 or 6 membered heteroaryl rings include pyrrolyl, triazolyl, thiadiazolyl, tetrazolyl, imidazolyl, pyrazolyl, isothiazolyl, thiazolyl, isoxazolyl, oxazolyl, oxadiazolyl, furazanyl, furanyl, thienyl, pyridyl, pyrimidinyl, pyrazinyl, pyridazinyl and triazinyl.

In certain of the compounds of formula (I), dependent upon the nature of the substituent there are chiral carbon atoms and therefore compounds of formula (I) may exist as stereoisomers. The invention extends to all optical isomers such as stereoisomeric forms of the compounds of formula (I) including enantiomers, diastereoisomers and mixtures thereof, such as racemates. The different stereoisomeric forms may be separated or resolved one from the other by conventional methods or any given isomer may be obtained by conventional stereoselective or asymmetric syntheses.

Certain of the compounds herein can exist in various tautomeric forms and it is to be understood that the invention encompasses all such tautomeric forms.

Suitable compounds of formula (I) are:

3-[7-(5-{3-Chloro-4-[(1-methylethyl)oxy]phenyl}-1,2,4-oxadiazol-3-yl)-1H-indol-4-yl]propanoic acid

3-[7-(5-{3-Chloro-4-[(1-methylethyl)oxy]phenyl}-1,2,4-oxadiazol-3-yl)-1-methyl-1H-indol-4-yl]propanoic acid

3-[7-(5-{3-Chloro-4-[(1-methylethyl)oxy]phenyl}-1,2,4-oxadiazol-3-yl)-1-ethyl-1H-indol-4-yl]propanoic acid

3-[7-(5-{5-Chloro-6-[(1-methylethyl)oxy]-3-pyridinyl}-1,2,4-oxadiazol-3-yl)-1H-indol-4-yl]propanoic acid

3-[7-(5-{5-Chloro-6-[(1-methylethyl)oxy]-3-pyridinyl}-1,2,4-oxadiazol-3-yl)-1-methyl-1H-indol-4-yl]propanoic acid

3-[7-(5-{3-Cyano-4-[(1-methylethyl)oxy]phenyl}-1,2,4-oxadiazol-3-yl)-1H-indol-4-yl]propanoic acid

3-[7-(5-{3-Cyano-4-[(1-methylethyl)oxy]phenyl}-1,2,4-oxadiazol-3-yl)-1-methyl-1H-indol-4-yl]propanoic acid

4-[7-(5-{3-Chloro-4-[(1-methylethyl)oxy]phenyl}-1,2,4-oxadiazol-3-yl)-1H-indol-4-yl]butanoic acid

4-[7-(5-{3-Chloro-4-[(1-methylethyl)oxy]phenyl}-1,2,4-oxadiazol-3-yl)-1-methyl-1H-indol-4-yl]butanoic acid

4-[7-(5-{5-Chloro-6-[(1-methylethyl)oxy]-3-pyridinyl}-1,2,4-oxadiazol-3-yl)-1H-indol-4-yl]butanoic acid

4-[7-(5-{5-Chloro-6-[(1-methylethyl)oxy]-3-pyridinyl}-1,2,4-oxadiazol-3-yl)-1-methyl-1H-indol-4-yl]butanoic acid

3-[4-(5-{3-Chloro-4-[(1-methylethyl)oxy]phenyl}-1,2,4-oxadiazol-3-yl)-1H-indol-7-yl]propanoic acid

3-[4-(5-{3-Chloro-4-[(1-methylethyl)oxy]phenyl}-1,2,4-oxadiazol-3-yl)-1-methyl-1H-indol-7-yl]propanoic acid

3-[4-(5-{3-Chloro-4-[(1-methylethyl)oxy]phenyl}-1,2,4-oxadiazol-3-yl)-1-ethyl-1H-indol-7-yl]propanoic acid

4-[4-(5-{3-Chloro-4-[(1-methylethyl)oxy]phenyl}-1,2,4-oxadiazol-3-yl)-1H-indol-7-yl]butanoic acid

4-[4-(5-{3-Chloro-4-[(1-methylethyl)oxy]phenyl}-1,2,4-oxadiazol-3-yl)-1-methyl-1H-indol-7-yl]butanoic acid

4-[4-(5-{3-Chloro-4-[(1-methylethyl)oxy]phenyl}-1,2,4-oxadiazol-3-yl)-1-ethyl-1H-indol-7-yl]butanoic acid

3-[4-(5-{5-Chloro-6-[(1-methylethyl)oxy]-3-pyridinyl}-1,2,4-oxadiazol-3-yl)-1H-indol-7-yl]propanoic acid

3-[4-(5-{5-Chloro-6-[(1-methylethyl)oxy]-3-pyridinyl}-1,2,4-oxadiazol-3-yl)-1-methyl-1H-indol-7-yl]propanoic acid

3-[4-(5-{5-Chloro-6-[(1-methylethyl)oxy]-3-pyridinyl}-1,2,4-oxadiazol-3-yl)-1-ethyl-1H-indol-7-yl]propanoic acid

4-[4-(5-{5-Chloro-6-[(1-methylethyl)oxy]-3-pyridinyl}-1,2,4-oxadiazol-3-yl)-1H-indol-7-yl]butanoic acid

4-[4-(5-{5-Chloro-6-[(1-methylethyl)oxy]-3-pyridinyl}-1,2,4-oxadiazol-3-yl)-1-methyl-1H-indol-7-yl]butanoic acid

4-[4-(5-{5-Chloro-6-[(1-methylethyl)oxy]-3-pyridinyl}-1,2,4-oxadiazol-3-yl)-1-ethyl-1H-indol-7-yl]butanoic acid

3-[4-(5-{3-Cyano-4-[(1-methylethyl)oxy]phenyl}-1,2,4-oxadiazol-3-yl)-1H-indol-7-yl]propanoic acid

3-[4-(5-{3-Cyano-4-[(1-methylethyl)oxy]phenyl}-1,2,4-oxadiazol-3-yl)-1-methyl-1H-indol-7-yl]propanoic acid

{[7-(5-{3-Chloro-4-[(1-methylethyl)oxy]phenyl}-1,2,4-oxadiazol-3-yl)-1-benzofuran-4-yl]oxy}acetic acid

4-{[7-(5-{3-Chloro-4-[(1-methylethyl)oxy]phenyl}-1,2,4-oxadiazol-3-yl)-1-benzofuran-4-yl]oxy}butanoic acid

{[4-(5-{3-Chloro-4-[(1-methylethyl)oxy]phenyl}-1,2,4-oxadiazol-3-yl)-1-benzofuran-7-yl]oxy}acetic acid

4-{[4-(5-{3-Chloro-4-[(1-methylethyl)oxy]phenyl}-1,2,4-oxadiazol-3-yl)-1-benzofuran-7-yl]oxy}butanoic acid

{[4-(5-{5-Chloro-6-[(1-methylethyl)oxy]-3-pyridinyl}-1,2,4-oxadiazol-3-yl)-1-benzofuran-7-yl]oxy}acetic acid

4-{[4-(5-{5-Chloro-6-[(1-methylethyl)oxy]-3-pyridinyl}-1,2,4-oxadiazol-3-yl)-1-benzofuran-7-yl]oxy}butanoic acid

3-[4-(5-{3-Chloro-4-[(1-methylethyl)oxy]phenyl}-1,2,4-oxadiazol-3-yl)-1-benzofuran-7-yl]propanoic acid

3-[4-(5-{5-Chloro-6-[(1-methylethyl)oxy]-3-pyridinyl}-1,2,4-oxadiazol-3-yl)-1-benzofuran-7-yl]propanoic acid

3-[7-(5-{3-Chloro-4-[(1-methylethyl)oxy]phenyl}-1,2,4-oxadiazol-3-yl)-1H-pyrrolo[2,3-c]pyridin-4-yl]propanoic acid

3-[7-(5-{3-Cyano-4-[(1-methylethyl)oxy]phenyl}-1,2,4-oxadiazol-3-yl)-1H-pyrrolo[2,3-c]pyridin-4-yl]propanoic acid or salts thereof.

Pharmaceutically acceptable derivatives of compounds of formula (I) include any pharmaceutically acceptable salt, ester or salt of such ester of a compound of formula (I) which, upon administration to the recipient is capable of providing (directly or indirectly) a compound of formula (I) or an active metabolic or residue thereof.

The compounds of formula (I) can form salts. It will be appreciated that for use in medicine the salts of the compounds of formula (I) should be pharmaceutically acceptable. Suitable pharmaceutically acceptable salts will be apparent to those skilled in the art and include those described in J. Pharm. Sci., 1977, 66, 1-19, such as acid addition salts formed with inorganic acids e.g. hydrochloric, hydrobromic, sulfuric, nitric or phosphoric acid; and organic acids e.g. succinic, maleic, acetic, fumaric, citric, tartaric, benzoic, p-toluenesulfonic, methanesulfonic or naphthalenesulfonic acid. Certain of the compounds of formula (I) may form acid addition salts with one or more equivalents of the acid. The present invention includes within its scope all possible stoichiometric and non-stoichiometric forms. Salts may also be prepared from pharmaceutically acceptable bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Salts derived from pharmaceutically acceptable organic bases include salts of primary, secondary, and tertiary amines; substituted amines including naturally occurring substituted amines; and cyclic amines. Particular pharmaceutically acceptable organic bases include arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, procaine, purines, theobromine, triethylamine, trimethylamine, tripropylamine, tris(hydroxymethyl)aminomethane (TRIS, trometamol) and the like. Salts may also be formed from basic ion exchange resins, for example polyamine resins. When the compound of the present invention is basic, salts may be prepared from pharmaceutically acceptable acids, including inorganic and organic acids. Such acids include acetic, benzenesulfonic, benzoic, camphorsulfonic, citric, ethanesulfonic, ethanedisulfonic, fumaric, gluconic, glutamic, hydrobromic, hydrochloric, isethionic, lactic, maleic, malic, mandelic, methanesulfonic, mucic, pamoic, pantothenic, phosphoric, propionic, succinic, sulfuric, tartaric, p-toluenesulfonic acid, and the like.

The compounds of formula (I) may be prepared in crystalline or non-crystalline form, and, if crystalline, may optionally be hydrated or solvated. This invention includes within its scope stoichiometric hydrates or solvates as well as compounds containing variable amounts of water and/or solvent.

Included within the scope of the invention are all salts, solvates, hydrates, complexes, polymorphs, prodrugs, radiolabelled derivatives, stereoisomers and optical isomers of the compounds of formula (I).

Pharmaceutically acceptable salts may be prepared conventionally by reaction with the appropriate acid or acid derivative.

The potencies and efficacies of the compounds of this invention for the S1P1 receptor can be determined by GTPγS assay or S1P1 Tango assay performed on the human cloned receptor as described herein. Compounds of formula (I) have demonstrated agonist activity at the S1P1 receptor, using functional assays described herein.

Compounds of formula (I) and their pharmaceutically acceptable salts are therefore of use in the treatment of conditions or disorders which are mediated via the S1P1 receptor. In particular the compounds of formula (I) and their pharmaceutically acceptable salts are of use in the treatment of multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes.

Compounds of formula (I) and their pharmaceutically acceptable salts are therefore of use in the treatment of multiple sclerosis.

Compounds of formula (I) and their pharmaceutically acceptable salts may also be of use in the treatment of Parkinson's Disease, Alzheimer's disease, Huntington's chorea, amyotrophic lateral sclerosis, spinal muscular atrophy, polyglutamine expansion disorders, vascular dementia, Down's syndrome, HIV dementia, dementia, ocular diseases including glaucoma, aged related macular degeneration, cataracts, traumatic eye injury, diabetic retinopathy, traumatic brain injury, stroke, tauopathies and hearing loss.

It is to be understood that “treatment” as used herein includes prophylaxis as well as alleviation of established symptoms.

Thus the invention also provides a compound of formula (I) or a pharmaceutically acceptable salt thereof, for use as a therapeutic substance, in particular in the treatment of the conditions or disorders mediated via the S1P1 receptor. In particular the invention provides a compound of formula (I) or a pharmaceutically acceptable salt thereof for use as a therapeutic substance in the treatment of multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes. The invention further provides a method of treatment of conditions or disorders in mammals including humans which can be mediated via the S1P1 receptor, which comprises administering to the sufferer a therapeutically safe and effective amount of a compound of formula (I) or a pharmaceutically acceptable salt thereof.

Compounds of formula (I) and their pharmaceutically acceptable salts are of use as therapeutic substances in the treatment of multiple sclerosis.

In another aspect, the invention provides for the use of a compound of formula (I) or a pharmaceutically acceptable salt thereof in the manufacture of a medicament for use in the treatment of the conditions or disorders mediated via the S1P1 receptor

The invention provides a method of treatment of multiple sclerosis, which comprises administering to the sufferer a therapeutically safe and effective amount of a compound of formula (I) or a pharmaceutically acceptable salt thereof.

In order to use the compounds of formula (I) and pharmaceutically acceptable salts thereof in therapy, they will normally be formulated into a pharmaceutical composition in accordance with standard pharmaceutical practice. The present invention also provides a pharmaceutical composition, which comprises a compound of formula (I) or a pharmaceutically acceptable salt thereof, and a pharmaceutically acceptable carrier or excipient.

In a further aspect, the present invention provides a process for preparing a pharmaceutical composition, the process comprising mixing a compound of formula (I) or a pharmaceutically acceptable salt thereof and a pharmaceutically acceptable carrier or excipient.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusible solutions or suspensions or suppositories. Orally administrable compositions are generally preferred.

Tablets and capsules for oral administration may be in unit dose form, and may contain conventional excipients, such as binding agents (e.g. pregelatinised maize starch, polyvinylpyrrolidone or hydroxypropyl methylcellulose); fillers (e.g. lactose, microcrystalline cellulose or calcium hydrogen phosphate); tabletting lubricants (e.g. magnesium stearate, talc or silica); disintegrants (e.g. potato starch or sodium starch glycollate); and acceptable wetting agents (e.g. sodium lauryl sulphate). The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents (e.g. sorbitol syrup, cellulose derivatives or hydrogenated edible fats), emulsifying agents (e.g. lecithin or acacia), non-aqueous vehicles (which may include edible oils e.g. almond oil, oily esters, ethyl alcohol or fractionated vegetable oils), preservatives (e.g. methyl or propyl-p-hydroxybenzoates or sorbic acid), and, if desired, conventional flavourings or colorants, buffer salts and sweetening agents as appropriate. Preparations for oral administration may be suitably formulated to give controlled release of the active compound.

For parenteral administration, fluid unit dosage forms are prepared utilising a compound of the invention or pharmaceutically acceptable salts thereof and a sterile vehicle. Formulations for injection may be presented in unit dosage form e.g. in ampoules or in multi-dose, utilising a compound of the invention or pharmaceutically acceptable derivatives thereof and a sterile vehicle, optionally with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilisation cannot be accomplished by filtration. The compound can be sterilised by exposure to ethylene oxide before suspension in a sterile vehicle. Advantageously, a surfactant or wetting agent is included in the composition to facilitate uniform distribution of the compound.

Lotions may be formulated with an aqueous or oily base and will in general also contain one or more emulsifying agents, stabilising agents, dispersing agents, suspending agents, thickening agents, or colouring agents. Drops may be formulated with an aqueous or non-aqueous base also comprising one or more dispersing agents, stabilising agents, solubilising agents or suspending agents. They may also contain a preservative.

The compounds of formula (I) or pharmaceutically acceptable salts thereof may also be formulated in rectal compositions such as suppositories or retention enemas, e.g. containing conventional suppository bases such as cocoa butter or other glycerides.

The compounds of formula (I) or pharmaceutically acceptable salts thereof may also be formulated as depot preparations. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

For intranasal administration, the compounds of formula (I) or pharmaceutically acceptable salts thereof, may be formulated as solutions for administration via a suitable metered or unitary dose device or alternatively as a powder mix with a suitable carrier for administration using a suitable delivery device. Thus compounds of formula (I) or pharmaceutically acceptable salts thereof may be formulated for oral, buccal, parenteral, topical (including ophthalmic and nasal), depot or rectal administration or in a form suitable for administration by inhalation or insufflation (either through the mouth or nose).

The compounds of formula (I) or pharmaceutically acceptable salts thereof may be formulated for topical administration in the form of ointments, creams, gels, lotions, pessaries, aerosols or drops (e.g. eye, ear or nose drops). Ointments and creams may, for example, be formulated with an aqueous or oily base with the addition of suitable thickening and/or gelling agents. Ointments for administration to the eye may be manufactured in a sterile manner using sterilised components.

The composition may contain from 0.1% to 99% by weight, preferably from 10 to 60% by weight, of the active material, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, 1.0 to 500 mg or 1.0 to 200 mg and such unit doses may be administered more than once a day, for example two or three times a day.

Compounds of formula (I) or pharmaceutically acceptable salts thereof may be used in combination preparations. For example, the compounds of the invention may be used in combination with cyclosporin A, methotrexate, steriods, rapamycin, proinflammatory cytokine inhibitors, immunomodulators including biologicals or other therapeutically active compounds.

The subject invention also includes isotopically-labeled compounds, which are identical to those recited in formulas I and following, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine, iodine, and chlorine, such as ³H, ¹¹C, ¹⁴C, ¹⁸F, ¹²³I and ¹²⁵I.

Compounds of the present invention and pharmaceutically acceptable saltss of said compounds that contain the aforementioned isotopes and/or other isotopes of other atoms are within the scope of the present invention. Isotopically-labeled compounds of the present invention, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ⁸F isotopes are particularly useful in PET (positron emission tomography), and ¹²⁵I isotopes are particularly useful in SPECT (single photon emission computerized tomography), all useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formula (I) and following of this invention can generally be prepared by carrying out the procedures disclosed in the Examples below, by substituting a readily available isotopically labelled reagent for a non-isotopically labeled reagent.

All publications, including but not limited to patents and patent applications, cited in this specification are herein incorporated by reference as if each individual publication were specifically and individually indicated to be incorporated by reference herein as though fully set forth.

The following Descriptions and Examples illustrate the preparation of compounds of the invention.

Abbreviations

g—grams

mg—milligrams

ml—millilitres

min—minute

ul—microlitres

MeCN—acetonitrile

MeOH—methanol

EtOH—ethanol

Et₂O—diethyl ether

EtOAc—ethyl acetate

DABCO—1,4-diazabiclo[2,2,2]octane

DCM—dichloromethane

DIAD—diisopropyl azodicarboxylate

DME—1,2-bis(methyloxy)ethane

DMF—N,N-dimethylformamide

DMSO—dimethylsulphoxide

EDAC—N-(3-Dimethylaminopropyl)-N′-ethylcarbodiimide hydrochloride

EDC—N-(3-Dimethylaminopropyl)-N′-ethylcarbodiimide hydrochloride

EDCI—N-(3-Dimethylaminopropyl)-N′-ethylcarbodiimide hydrochloride

HOBT/HOBt—Hydroxybenzotriazole

IPA—isopropylalcohol

NCS—N-chlorosuccinimide

PyBOP—Benzotriazol-1-yl-oxytripyrrolidinophosphonium hexafluorophosphate

THF—tetrahydrofuran

dba—dibenzylidene acetone

RT—room temperature

° C.—degrees Celsius

M—Molar

H—proton

s—singlet

d—doublet

t—triplet

q—quartet

MHz—megahertz

MeOD—deuterated methanol

LCMS—Liquid Chromatography Mass Spectrometry

LC/MS—Liquid Chromatography Mass Spectrometry

MS—mass spectrometry

ES—Electrospray

MH⁺—mass ion+H⁺

MDAP—mass directed automated preparative liquid chromatography.

sat.—saturated

Chromatography

Unless stated otherwise, all chromatography was carried out using silica columns.

General Chemistry Section

The intermediates for the preparation of the examples may not necessarily have been prepared from the specific batch of precursor described.

Description for D1

### 4,7-Dibromo-1H-indole (D1)

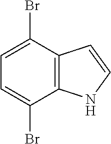

Under nitrogen atmosphere, −40° C.˜50° C. The solution of 1,4-dibromo-2-nitrobenzene (14.0 g) in THF (30 mL) was added dropwise to the bromo(ethenyl)magnesium (26.2 g) (200 mL, 1.0 M in THF) in 20 min. The resulting mixture was stirred at −40° C.˜50° C. for 2.5 hours, and the reaction was quenched with saturated ammonium chloride. The mixture was extracted with ethyl acetate and the oganic layer was washed with brine, dried over sodium sulphate. The crude product was purified by silica gel column on ISCO eluting with 10% EtOAc/Petroleum to obtain 8.0 g 4, 7-dibromo-1H-indole (D1).

MS (ES): C₈H₅Br₂N requires 273.0 (⁷⁹Br), 275.0 (⁸¹Br); found 275.8 (M+H⁺, ⁷⁹Br), 277.1 (M+H⁺, ⁸¹Br).

Description for D2A and D2B

### 4-Bromo-1H-indole-7-carbonitrile (D2A)

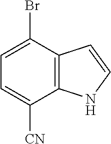

### 7-Bromo-1H-indole-4-carbonitrile (D2B)

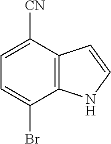

To a solution of 4, 7-dibromo-1H-indole (1.5 g) in DMF (10 mL) in a microwave vial, CuCN (0.49 g) was added. The vial was heated at 170° C. on the microwave for 1 h. Then the solvent was concentrated in vacuum to give a black residue, then puirified by flash column chromatography 5% EtOAc/Petroleum to obtain 90 mg 4-bromo-1H-indole-7-carbonitrile (D2A) and 40 mg 7-bromo-1H-indole-4-carbonitrile (D2B).

MS (ES): C₉H₅BrN₂ requires 220.0 (⁷⁹Br), 222.0 (⁸¹Br); found 221.0 (M+H⁺, ⁷⁹Br), 223.1 (M+H⁺, ⁸¹Br).

Description for D3

### 4-Bromo-1-[(4-methylphenyl)sulfonyl]-1H-indole-7-carbonitrile (D3)

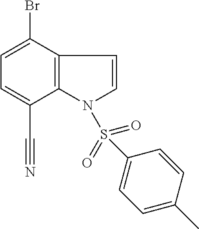

A mixture of 4-bromo-1H-indole-7-carbonitrile (1.0 g), Et₃N (7.6 mL), and DMAP (55 mg) in DCM (10 mL) was stirred at 0° C., 4-methylbenzenesulfonyl chloride (2.59 g) was added in one port. After addition completed, the mixture was stirred at 25° C. overnight. Aqueous NaHCO₃ solution (20 mL) and DCM (20 mL) were added; the DCM fraction was separated and dried over anhydrous magnesium sulphate. The dried solution was concentrated in vacuum and the resulting residue was purified by Teledyne ISCO (15% EtOAc in Petroleum) to afford 4-bromo-1-[(4-methylphenyl)sulfonyl]-1H-indole-7-carbonitrile (D3) (1.05 g) as a grey solid.

MS (ES): C₁₆H₁₁BrN₂O₂S requires 374.1 (⁷⁹Br), 376.0 (⁸¹Br); found 375.0 (M+H⁺, ⁷⁹Br), 377.1 (M+H⁺, ⁸¹Br).

Description for D4

### Ethyl 3-{7-cyano-1-[(4-methylphenyl)sulfonyl]-1H-indol-4-yl}propanoate (D4)

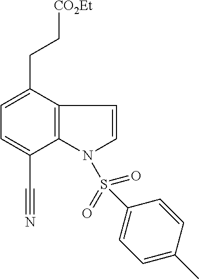

A mixture of 4-bromo-1-[(4-methylphenyl)sulfonyl]-1H-indole-7-carbonitrile (400 mg), bromo[3-(ethyloxy)-3-oxopropyl]zinc (525 mg), (Ph₃P)₄Pd (61 mg) in THF (10 mL) was stirred under nitrogen atmoshpere at 85° C. overnight. Aqueous NaOH solution (15 mL) was added to the mixture after it was cooled to RT. Stirring was continued for 15 min. The inorganics was filtered off and the resulting filtrate was extracted with EtOAc (2×10 mL). The organic fractions were combined. The combined solution was dried over anhydrous magnesium sulfate. The dried solution was concentrated in vacuum and the resulting residue was purified by Teledyne ISCO (15% EtOAc in Petroleum) to afford ethyl 3-{7-cyano-1-[(4-methylphenyl)sulfonyl]-1H-indol-4-yl}propanoate (D4) (180 mg) as a white solid.

MS (ES): C₂₁H₂₀N₂O₄S requires 396.1; found 397.1 (M+H⁺).

Description for D5

### Ethyl 4-{7-cyano-1-[(4-methylphenyl)sulfonyl]-1H-indol-4-yl}butanoate (D5)

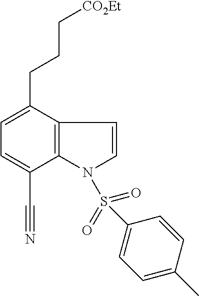

A mixture of 4-bromo-1-[(4-methylphenyl)sulfonyl)-1H-indole-7-carbonitrile (1.0 g), bromo[4-(ethyloxy)-4-oxobutyl]zinc (1.0 g), Pd(Ph₃P)₄ (0.3 g) in THF (10 mL) was stirred under nitrogen atmoshpere at 85° C. overnight. Aqueous NaOH solution (15 mL) was added to the mixture after it was cooled to RT. Stirring was continued for 15 min. The inorganics was filtered off and the resulting filtrate was extracted with EtOAc (2×10 mL). The organic fractions were combined. The combined solution was dried over anhydrous magnesium sulfate. The dried solution was concentrated in vacuum and the resulting residue was purified by Teledyne ISCO (15% EtOAc in Petroleum) to afford ethyl 4-{7-cyano-1-[(4-methylphenyl)sulfonyl]-1H-indol-4-yl}butanoate (D5) (680 mg) as a white solid.

MS (ES): C₂₂H₂₂N₂O₄S requires 410; found 411.1 (M+H⁺).

Description for D6

### Ethyl 3-{7-[(hydroxyamino)(imino)methyl]-1-[(4-methylphenyl)sulfonyl]-1H-indol-4-yl}propanoate (D6)

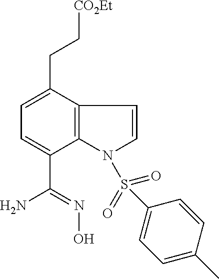

The mixture of hydroxylamine hydrochloride (95 mg), ethyl 3-{7-cyano-1-[(4-methylphenyl)sulfonyl]-1H-indol-4-yl}propanoate (180 mg), and sodium carbonate (250 mg) in Ethanol (2 mL) was sealed and heated in Biotage Initiator using initial normal to 120° C. for 60 min. EtOAc (10 mL) was added and the resulting mixture was washed with water (10 mL). The organic fraction was dried over anhydrous magnesium sulfate. The dried solution was concentrated in vacuum to afford ethyl 3-{7-[(hydroxyamino)(imino)methyl]-1-[(4-methylphenyl)sulfonyl]-1H-indol-4-yl}propanoate (D6) (56 mg).

MS (ES): C₂₁H₂₃N₃O₅S requires 429; found 430.1 (M+H⁺).

Description for D7

### Ethyl 4-{7-[(hydroxyamino)(imino)methyl]-1-[(4-methylphenyl)sulfonyl]-1H-indol-4-yl}butanoate (D7)

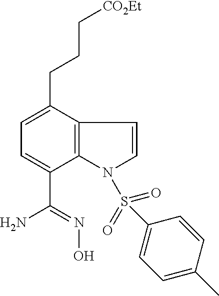

The mixture of ethyl 4-{7-cyano-1-[(4-methylphenyl)sulfonyl] -1H-indol-4-yl}butanoate (760 mg), hydroxylamine hydrochloride (193 mg) and sodium carbonate (467 mg) in ethanol (15 mL) was stirred at 85° C. for 6 hours. The mixture was cooled to RT and filtered. The filtrate was concentrated in vacuum to afford ethyl 4-{7-[(hydroxyamino)(imino)methyl]-1-[(4-methylphenyl)sulfonyl]-1H-indol-4-yl}butanoate (D7) (760 mg).

MS (ES): C₂₂H₂₅N₃O₅S requires 443; found 444.2 (M+H⁺).

Description for D8

### Ethyl 3-[7-(5-{3-chloro-4-[(1-methylethyl)oxy]phenyl}-1,2,4-oxadiazol-3-yl)-1H-indol-4-yl]propanoate (D8)

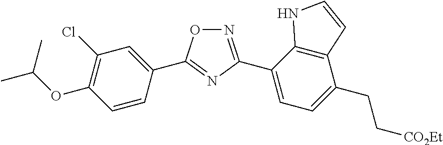

Under nitrogen atmosphere, the mixture of ethyl 3-{7-[(E)-(hydroxyamino)(imino)methyl]-1-[(4-methylphenyl)sulfonyl]-1H-indol-4-yl}propanoate (141 mg), 3-chloro-4-[(1-methylethyl)oxy]benzoic acid (141 mg), EDCI (189 mg), triethylamine (0.183 mL) and 4-dimethylaminopyridine (40 mg) in THF (10 mL) was stirred at rt for 48 hours. The mixture was evaporated to dryness and was dissolved in ethyl acetate, washed with water and brine, dried over sodium sulphate, evaporated to dryness. The residue yellow oil 205 mg ethyl 3-{7-[(Z)-{[({3-chloro-4-[(1-methylethyl)oxy]phenyl}carbonyl)oxy]amino)(imino)methyl]-1-[(4-methylphenyl)sulfonyl]-1H-indol-4-yl}propanoate was obtained. TBAF (86 mg) was added to the solution of ethyl 3-{7-[(Z)-{[({3-chloro-4-[(1-methylethyl)oxy]phenyl}carbonyl)oxy]amino}(imino)methyl]-1-[(4-methylphenyl)sulfonyl]-1H-indol-4-yl}propanoate (205 mg) in THF. The reaction mixture was heated up reflux 3 hours. After removing the solvent, the residue was partitioned between ethyl acetate and water. The organic layer was washed with brine, drie over sodium sulphate and evaporated under reduced pressure. The crude product was purified by column chromatgraphy on silica gel eluting with EtOAc: Petroleum=1:10 obtaining 58 mg ethyl 3-[7-(5-{3-chloro-4-[(1-methylethyl)oxy]phenyl}-1,2,4-oxadiazol-3-yl)-1H-indol-4-yl]propanoate (D8).

MS (ES): C₂₄H₂₄CIN₃O₄ requires 453.1; found 454.1 (M+H⁺).

Description for D9

### Ethyl 3-(7-(5-{5-chloro-6-[(1-methylethyl)oxy]-3-pyridinyl}-1,2,4-oxadiazol-3-yl)-1H-indol-4-yl]propanoate (D9)

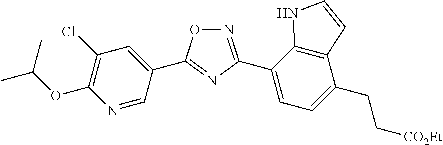

The mixture of Ethyl 3-{7-[(hydroxyamino)(imino)methyl]-1-[(4-methylphenyl)sulfonyl]-1H-indol-4-yl}propanoate (80 mg), 5-chloro-6-[(1-methylethyl)oxy]-3-pyridinecarboxylic acid (40 mg), EDC (71 mg), HOBT (6 mg), Et₃N (0.065 mL) in THF (5 mL) was stirred at RT overnight. EtOAc (10 mL) was added and the organic solution was washed with water (10 mL). The organic fraction was dried over anhydrous magnesium sulfate. The dried solution was concentrated in vacuum and the resulting residue was purified by Teledyne ISCO (15% EtOAc in Petroleum) to afford ethyl 3-{7-[(hydroxyamino)(imino)methyl]-1-[(4-methylphenyl)sulfonyl]-1H-indol-4-yl}propanoate (110 mg) as white solid.

MS (ES): C₃₀H₃₁CIN₄O₇S requires 626; found 627.2 (M+H⁺).

The mixture of ethyl 3-{7-[(hydroxyamino)(imino)methyl]-1-[(4-methylphenyl)sulfonyl]-1H-indol-4-yl}propanoate (110 mg) and TBAF (229 mg) in THF (10 mL) was stirred at 85° C. for 5 hours. EtOAc (10 mL) was added and the resulting solution was washed with water (2×3 mL). The organic fractions were combined. The combined solution was dried over anhydrous magnesium sulfate. The dried solution was concentrated. The resulting residue was purified by Teledyne ISCO (10% EtOAc in Petroleum) to afford ethyl 3-[7-(5-{5-chloro-6-[(1-methylethyl)oxy]-3-pyridinyl}-1,2,4-oxadiazol-3-yl)-1H-indol-4-yl]propanoate (D9) (30 mg) as a yellow oil.

MS (ES): C₂₃H₂₃CIN₄O₄ requires 454; found 455.1 (M+H⁺).

Description for D10

### Ethyl 3-[7-(5-{5-chloro-6-[(1-methylethyl)oxy]-3-pyridinyl}-1,2,4-oxadiazol-3-yl)-1-methyl-1H-indol-4-yl]propanoate (D10)

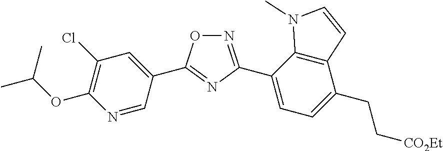

The mixture of ethyl ethyl 3-[7-(5-{5-chloro-6-[(1-methylethyl)oxy]-3-pyridinyl}-1,2,4-oxadiazol-3-yl)-1H-indol-4-yl]propanoate (210 mg), dimethyl carbonate (832 mg) and DABCO (104 mg) in DMF (2 mL) was stirred at 120° C. under nitrogen atmosphere for 5 hours. EtOAc (20 mL) was added and the organic solution was washed with water (2×5 mL). The organic fraction was dried over anhydrous magnesium sulfate. The dried solution was concentrated in vacuum and the resulting residue was purified by Teledyne ISCO (30% EtOAc in Petroleum) to afford ethyl 3-[7-(5-{5-chloro-6-[(1-methylethyl)oxy]-3-pyridinyl}-1,2,4-oxadiazol-3-yl)-1-methyl-1H-indol-4-yl]propanoate (D10) (100 mg) as a yellow oil.

MS (ES): C₂₄H₂₅CIN₄O₄ requires 468; found 469.2 (M+H⁺).

Description for D11

### Ethyl 3-[7-(5-{3-cyano-4-[(1-methylethyl)oxy]phenyl}-1,2,4-oxadiazol-3-yl)-1H-indol-4-yl]propanoate (D11)

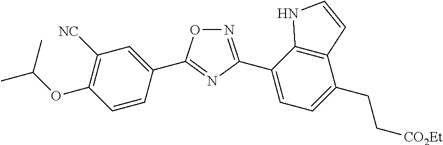

The mixture of ethyl 3-{7-[(Z)-(hydroxyamino)(imino)methyl]-1-[(4-methylphenyl)sulfonyl]-1H-indol-4-yl}propanoate (790 mg),3-cyano-4-[(1-methylethyl)oxy]benzoic acid (377 mg), Et₃N (0.641 mL), EDC (705 mg), HOBT (56.3 mg) in THF (10 mL) was stirred at RT for 2 hours. EtOAc (60 mL) was added and the organic solution was washed with water (20 mL). The organic fraction was dried over anhydrous magnesium sulfate. The dried solution was concentrated in vacuum to afford ethyl 3-{7-[(Z)-{[({3-cyano-4-[(1-methylethyl)oxy]phenyl}carbonyl)oxy]amino}(imino)methyl]-1-[(4-methylphenyl)sulfonyl]-1H-indol-4-yl}propanoate (1.1 g) as a yellow oil. MS (ES): C₃₂H₃₂N₄O₇S requires 616; found 617.2 (M+H⁺).

The mixture of ethyl 3-{7-[(Z)-{[({3-cyano-4-[(1-methylethyl)oxy]phenyl}carbonyl)oxy]amino)(imino)methyl}-1-[(4-methylphenyl)sulfonyl]-1H-indol-4-yl}propanoate (1134 mg), TBAF (1923 mg) in THF (10 mL) was stirred at 85° C. for 2 hours. EtOAc (60 mL) was added and the resulting solution was washed with water (2×20 mL). The organic fractions were combined. The combined solution was dried over anhydrous magnesium sulfate. The dried solution was concentrated. The resulting residue was purified by Teledyne ISCO (25% EtOAc in Petroleum) to afford ethyl 3-[7-(5-{3-cyano-4-[(1-methylethyl)oxy]phenyl}-1,2,4-oxadiazol-3-yl)-1H-indol-4-yl]propanoate (D11) (510 mg) as a yellow oil.

MS (ES): C₂₅H₂₄N₄O₄ requires 444; found 445.2 (M+H⁺).

Description for D12

### Ethyl 3-(7-cyano-1H-indol-4-yl)propanoate (D12)

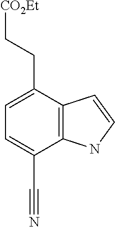

The mixture of ethyl 3-{7-cyano-1-[(4-methylphenyl)sulfonyl]-1H-indol-4-yl}propanoate (850 mg) and TBAF (1682 mg) in THF (10 mL) was stirred at 85° C. for 3 hours. EtOAc (60 mL) was added and the resulting solution was washed with water (3×20 mL). The organic fractions were combined. The combined solution was dried over anhydrous magnesium sulfate. The dried solution was concentrated under vacuum to afford ethyl 3-(7-cyano-1H-indol-4-yl)propanoate (D12) (520 mg) as a yellow oil. MS (ES): C₁₄H₁₄N₂O₂ requires 242; found 243.4 (M+H⁺).

Description for D13

### Ethyl 3-(7-cyano-1-methyl-1H-indol-4-yl)propanoate (D13)

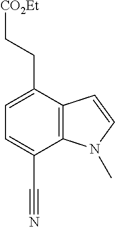

The mixture of ethyl 3-(7-cyano-1H-indol-4-yl)propanoate (519 mg), KOH (361 mg), Me₂SO₄ (0.512 mL) in acetonitrile (5 mL) was stirred at 80° C. for 2 hours. EtOAc (40 mL) was added and the resulting solution was washed with water (3×10 mL). The organic fractions were combined. The combined solution was dried over anhydrous magnesium sulfate. The dried solution was concentrated. The resulting residue was purified by Teledyne ISCO (15% EtOAc in Petroleum) to afford ethyl 3-(7-cyano-1-methyl-1H-indol-4-yl)propanoate (D13) (420 mg) as a white oil.

MS (ES): C₁₅H₁₆N₂O₂ requires 256; found 257.2 (M+H⁺).

Description for D14

### Ethyl 3-{7-[(E)-(hydroxyamino)(imino)methyl]-1-methyl-1H-indol-4-yl}propanoate (D14)

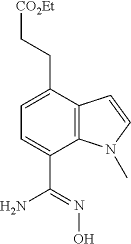

The mixture of ethyl 3-(7-cyano-1-methyl-1H-indol-4-yl)propanoate (420 mg), hydroxylamine hydrochloride (342 mg), sodium bicarbonate (413 mg) in ethanol (10 mL) was stirred at 80° C. overnight. The inorganics was removed by filtration. The filtrate was concentrated under vacuum and the resulting residue was purified by Teledyne ISCO (35% EtOAc in Petroleum) to afford ethyl 3-{7-[(E)-(hydroxyamino)(imino)methyl]-1-methyl-1H-indol-4-yl}propanoate (D14) (230 mg) as a white oil.

MS (ES): C₁₅H₁₉N₃O₃ requires 289; found 290.2 (M+H⁺).

Description for D15

### Ethyl 3-[7-(5-{3-cyano-4-[(1-methylethyl)oxy]phenyl}-1,2,4-oxadiazol-3-yl)-1-methyl-1H-indol-4-yl]propanoate (D15)

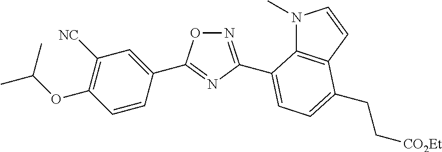

The mixture of ethyl 3-{7-[(Z)-(hydroxyamino)(imino)methyl]-1-methyl-1H-indol-4-yl}propanoate (230 mg), 3-cyano-4-[(1-methylethyl)oxy]benzoic acid (163 mg), EDC (305 mg), HOBT (24 mg), Et₃N (0.277 mL) in THF (10 mL) was stirred at RT overnight. EtOAc (60 mL) was added and the organic solution was washed with water (20 mL). The organic layer was dried over anhydrous magnesium sulfate. The dried solution was concentrated in vacuum to afford ethyl 3-{7-[(E)-{[({3-cyano-4-[(1-methylethyl)oxy]phenyl}carbonyl)oxy]amino}(imino)methyl]-1-methyl-1H-indol-4-yl}propanoate (380 mg) as a yellow oil. MS (ES): C₂₆H₂₈N₄O₅ requires 476; found 477.2 (M+H⁺).

The mixture of ethyl 3-{7-[(E)-{[({3-cyano-4-[(1-methylethyl)oxy]phenyl}carbonyl)oxy]amino}(imino)methyl]-1-methyl-1H-indol-4-yl}propanoate (379 mg) and TBAF (832 mg) in THF (10 mL) was stirred at 85° C. for 2 hours. EtOAc (60 mL) was added and the resulting solution was washed with water (2×15 mL). The organic fractions were combined. The combined solution was dried over anhydrous magnesium sulfate. The dried solution was concentrated. The resulting residue was purified by Teledyne ISCO (30% EtOAc in Petroleum) to afford ethyl 3-[7-(5-{3-cyano-4-[(1-methylethyl)oxy]phenyl}-1,2,4-oxadiazol-3-yl)-1-methyl-1H-indol-4-yl]propanoate (D15) (150 mg) as a white solid.

MS (ES): C₂₆H₂₆N₄O₄ requires 458; found 459.2 (M+H⁺).

Description for D16

### Ethyl 4-{7-(5-{3-chloro-4-[(1-methylethyl)oxy]phenyl}-1,2,4-oxadiazol-3-yl)-14(4-methylphenyl)sulfonyl]-1H-indol-4-yl}butanoate (D16)

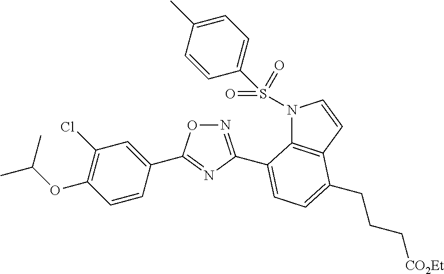

The mixture of 4-{7-[(hydroxyamino)(imino)methyl]-1-[(4-methylphenyl)sulfonyl]-1H-indol-4-yl}butanoate (400 mg), 5-chloro-6-[(1-methylethyl)oxy]-3-pyridinecarboxylic acid (106 mg), EDC (143 mg), HOBT (15 mg), Et₃N (125 mg) in THF (10 mL) was stirred at RT overnight. EtOAc (20 mL) was added and the organic solution was washed with water (10 mL). The organic fraction was dried over anhydrous magnesium sulfate. The dried solution was concentrated in vacuum and the resulting residue was purified by Teledyne ISCO (15% EtOAc in Petroleum) to afford ethyl 4-{7-[{[({3-chloro-4-[(1-methylethyl)oxy]phenyl}carbonyl)oxy]amino}(imino)methyl]-1-[(4-methylphenyl)sulfonyl]-1H-indol-4-yl}butanoate (400 mg) as white solid. MS (ES): C₃₂H₃₄CIN₃O₇S requires 639; found 640.2 (M+H⁺).

The solution of ethyl 4-{7-[{[({3-chloro-4-[(1-methylethyl)oxy]phenyl}carbonyl)oxy]amino}(imino)methyl]-1-[(4-methylphenyl)sulfonyl]-1H-indol-4-yl}butanoate (400 mg) in dioxane (5 mL) was stirred at 110° C. for 6 hours. EtOAc (10 mL) was added and the resulting solution was washed with water (2×5 mL). The organic fraction was dried over anhydrous magnesium sulfate. The dried solution was concentrated and the resulting residue was purified by Teledyne ISCO (20% EtOAc in Petroleum) to afford ethyl 4-{7-(5-{3-chloro-4-[(1-methylethyl)oxy]phenyl}-1,2,4-oxadiazol-3-yl)-1-[(4-methylphenyl)sulfonyl]-1H-indol-4-yl}butanoate (D16) (170 mg) as a yellow solid.

MS (ES): C₃₂H₃₂CIN₃O₆S requires 621; found 622.2 (M+H⁺).

Description for D17

### Ethyl 4-[7-(5-{3-chloro-4-[(1-methylethyl)oxy]phenyl}-1,2,4-oxadiazol-3-yl)-1H-indol-4-yl]butanoate (D17)

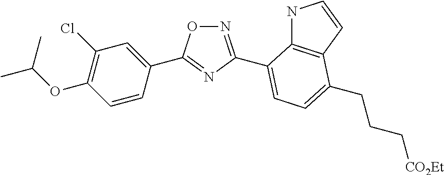

The mixture of ethyl 4-{7-(5-{3-chloro-4-[(1-methylethyl)oxy]phenyl}-1,2,4-oxadiazol-3-yl)-1-[(4-methylphenyl)sulfonyl]-1H-indol-4-yl}butanoate (80 mg) and TBAF (80 mg) in THF (5 mL) was stirred at 70° C. for 3 hours. EtOAc (10 mL) was added and the resulting solution was washed with water (2×3 mL). The organic fractions were combined. The combined solution was dried over anhydrous magnesium sulfate. The dried solution was concentrated. The resulting residue was purified by Teledyne ISCO (20% EtOAc in Petroleum) to afford ethyl 4-[7-(5-{3-chloro-4-[(1-methylethyl)oxy]phenyl}-1,2,4-oxadiazol-3-yl)-1H-indol-4-yl]butanoate (D17)(60 mg) as a yellow oil.

MS (ES): C₂₅H₂₆CIN₃O₄ requires 467; found 468.2 (M+H⁺).

Description for D18

### Ethyl 4-(7-(5-{3-chloro-4-[(1-methylethyl)oxy]phenyl}-1,2,4-oxadiazol-3-yl)-1-methyl-1H-indol-4-yl]butanoate (D18)

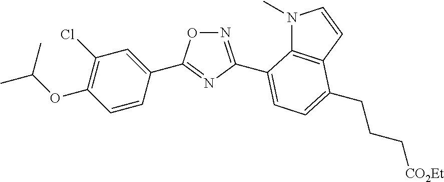

The mixture of ethyl ethyl 4-[7-(5-{3-chloro-4-[(1-methylethyl)oxy]phenyl}-1,2,4-oxadiazol-3-yl)-1H-indol-4-yl]butanoate (110 mg), dimethyl carbonate (423 mg) and DABCO (26 mg) in DMF (2 mL) was stirred at 120° C. under nitrogen atmosphere for 5 hours. EtOAc (20 mL) was added and the organic solution was washed with water (2×5 mL). The organic fraction was dried over anhydrous magnesium sulfate. The dried solution was concentrated in vacuum and the resulting residue was purified by Teledyne ISCO (25% EtOAc in Petroleum) to afford ethyl 4-[7-(5-{3-chloro-4-[(1-methylethyl)oxy]phenyl}-1,2,4-oxadiazol-3-yl)-1-methyl-1H-indol-4-yl]butanoate (D18) (113 mg) as a yellow oil.

MS (ES): C₂₆H₂₈CIN₃O₄ requires 481; found 482.2 (M+H⁺).

Description for D19

### Ethyl 4-[7-(5-{5-chloro-6-[(1-methylethyl)oxy]-3-pyridinyl}-1,2,4-oxadiazol-3-yl)-1H-indol-4-yl]butanoate (D19)

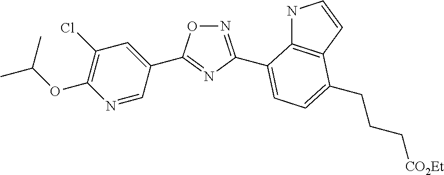

The mixture of ethyl 4-{7-[(Z)-(hydroxyamino)(imino)methyl]-1-[(4-methylphenyl)sulfonyl]-1H-indol-4-yl}butanoate (270 mg), 5-chloro-6-[(1-methylethyl)oxy]-3-pyridinecarboxylic acid (151 mg), EDC (268 mg), HOBT (21 mg), Et₃N (177 mg) in THF (10 mL) was stirred at RT overnight. EtOAc (20 mL) was added and the organic solution was washed with water (10 mL). The organic fraction was dried over anhydrous magnesium sulfate. The dried solution was concentrated in vacuum to afford crude product ethyl 4-{7-[(Z)-{[({5-chloro-6-[(1-methylethyl)oxy]-3-pyridinyl)carbonyl)oxy]amino}(imino)methyl]-1-[(4-methylphenyl)sulfonyl]-1H-indol-4-yl}butanoate (448 mg).

MS (ES): C₃₁H₃₃CIN₄O₇S requires 640; found 641.2 (M+H⁺), 663.2 (M+Na⁺)

The mixture of ethyl 4-[7-(5-{5-chloro-6-[(1-methylethyl)oxy]-3-pyridinyl}-1,2,4-oxadiazol-3-yl)-1H-indol-4-yl]butanoate (448 mg) and TBAF (913 mg) in THF (5 mL) was stirred at 85° C. for 2 hours. EtOAc (10 mL) was added and the resulting solution was washed with water (2×3 mL). The organic fractions were combined. The combined solution was dried over anhydrous magnesium sulfate. The dried solution was concentrated. The resulting residue was purified by Teledyne ISCO (25% EtOAc in Petroleum) to afford ethyl 4-[7-(5-{5-chloro-6-[(1-methylethyl)oxy]-3-pyridinyl}-1,2,4-oxadiazol-3-yl)-1H-indol-4-yl]butanoate (D19) (188 mg) as a yellow oil.

MS (ES): C₂₄H₂₅CIN₄O₄ requires 468; found 469.2 (M+H⁺).

Description for D20

### Ethyl 4-[7-(5-{5-chloro-6-[(1-methylethyl)oxy]-3-pyridinyl}-1,2,4-oxadiazol-3-yl)-1-methyl-1H-indol-4-yl]butanoate (D20)

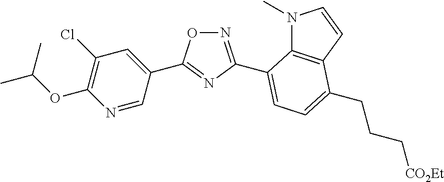

The mixture of ethyl ethyl 4-[7-(5-{5-chloro-6-[(1-methylethyl)oxy]-3-pyridinyl}-1,2,4-oxadiazol-3-yl)-1H-indol-4-yl]butanoate (120 mg), dimethyl carbonate (461 mg) and DABCO (28 mg) in DMF (2 mL) was stirred at 120° C. under nitrogen atmosphere for 5 hours. EtOAc (20 mL) was added and the organic solution was washed with water (2×5 mL). The organic fraction was dried over anhydrous magnesium sulfate. The dried solution was concentrated in vacuum and the resulting residue was purified by Teledyne ISCO (25% EtOAc in Petroleum) to afford ethyl 4-[7-(5-{5-chloro-6-[(1-methylenthyl)oxy]-3-pyridinyl}-1,2,4-oxadiazol-3-yl)-1-methyl-1H-indol-4-yl]butanoate (D20) (85 mg) as a yellow oil.

MS (ES): C₂₅H₂₇CIN₄O₄ requires 482; found 483.2 (M+H⁺).

Description for D21

### 7-Bromo-1-methyl-1H-indole-4-carbonitrile (D21)

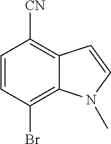

The mixture of 7-bromo-1H-indole-4-carbonitrile (300 mg), dimethyl carbonate (2445 mg) and DABCO (152 mg) in anhydrous DMF (9 mL) was heated up to 120° C. for 1 hour. After removing the solvent and excess dimethyl carbonate, the residue was dissolved in ethyl acetate and washed with water and brine. The solvent was evaporated to dryness and the residue was purified on ISCO eluting with 5% (EtOAc:Petroleum) to obtain 275 mg 7-bromo-1-methyl-1H-indole-4-carbonitrile (D21).

MS (ES): C₁₀H₇BrIN₂ requires 234.0 (⁷⁹Br), 236.1 (⁸¹Br); found 235.1 (M+H⁺, ⁷⁹Br), 237.2 (M+H⁺, ⁸¹Br).

Description for D22

### 7-Bromo-1-ethyl-1H-indole-4-carbonitrile (D22)

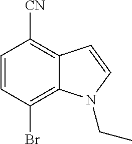

The mixture of 7-bromo-1H-indole-4-carbonitrile (250 mg), bromoethane (0.84 mL) and potassium hydroxide (190 mg) in dimethyl sulfoxide (4 mL) was stirred for 2 hours and the reaction was monitored by LCMS showing no starting material left. The mixture was poured into water and extracted with methylene chloride and dried over sodium sulphate. After removing the solvent, the residue was purified by ISCO (EtOAc:Petroleum=15%) obtaining the 260 mg 7-bromo-1-ethyl-1H-indole-4-carbonitrile as a solid.

MS (ES): C₁₁H₉BrN₂ requires 248.0 (⁷⁹Br), 250.1 (⁸¹Br); found 249.1 (M+H⁺, ⁷⁹Br), 251.2 (M+H⁺, ⁸¹Br).

Description for D23

### 7-Bromo-1-[(4-methylphenyl)sulfonyl]-1H-indole-4-carbonitrile (D23)

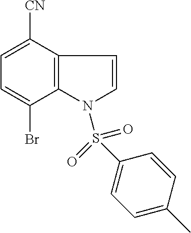

Under nitrogen, 0-5° C., sodium hydride (98 mg) was added to the solution of 7-bromo-1H-indole-4-carbonitrile (300 mg) in anhydrous DMF (8 mL). After stirring for 10 min, TsCl (517 mg) was added to the mixture. The mixture was stirred for 2 hours and monitored by LCMS showing no SM left and desired product was produced. After poring to the ice-water, the mixture was extracted with EtOAc and the organic layer was washed with brine, dry over sodium sulphate, evaporated under reduced pressure. The residue was purified by ISCO eluting with 10% EtOAc;Petroleum obtaining the product 440 mg 7-bromo-1-[(4-methylphenyl)sulfonyl]-1H-indole-4-carbonitrile (D23).

MS (ES): C₁₆H₁₁BrN₂O₂S requires 374.1 (⁷⁹Br), 376.0 (⁸¹Br); found 375.0 (M+1, ⁷⁹Br), 377.1 (M+1, ⁸¹Br).

Description for D24

### Ethyl 3-(4-cyano-1-methyl-1H-indol-7-yl)propanoate (D24)

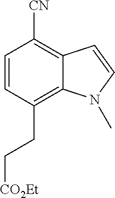

Under nitrogen atmosphere, the mixture of 7-bromo-1-methyl-1H-indole-4-carbonitrile (201 mg), Pd₂(dba)₃ (156 mg), P(tBu)₃HBF₄ (49 mg), Cs₂CO₃ (111 mg) and bromo[3-(ethyloxy)-3-oxopropyl]zinc (1053 mg) (0.5 M in THF) in anhydrous THF (6 mL) was heated up to 110° C. (oil bath) for 1 hr. The reaction was monitored by LCMS. After the starting material was disappeared, the reaction mixture was cooled to RT, poured to water, extracted with EtOAc. The organic layer was washed with brine and dried over sodium sulphate, evaporated under reduced pressure. The residue was purified on ISCO using 12 g silica gel column eluting with 30% (EtOAC/Petroleum) to obtain the product 180 mg ethyl 3-(4-cyano-1-methyl-1H-indol-7-yl)propanoate (D24).

MS (ES): C₁₅H₁₆IN₂O₂ requires 256.1; found 257.2 (M+H⁺).

Description for D25

### Ethyl 4-(4-cyano-1-methyl-1H-indol-7-yl)butanoate D25

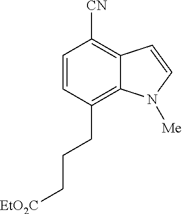

Under nitrogen atmosphere, bromo[4-(ethyloxy)-4-oxobutyl]zinc (914 mg) was added to the mixture of 7-bromo-1-methyl-1H-indole-4-carbonitrile (275 mg), Pd₂(dba)₃ (214 mg), P(tBu)₃HBF₄ (67.8 mg) and Cs₂CO₃ (152 mg) in anhydrous THF (10 mL). The resulting mixture was heated up to 80° C. and stirred at this temperature for 30 min. The reaction was monitored by LCMS showing no starting material left. After cooled to RT, the mixture was filtered to remove the palladium and base, and water was added to the filtration. The mixture was extracted with ethyl acetate, the organic layer was washed with brine, dried over sodium sulphate, evaporated under reduce pressure, the residue was purified by ISCO on silica gel eluting with 20% EtOAc/Petroleum obtaining 305 mg ethyl 4-(4-cyano-1-methyl-1H-indol-7-yl)butanoate (D25).

MS (ES): C₁₆H₁₈IN₂O₂ requires 270.1; found 271.2 (M+H⁺).

Description for D26

### Ethyl 3-{4-cyano-1-[(4-methylphenyl)sulfonyl]-1H-indol-7-yl}propanoate (D26)

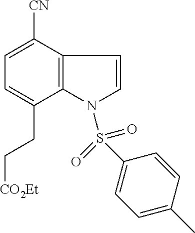

Under nitrogen, RT, bromo[3-(ethyloxy)-3-oxopropyl]zinc (788 mg) was added to the mixture of 7-bromo-1-[(4-methylphenyl)sulfonyl]-1H-indole-4-carbonitrile (400 mg), Pd₂(dba)₃ (195 mg), P(tBu)₃HBF₄ (62 mg) and Cs₂CO₃ (139 mg) in anhydrous THF (10 mL). The resulting mixture was heated up to reflux for 1 hr. The reaction was monitored by LCMS showing no starting material left. The palladium was removed by filtration, and THF was removed under reduced pressure. The residue was dissolved in EtOAc and washed with brine, dried over sodium sulphate, evaporated to dryness and the residue was purified by ISCO eluting with 10%-20% EtOAc/Petroleum to obtain the product 268 mg ethyl 3-{4-cyano-1-[(4-methylphenyl)sulfonyl]-1H-indol-7-yl}propanoate (D26).

MS (ES): C₂₁H₂₀N₂O₄S requires 396.1; found 397.1 (M+1).

Description for D27

### Ethyl 4-{4-cyano-1-[(4-methylphenyl)sulfonyl]-1H-indol-7-yl}butanoate (D27)

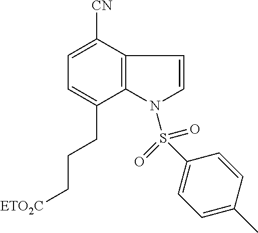

Under nitrogen, RT, bromo[4-(ethyloxy)-4-oxobutyl]zinc (1041 mg) was added to the mixture of 7-bromo-1-[(4-methylphenyl)sulfonyl]-1H-indole-4-carbonitrile (500 mg), Pd₂(dba)₃ (244 mg), P(tBu)₃HBF₄ (77 mg) and Cs₂CO₃ (173 mg) in anhydrous THF (10 mL). The resulting mixture was heated up to reflux for 30 min. The reaction was monitored by LCMS showing no starting material left. The palladium was removed by filtration, and THF was removed under reduced pressure. The residue was dissolved in EtOAc and washed with brine, dried over sodium sulphate, evaporated to dryness and the residue was purified by ISCO eluting with 10%-20% EtOAc/Petroleum to obtain the product 290 mg ethyl 4-{4-cyano-1-[(4-methylphenyl)sulfonyl]-1H-indol-7-yl}butanoate (D27).

MS (ES): C₂₂H₂₂N₂O₄S requires 410; found 411.1 (M+1).

Description for D28

### 7-Bromo-1-ethyl-N-hydroxy-1H-indole-4-carboximidamide (D28)

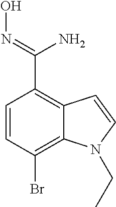

The mixture of 7-bromo-1-ethyl-1H-indole-4-carbonitrile (260 mg), hydroxylamine hydrochloride (145 mg) and sodium bicarbonate (263 mg) in ethanol (20 mL) was heated up to reflux and stirred for 25 hours, one pot additional hydroxylamine hydrochloride (145 mg) and sodium bicarbonate (263 mg) was added. The reaction was monitored by LCMS. The reaction mixture was cooled to RT and the solid was removed by filtration and washed successfully with ethanol and THF. The filtration was evaporated under reduced pressure to obtain the crude product 7-bromo-1-ethyl-N-hydroxy-1H-indole-4-carboximidamide (D28) it was used in the next step without further purification.

Description for D29

### Ethyl 3-{4-[(Z)-(hydroxyamino)(imino)methyl]-1H-indol-7-yl}propanoate (D29)

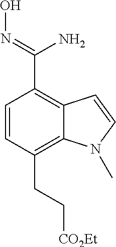

The mixture of ethyl 3-(4-cyano-1-methyl-1H-indol-7-yl)propanoate (180 mg), hydroxylamine hydrochloride (98 mg) and sodium bicarbonate (177 mg) in Ethanol (20 mL) was heated up to reflux and stirred for 25 hours, two pot additional hydroxylamine hydrochloride (98 mg) and sodium bicarbonate (177 mg) was added. The reaction was monitored by LCMS. The reaction mixture was cooled to RT and the solid was removed by filtration and washed successfully with ethanol and THF. The filtration was evaporated under reduced pressure to obtain the crude product ethyl 3-{4-[(Z)-(hydroxyamino)(imino)methyl]-1-methyl-1H-indol-7-yl}propanoate (D29), it was used in the next step without further purification.

Description for D30

### Ethyl 4-{4-[(Z)-(hydroxyamino)(imino)methyl]-1-methyl-1H-indol-7-yl}butanoate (D30)

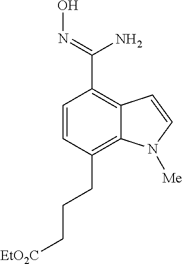

The mixture of ethyl 4-(4-cyano-1-methyl-1H-indol-7-yl)butanoate (316 mg), hydroxylamine hydrochloride (162 mg) and sodium bicarbonate (295 mg) in ethanol (10 mL) was heated up to reflux for 6 hours. The reaction was cooled to RT, after removing the solid by filtration, the solvent was evaporated under reduced pressure, the residue was purified by ISCO eluting with 50% EtOAc:Petroleum, the product ethyl 4-{4-[(Z)-(hydroxyamino)(imino)methyl]-1-methyl-1H-indol-7-yl}butanoate (D30) was obtained.

Description for D31

### Ethyl 3-{4-[(Z)-(hydroxyamino)(imino)methyl]-1-[(4-methylphenyl)sulfonyl]-1H-indol-7-yl}propanoate (D31)

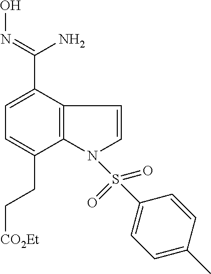

The mixture of ethyl 3-{4-cyano-1-[(4-methylphenyl)sulfonyl]-1H-indol-7-yl}propanoate (268 mg), hydroxylamine hydrochloride (94 mg) and sodium bicarbonate (170 mg) in ethanol (10 mL) was heated up to reflux for 19 hours. The reaction was cooled to RT, after removing the solid by filtration, the solvent was evaporated under reduced pressure, the residue was purified by ISCO eluting with 50% EtOAc:Petroleum to obtain 202 mg ethyl 3-{4-[(Z)-(hydroxyamino)(imino)methyl]-1-[(4-methylphenyl)sulfonyl]-1H-indol-7-yl}propanoate (D31).

MS (ES): C₂₁H₂₃N₃O₅S requires 429; found 430.1 (M+H⁺).

Description for D32

### Ethyl 4-{4-[(Z)-(hydroxyamino)(imino)methyl]-1-[(4-methylphenyl)sulfonyl]-1H-indol-7-yl}butanoate (D32)

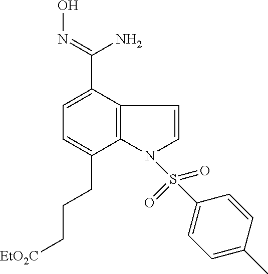

The mixture of ethyl 4-{4-cyano-1-[(4-methylphenyl)sulfonyl]-1H-indol-7-yl}butanoate (290 mg), hydroxylamine hydrochloride (196 mg) and sodium bicarbonate (297 mg) in ethanol (10 mL) was heated up to reflux for 8 hours. The reaction was cooled to RT, after removing the solid by filtration, the solvent was evaporated under reduced pressure, the residue was purified by ISCO eluting with 50% EtOAc:Petroleum, 210 mg ethyl 4-{4-[(Z)-(hydroxyamino)(imino)methyl]-1-[(4-methylphenyl)sulfonyl]-1H-indol-7-yl}butanoate (D32) was obtained.

MS (ES): C₂₂H₂₅N₃O₅S requires 443; found 444.2 (M+H⁺).

Description for D33

### 7-Bromo-4-(5-{3-chloro-4-[(1-methylethyl)oxy]phenyl}-1,2,4-oxadiazol-3-yl)-1-ethyl-1H-indole (D33)

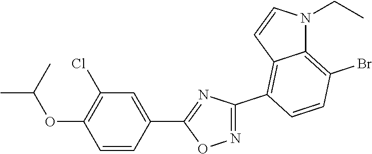

Under nitrogen atmosphere, the mixture of 3-chloro-4-[(1-methylethyl)oxy]benzoic acid (258 mg), EDC (288 mg), HOBT (230 mg) in THF (10 mL) was stirred at RT for 2 hours. 7-Bromo-1-ethyl-N-hydroxy-1H-indole-4-carboximidamide (282 mg) was added at RT. The mixture was stirred at RT for 3 hours. TBAF (784 mg) was added and the mixture was heated up to reflux for 48 hours. LCMS showed no starting material left. After cooled to RT, the mixture was partinationed to water and ethyl acetate. The organic layer was dried over sodium sulphate, evaporated under reduced pressure and the residue was purified by ISCO on silica gel eluting with methylene chloride to obtain the desired product 401 mg 7-bromo-4-(5-{3-chloro-4-[(1-methylethyl)oxy]phenyl}-1,2,4-oxadiazol-3-yl)-1-ethyl-1H-indole (D33).

MS (ES): C₂₁H₁₉BrCIN₃O₂ requires 459.0; found 460.1 (M+H⁺).

Description for D34

### Ethyl 3-[4-(5-{3-chloro-4-[(1-methylethyl)oxy]phenyl}-1,2,4-oxadiazol-3-yl)-1H-indol-7-yl]propanoate (D34)

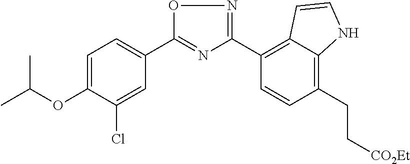

The mixture of 3-chloro-4-[(1-methylethyl)oxy]benzoic acid (60 mg), EDC (67 mg,) and HOBT (53 mg) was stirred at RT under nitrogen for 1 hour, and then ethyl 3-{4-[(Z)-(hydroxyamino)(imino)methyl]-1-[(4-methylphenyl)sulfonyl]-1H-indol-7-yl}propanoate (100 mg) was added and stirred for 1 hour, LCMS showing no starting material left. TBAF (183 mg) was added and the mixture was heated up to reflux for 48 hours. After cooled to RT, the mixture was partinationed to water and ethyl acetate. The organic layer was dried over sodium sulphate, evaporated under reduced pressure and the residue was purified by ISCO on silica gel eluting with 15% EtOAc/Petroleum to obtain 65 mg ethyl 3-[4-(5-{3-chloro-4-[(1-methylethyl)oxy]phenyl}-1,2,4-oxadiazol-3-yl)-1H-indol-7-yl]propanoate (D34).

MS (ES): C₂₄H₂₄CIN₃O₄ requires 453.1; found 454.2 (M+H⁺).

Description for D35

### Ethyl 3-[4-(5-{3-chloro-4-[(1-methylethyl)oxy]phenyl}-1,2,4-oxadiazol-3-yl)-1-methyl-1H-indol-7-yl]propanoate (D35)

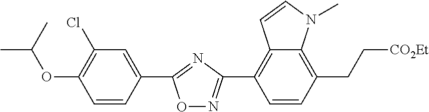

Under nitrogen atmosphere, the mixture of 3-chloro-4-[(1-methylethyl)oxy]benzoic acid (89 mg), EDC (99 mg), HOBT (79 mg), and ethyl 3-{4-[(Z)-(hydroxyamino)(imino)methyl]-1-methyl-1H-indol-7-yl}propanoate (70 mg) in THF (10 mL) was stirred at RT for 12 hours. TBAF (271 mg) was added and the mixture was heated up to reflux for 48 hours. LCMS showed no starting material left. After cooled to RT, the mixture was partinationed to water and ethyl acetate. The organic layer was dried over sodium sulphate, evaporated under reduced pressure and the residue was purified by ISCO on silica gel eluting with methylene chloride to obtain the desired product 65 mg ethyl 3-[4-(5-{3-chloro-4-[(1-methylethyl)oxy]phenyl}-1,2,4-oxadiazol-3-yl)-1-methyl-1H-indol-7-yl]propanoate (D35).

MS (ES): C₂₅H₂₆CIN₃O₄ requires 467.1; found 468.2 (M+H⁺).

Description for D36

### Ethyl 3-[4-(5-{3-chloro-4-[(1-methylethyl)oxy]phenyl}-1,2,4-oxadiazol-3-yl)-1-ethyl-1H-indol-7-yl]propanoate (D36)

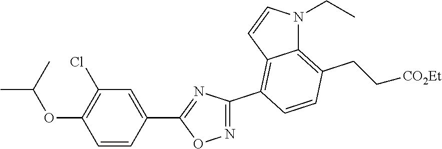

Under nitrogen, bromo[3-(ethyloxy)-3-oxopropyl]zinc (267 mg) was added to the mixture of 7-bromo-4-(5-{3-chloro-4-[(1-methylethyl)oxy]phenyl}-1,2,4-oxadiazol-3-yl)-1-ethyl-1H-indole (100 mg), Pd₂(dba)₃ (39 mg), P(tBu)₃HBF₄ (12 mg) and Cs₂CO₃ (28 mg) in anhydrous THF (5 mL). The resulting mixture was heated up to 80° C. and stirred at this temperature for 1 hour. The reaction was monitored by LCMS showing no starting material left. After cooled to RT, the mixture was filtered to remove the palladium and base, and water was added to the filtration. The mixture was extracted with ethyl acetate, the organic layer was washed with brine, dried over sodium sulphate, evaporated under reduce pressure, the residue was purified by ISCO on silica gel column eluting with 20% EtOAc/Petroleum obtaining 93 mg ethyl 3-[4-(5-{3-chloro-4-[(1-methylethyl)oxy]phenyl}-1,2,4-oxadiazol-3-yl)-1-ethyl-1H-indol-7-yl]propanoate (D36).

MS (ES): C₂₆H₂₈CIN₃O₄ requires 481.1; found 482.2 (M+H⁺).

Description for D37

### Ethyl 4-[4-(5-{3-chloro-4-[(1-methylethyl)oxy]phenyl}-1,2,4-oxadiazol-3-yl)-1H-indol-7-yl]butanoate (D37)

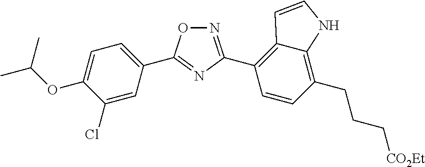

EDC (64 mg) and HOBT (51 mg) was added to the solution of 3-chloro-4-[(1-methylethyl)oxy]benzoic acid (58 mg) in THF (10 mL). After stirring for 1 hour, ethyl 4-{4-[(Z)-(hydroxyamino)(imino)methyl]-1-[(4-methylphenyl)sulfonyl]-1H-indol-7-yl}butanoate (100 mg) was added. After stirring for 1 hour, TBAF (295 mg) was added and the mixture was stirred at refluxing overnight. The reaction was monitored by LCMS. After cooling to RT, the mixture was directly loaded on silica gel column and purified by ISCO eluting with 30% EtOAc/Petroleum to obtain the desired product 70 mg ethyl 4-[4-(5-{3-chloro-4-[(1-methylethyl)oxy]phenyl}-1,2,4-oxadiazol-3-yl)-1H-indol-7-yl]butanoate (D37).

MS (ES): C₂₅H₂₆CIN₃O₄ requires 467.1; found 468.2 (M+H⁺).

Description for D38

### Ethyl 4-[4-(5-{3-chloro-4-[(1-methylethyl)oxy]phenyl}-1,2,4-oxadiazol-3-yl)-1-methyl-1H-indol-7-yl]butanoate (D38)

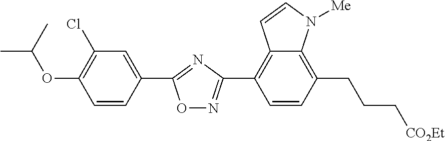

Under nitrogen atmosphere, the mixture of 3-chloro-4-[(1-methylethyl)oxy]benzoic acid (67 mg), EDC (76 mg) and HOBT (60 mg) in anhydrous THF (5 mL) was stirred at RT for 2 hours. Then ethyl 4-{4-[(Z)-(hydroxyamino)(imino)methyl]-1-methyl-1H-indol-7-yl}butanoate (80 mg) was added and the mixture was stirred at RT for 2 hours, monitored by LCMS. TBAF (207 mg) was added and the mixture was heated up to reflux for 24 hours. LCMS showed no starting material left. After cooled to RT, the mixture was partinationed to water and ethyl acetate. The organic layer was dried over sodium sulphate, evaporated under reduced pressure and the residue was purified by ISCO on silica gel eluting with methylene chloride to obtain 100 mg ethyl 4-[4-(5-{3-chloro-4-[(1-methylethyl)oxy]phenyl}-1,2,4-oxadiazol-3-yl)-1-methyl-1H-indol-7-yl]butanoate (D38) as a white solid.

MS (ES): C₂₆H₂₈CIN₃O₄ requires 481.1; found 482.2 (M+H⁺).

Description for D39

### Ethyl 4-[4-(5-{3-chloro-4-[(1-methylethyl)oxy]phenyl}-1,2,4-oxadiazol-3-yl)-1-ethyl-1H-indol-7-yl]butanoate (D39)

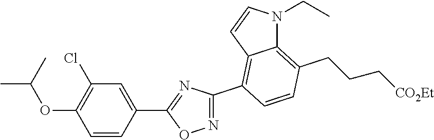

Under nitrogen, bromo[4-(ethyloxy)-4-oxobutyl]zinc (283 mg) was added to the mixture of 7-bromo-4-(5-{3-chloro-4-[(1-methylethyl)oxy]phenyl}-1,2,4-oxadiazol-3-yl)-1-ethyl-1H-indole (100 mg), Pd₂(dba)₃ (39 mg), P(tBu)₃HBF₄ (12 mg) and Cs₂CO₃ (28 mg) in anhydrous THF (5 mL). The resulting mixture was heated up to 80° C. and stirred at this temperature for 1 hour. The reaction was monitored by LCMS showing no starting material left. After cooled to RT, the mixture was filtered to remove the palladium and base, and water was added to the filtration. The mixture was extracted with ethyl acetate, the organic layer was washed with brine, dried over sodium sulphate, evaporated under reduce pressure, the residue was purified by ISCO on silica gel column eluting with 20% EtOAc/Petroleum obtaining 75 mg product ethyl 4-[4-(5-{3-chloro-4-[(1-methylethyl)oxy]phenyl}-1,2,4-oxadiazol-3-yl)-1-ethyl-1H-indol-7-yl]butanoate (D39).

MS (ES): C₂₇H₃₀CIN₃O₄ requires 495.1; found 496.2 (M+H⁺).

Description for D40

### Methyl 3-[4-(5-{5-chloro-6-[(1-methylethyl)oxy]-3-pyridinyl}-1,2,4-oxadiazol-3-yl)-1-methyl-1H-indol-7-yl]propanoate (D40)

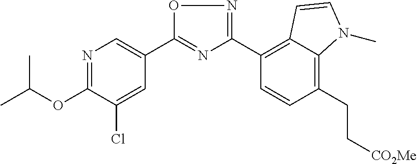

The mixture of 3-[4-(5-{5-chloro-6-[(1-methylethyl)oxy]-3-pyridinyl}-1,2,4-oxadiazol-3-yl)-1H-indol-7-yl]propanoic acid (70 mg), dimethyl carbonate (295 mg) and DABCO (27 mg) in DMF (5 mL) was heated up to 100° C. and stirring at this temperature for 3 hours. The reaction mixture was quenched with 30 mL water, extracted with EtOAc (30 mL×3). The organic phase was washed with saturated brine 50 mL, dried over sodium sulphate and evaporated in vacuo to give the crude product as a yellow oil. The crude product was added to a Biotage column and was eluted with 50% to obtain the product 55 mg methyl 3-[4-(5-{5-chloro-6-[(1-methylethyl)oxy]-3-Pyridinyl}-1,2,4-oxadiazol-3-yl)-1-methyl-1H-indol-7-yl]propanoate (D40).

MS (ES): C₂₃H₂₃CIN₄O₄ requires 454.1; found 455.2 (M+H⁺).

Description for D41

### Ethyl 3-[4-(5-{5-chloro-6-[(1-methylethyl)oxy]-3-pyridinyl}-1,2,4-oxadiazol-3-yl)-1-ethyl-1H-indol-7-yl]propanoate (D41)

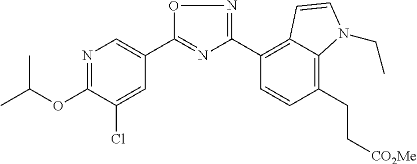

The mixture of 3-[4-(5-{5-chloro-6-[(1-methylethyl)oxy]-3-pyridinyl}-1,2,4-oxadiazol-3-yl)-1H-indol-7-yl]propanoic acid (70 mg), bromoethane (0.122 mL) and solid potassium hydroxide (46 mg) in dimethyl sulfoxide (2 mL) was stirred at RT overnight. The reaction was quenched with water, extracted with EtOAc, washed with brine, dried over sodium sulphate, evaporated to dryness, and used in next step without further purification.

Description for D42

### Ethyl 4-[4-(5-{5-chloro-6-[(1-methylethyl)oxy]-3-pyridinyl)-1,2,4-oxadiazol-3-yl)-1H-indol-7-yl]butanoate (D42)

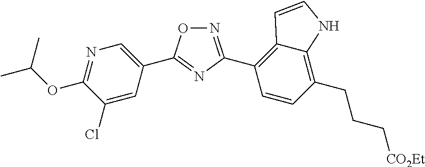

EDC (123 mg) and HOBT (98 mg) was added to the solution of 5-chloro-6-[(1-methylethyl)oxy]-3-pyridinecarboxylic acid (111 mg) in THF (10 mL). After stirring for 30 min, ethyl 4-{4-[(Z)-(hydroxyamino)(imino)methyl]-1-[(4-methylphenyl)sulfonyl] -1H-indol-7-yl}butanoate (190 mg) was added and stirring for 1 hour. TBAF (336 mg) was added and the mixture was stirred at refluxing overnight. The reaction was monitored by LCMS. After cooling to RT, the mixture was directly loaded on silica gel column and purified by ISCO eluting with 30% EtOAc/Petroleum to obtain the desired product 150 mg ethyl 4-[4-(5-{5-chloro-6-[(1-methylethyl)oxy]-3-pyridinyl}-1,2,4-oxadiazol-3-yl)-1H-indol-7-yl]butanoate (D42).

MS (ES): C₂₄H₂₅CIN₄O₄ requires 468.1; found 469.2 (M+H⁺).

Description for D43

### Ethyl 4-[4-(5-{5-chloro-6-[(1-methylethyl)oxy]-3-pyridinyl}-1,2,4-oxadiazol-3-yl)-1-methyl-1H-indol-7-yl]butanoate (D43)

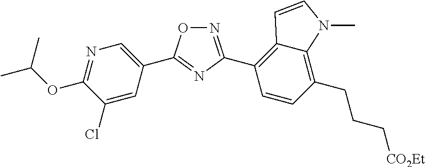

The mixture of 5-chloro-6-[(1-methylethyl)oxy]-3-pyridinecarboxylic acid (109 mg), EDC (121 mg) and HOBT (97 mg) in THF stirred at RT for 2 hours, then ethyl 4-{4-[(Z)-(hydroxyamino)(imino)methyl]-1-methyl-1H-indol-7-yl}butanoate (128 mg) was added to the mixture in one pot. After the resulting mixture was stirred at RT for 2 hours, TBAF (331 mg) was added and the mixture was heated up to reflux overnight. After cooled to RT, the mixture was partinationed to water and ethyl acetate. The organic layer was dried over sodium sulphate, evaporated under reduced pressure and the residue was purified by ISCO on silica gel eluting with 15% EtOAc/Petroleum to obtain the desired product 180 mg ethyl 4-[4-(5-{5-chloro-6-[(1-methylethyl)oxy]-3-pyridinyl}-1,2,4-oxadiazol-3-yl)-1-methyl-1H-indol-7-yl]butanoate (D43).

MS (ES): C₂₅H₂₇CIN₄O₄ requires 482.1; found 483.2 (M+H⁺).

Description for D44

### Ethyl 4-[4-(5-{3-chloro-4-[(1-methylethyl)oxy]phenyl}-1,2,4-oxadiazol-3-yl)-1-ethyl-1H-indol-7-yl]butanoate (D44)

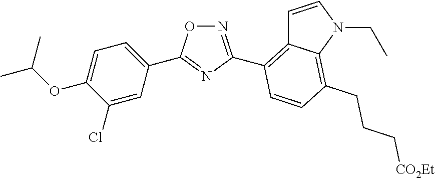

Under nitrogen, bromo[4-(ethyloxy)-4-oxobutyl]zinc (283 mg) was added to the mixture of 7-bromo-4-(5-{3-chloro-4-[(1-methylethyl)oxy]phenyl}-1,2,4-oxadiazol-3-yl)-1-ethyl-1H-indole (100 mg), Pd₂(dba)₃ (39 mg), P(tBu)₃HBF₄ (12 mg) and Cs₂CO₃ (28 mg) in anhydrous THF (5 mL). The resulting mixture was heated up to 80° C. and stirred at this temperature for 1 hour. The reaction was monitored by LCMS showing no starting material left. After cooled to RT, the mixture was filtered to remove the palladium and base, and water was added to the filtration. The mixture was extracted with ethyl acetate, the organic layer was washed with brine, dried over sodium sulphate, evaporated under reduce pressure, the residue was purified by ISCO on silica gel eluting with 20% EtOAc/Petroleum obtaining 75 mg product ethyl 4-[4-(5-{3-chloro-4-[(1-methylethyl)oxy]phenyl}-1,2,4-oxadiazol-3-yl)-1-ethyl-1H-indol-7-yl]butanoate (D44).

MS (ES): C₂₇H₃₀CIN₃O₄ requires 495.1; found 496.2 (M+H⁺).

Description for D45

### Ethyl 3-[4-(5-{3-cyano-4-[(1-methylethyl)oxy]phenyl} -1,2,4-oxadiazol-3-yl)-1H-indol-7-yl]propanoate (D45)

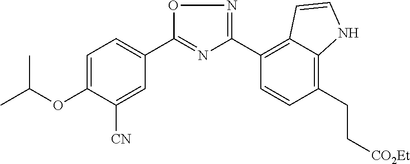

The mixture of 3-cyano-4-[(1-methylethyl)oxy]benzoic acid (235 mg), EDC (275 mg) and HOBT (219 mg) was stirred at RT under nitrogen for 1 hour, and then ethyl 3-{4-[(Z)-(hydroxyamino)(imino)methyl]-1-[(4-methylphenyl)sulfonyl]-1H-indol-7-yl}propanoate (410 mg) was added and stirred for 1 hour, LCMS showed no starting material left. TBAF (749 mg) was added and the mixture was heated up on Biotage microwave at 120° C. for 1.5 hours. After cooled to RT, the mixture was paRTinationed to water and ethyl acetate. The organic layer was dried over sodium sulphate, evaporated under reduced pressure and the residue was purified by ISCO on silica gel eluting with 15% EtOAc/Petroleum to obtain the desired product 230 mg ethyl 3-[4-(5-{3-cyano-4-[(1-methylethyl)oxy]phenyl}-1,2,4-oxadiazol-3-yl)-1H-indol-7-yl]propanoate (D45).

MS (ES): C₂₅H₂₄N₄O₄ requires 444.1; found 445.2 (M+H⁺).

Description for D46

### 2-Bromo-5-methoxy-phenol (D46)

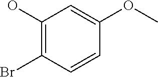

To a mixture of 3-methoxy-phenol (30 g) in DCM (700 mL) was added NBS (43 g). The mixture was stirred and refluxed overnight. TLC showed the complete consumption of 3-Methoxy-phenol, and the mixture was quenched by water. The organic layer was washed with water, evaporated to give the crude product, which was purified by column chromatography on silica gel eluted with (EA: PE=1:100) to obtain 2-Bromo-5-methoxy-phenol (D46) (33.5 g).

MS (ES): C₇H₇BrO₂ requires 203; found 204.1.2 (M+H⁺).

Description for D47

### 1-Bromo-2-(2,2-diethoxy-ethoxy)-4-methoxy-benzene (D47)

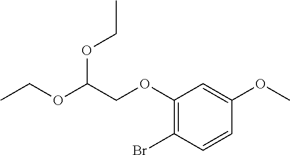

To a mixture of 2-bromo-5-methoxy-phenol (23.5 g), K₂CO₃ (24 g) and DMF was added 2-bromo-1,1-diethoxy-ethane dropwise (27.3 g) at 0° C. Then the mixture was heated at 120° C. overnight. The mixture was cooled, diluted with EA, washed with water. The organic layer was evaporated to give 1-bromo-2-(2,2-diethoxy-ethoxy)-4-methoxy-benzene (43 g, crude) without further purification for next step.

MS (ES): C₁₃H₁₉BrO₄ requires 319; found 342.1.2 (M+Na⁺).

Description for D48

### 7-Bromo-4-methoxy-benzofuran (D48)

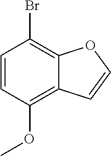

To a mixture of PPA (54 g) and chlorobenzene (200 mL) was added dropwise a solution of 1-bromo-2-(2,2-diethoxy-ethoxy)-4-methoxy-benzene (51 g) in chlorobenzene (100 mL) at 80° C. The mixture was stirred at 80° C. overnight, then heated to 120° C. for 2 hours. The mixture was cooled, neutralized with NaOH (2N), diluted with Et₂O. The organic layer was evaporated to give the crude product, which was purified by column chromatography on silica gel eluted with (EA: PE=1:200 to 1:100 to 1:50) to give 7-Bromo-4-methoxy-benzofuran (D48) (25.4 g).

MS (ES): C₉H₇BrO₂ requires 227; found 228.1 (M+H⁺).

Description for D49

### 4-Methoxy-benzofuran-7-carbonitrile (D49)

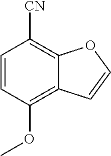

To a mixture of 7-Bromo-4-methoxy-benzofuran (25.4 g), Zn(CN)₂ (52 g) and DMF (800 mL) was added Pd(PPh₃)₄ (25.4 g) under N₂. The mixture was heated at 80° C. overnight. Then the mixture was cooled, filtered, the solvent was evaporated, the residue was purified by column chromatography on silica gel eluted with (EA: PE=1:200 to 1:100 to 1:50) to give 4-Methoxy-benzofuran-7-carbonitrile (D49) (10.0 g).

MS (ES): C₁₀H₇NO₂ requires 173.1; found 174.2 (M+H⁺).

Description for D50

### 4-Hydroxy-benzofuran-7-carbonitrile (D50)

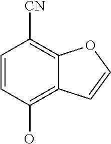

To a mixture of 4-Methoxy-benzofuran-7-carbonitrile (10 g) in DCM (800 mL) was added dropwise BBr₃ (116 mL 1.0 M in DCM) at −78° C., then stirred at room temperature for one day. Additional four batches of BBr₃ (116 mL 1.0 M in DCM) was added to the mixture in four days respectively. TLC showed the complete consumption of 4-Methoxy-benzofuran-7-carbonitrile, the mixture was quenched by adding with water at −78° C. The organic layer was evaporated, the residue was recrystallized from (EA: PE=1:20) to give 4-Hydroxy-benzofuran-7-carbonitrile (D50) (5.33 g).

δH (DMSO-d₆, 400 MHz): 6.73 (1H, d), 7.08 (1H, d), 7.61 (1H, d), 8.02 (1H, d), 11.27 (1H, s). MS (ES): C₉H₅NO₂ requires 159.1; found 160.2 (M+H⁺).

Description for D51

### 2-(2,2-Diethoxyethoxy)-4-bromo-1-methoxybenzene (D51)

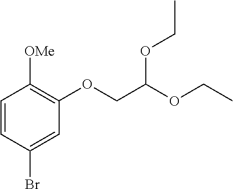

To a suspension of 60% of NaH/mineral (11.8 g) in DMF (200 mL) under nitrogen atmosphere was added 5-bromo-2-methoxyphenol (50.0 g) in portions at 0° C., carefully controlled the hydrogen gas. After being stirred for 30 min, (2-bromo-1-ethoxyethoxy)ethane (72.7 g) was added and the reaction was allowed to heat to reflux for 6 hrs. TLC indicated the reaction completed. The reaction mixture was cooled to room temperature, poured into ice-water, and extracted with EtOAc. The organic layer was dried over anhydrous Na₂SO₄, filtered, and concentrated to afford the crude product. Purification was performed by a silica gel column to afford desired product (D51) as yellow oil (69.1 g).

δH (CDCl₃, 400 MHz): 1.21 (6H, m), 3.58 (4H, m), 3.72 (3H, s), 4.82 (1H, m), 6.69 (1H, d), 7.02 (2H, m).

Description for D52

### 4-Bromo-7-methoxybenzofuran (D52)

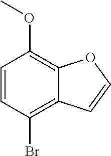

PPA (79.5 g) and chlorobenzene (60 mL) were combined and heated to reflux. To the refluxing mixture was added 2-(2,2-diethoxyethoxy)-4-bromo-1-methoxybenzene (50.0 g) dropwise, which was dissolved in chlorobenzene (60 mL) over 50 minutes. Heating of the reaction mixture at reflux was continued for 2 hours and then it was cooled to room temperature. 1N NaOH was added and the reaction was stirred at room temperature overnight. The reaction mixture was then extracted with EtOAc, the organic layer were dried over anhydrous Na₂SO₄, filtered and concentrated under vacuum. The residue was purified by flash chromatography to provide the desired product (D52) as clear oil (30.6 g).

δH (CDCl₃, 400 MHz): 3.97 (3H, s), 6.67 (1H, d), 6.78 (2H, d), 7.30 (1H, d), 7.64 (1H, d).

Description for D53

### 7-Methoxybenzofuran-4-carbonitrile (D53)

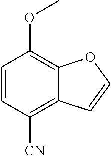

A mixture of 4-bromo-7-methoxybenzofuran (45.0 g), Zn(CN)₂ (17.4 g), Pd(PPh₃)₄ (22.9 g) and DMF (150 mL) was stirred at 100° C. for 4 hours under N₂ atmosphere. After cooling to room temperature, the reactant was poured into water and extracted with EtOAc. Insoluble solid were filtered off using celite. The organic layer was washed with brine, dried over Na₂SO₄, and concentrated. The residue was purified by a silica gel column chromatography to obtain the desired product as a white solid (D53) (19.17 g).

δH (CDCl₃, 400 MHz): 4.05 (3H, s), 6.81 (1H, d), 6.95 (1H, d), 7.52 (1H, d), 7.73 (1H, s)

Description for D54

### 7-Hydroxybenzofuran-4-carbonitrile (D54)

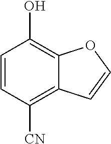

To a solution of 7-methoxybenzofuran-4-carbonitrile (10.0 g) in 35 mL DCM was added BBr₃ in 40 mL of DCM (1.0 M) drop wise at −78° C. The reaction mixture was then warmed to room temperature and stirred for 72 hours. The reaction was then added with water at 0° C., washed with 1N NaOH. The organic layer was dried over anhydrous Na₂SO₄, filtered, and concentrated to afford the crude product. Purification was performed by a silica gel column to give the pure product as a white solid (D54) (7.64 g).

δH (DMSO-d₆, 400 MHz): 6.85 (1H, m), 7.07(1H, d), 7.57 (1H, m), 8.19(1H, d), 11.36 (1H, s, br,).

Description for D55

### 4-[({[2-(Trimethylsilyl)ethyl]oxy}methyl)oxy]-1-benzofuran-7-carbonitrile (D55)

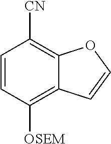

Under nitrogen atmosphere, SEMCI (0.368 mL) was added drop wise to the solution of 4-hydroxy-1-benzofuran-7-carbonitrile (300 mg) and DIEA (0.823 mL) in DCM (20 mL). The mixture was stirred at RT for 2 hours and LCMS showed no starting material left. The mixture was washed with water and brine, dried over sodium sulphate. After removing the solvent under reduced pressure, the residue was purified on silica gel column by ISCO eluting with 10% EtOAC/Petroleum to obtain 508 mg 4-[({[2-(trimethylsilyl)ethyl]oxy}methyl)oxy]-1-benzofuran-7-carbonitrile (D55).

MS (ES): C₁₅H₁₉NO₃Si requires 289.1; found 290.2 (M+H⁺).

Description for D56

### N-Hydroxy-4-[({[2-(trimethylsilyl)ethyl]oxy}methyl)oxy]-1-benzofuran-7-carboximidamide (D56)

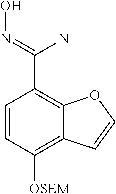

The mixture of 4-[({[2-(trimethylsilyl)ethyl]oxy}methyl)oxy]-1-benzofuran-7-carbonitrile (508 mg), hydroxylamine hydrochloride (488 mg) and sodium bicarbonate (885 mg) in ethanol (25 mL) was heated up to 80-90° C. and stirred at this temperature for 4 hours. Hydroxylamine hydrochloride and sodium bicarbonate was added in two ports every two hours. After removing the solid by filtration, the solvent was evaporated under reduced pressure. The residue was purified on silica gel column eluting with 40% EtOAc/Petroleum to obtain the product 540 mg N-hydroxy-4-[({[2-(trimethylsilypethyl]oxy}methyl)oxy]-1-benzofuran-7-carboximidamide (D56).

MS (ES): C₁₅H₂₂N₂O₄Si requires 322.1; found 323.2 (M+H⁺).

Description for D57

### 5-{3-Chloro-4-[(1-methylethyl)oxy]phenyl)-3-{4-[({[2-(trimethylsilyl)ethyl]oxy}methyl)oxy]-1-benzofuran-7-yl}-1,2,4-oxadiazole (D57)

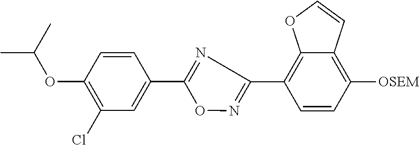

After the mixture of 3-chloro-4-[(1-methylethyl)oxy]benzoic acid (431 mg), EDC (482 mg) and HOBT (385 mg) was stirred for 1 hr at RT, N-hydroxy-4-[({[2-(trimethylsilyl)ethyl]oxy}methyl)oxy]-1-benzofuran-7-carboximidamide (540 mg) was added. The resulting mixture was stirred at RT overnight and LCMS showing no SM left. TBAF (1314 mg) was added and the mixture was heated up to reflux for 48 hours. The mixture was directly loaded on silica gel column eluting with 20% EtOAc/Petroleum to obtain 679 mg 5-{3-chloro-4-[(1-methylethyl)oxy]phenyl}-3-{4-[({[2-(trimethylsilyl)ethyl]oxy}methyl)oxy]-1-benzofuran-7-yl}-1,2,4-oxadiazole (D57).

MS (ES): C₂₅H₂₉CIN₂O₅Si requires 500.1; found 501.2 (M+H⁺).

Description for D58

### 7-(5-{3-Chloro-4-[(1-methylethyl)oxy]phenyl} -1,2,4-oxadiazol-3-yl)-1-benzofuran-4-ol (D58)

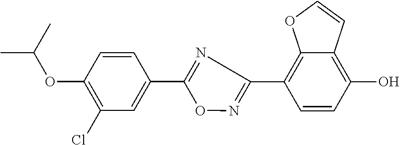

At RT, TBAF (354 mg) was added to the solution of 5-{3-chloro-4-[(1-methylethyl)oxy]phenyl}-3-{4-[({[2-(trimethylsilyl)ethyl]oxy}methyl)oxy]-1-benzofuran-7-yl}-1,2,4-oxadiazole (679 mg) in HMPA (10 mL). The resulting mixture was stirred at 60° C. for 30 min. After quenching the reaction with sodium bicarbonate saturated aq. and extracted with methylene chloride (30 mL×2). The organic phase was washed with brine 50 mL, dried over sodium sulphate and evaporated in vacuum to give the crude product as an off-white solid. The crude product was added to a Biotage column and was eluted with 70% Petroleum/EtOAc to obtain 260 mg 7-(5-{3-chloro-4-[(1-methylethyl)oxy]phenyl}-1,2,4-oxadiazol-3-yl)-1-benzofuran-4-ol (D58).

Description for D59

### Ethyl {[7-(5-{3-chloro-4-[(1-methylethyl)oxy]phenyl}-1,2,4-oxadiazol-3-yl)-1-benzofuran-4-yl]oxy}acetate (D59)

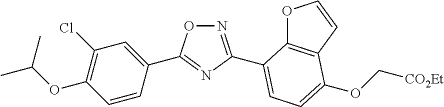

At rt, under nitrogen, ethyl bromoacetate (67 mg) and potassium carbonate (56 mg) was added to the solution of 7-(5-{3-chloro-4-[(1-methylethyl)oxy]phenyl}-1,2,4-oxadiazol-3-yl)-1-benzofuran-4-ol in anhydrous DMF (1.5 mL). The resulting mixture was stirred at RT for 2 hours. LCMS showed no starting material left. Due to using methanol as solvent for LCMS analysis, only the methyl ester showed up, but traces ethyl ester. 30 mL water was added to the system, extracted with EtOAc (50 mL×3).The organic phase was washed with saturated brine 50 mL, dried over sodium sulphate and evaporated in vacuo to give the crude product (D59) as a yellow oil, used in next step without further purification.

MS (ES): C₂₃H₂ᵢCIN₂O₆ requires 456.1; found 457.2 (M+H⁺).

Description for D60

### Ethyl 4-{[7-(5-{3-chloro-4-[(1-methylethyl)oxy]phenyl}-1,2,4-oxadiazol-3-yl)-1-benzofuran-4-yl]oxy}butanoate (D60)

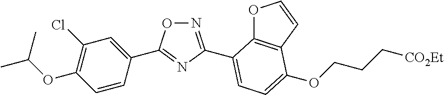

At rt, under nitrogen, ethyl 4-bromobutanoate (158 mg) and potassium carbonate (112 mg) was added to the solution of 7-(5-{3-chloro-4-[(1-methylethyl)oxy]phenyl}-1,2,4-oxadiazol-3-yl)-1-benzofuran-4-ol (100 mg) in anhydrous DMF (2 mL). The resulting mixture was stirred ar RT for 2 hours. LCMS showed no starting material left. Due to using methanol as solvent for LCMS analysis, not only the ethyl ester showed up, but also methyl ester.30 mL water was added to the system, extracted with EtOAc (50 mL×3).The organic phase was washed with saturated brine 50 mL, dried over sodium sulphate and evaporated in vacuum to give the crude product, purified on ISCO eluting with 30% EtOAc obtaining the product as a white solid 120 mg ethyl 4-{[7-(5-{3-chloro-4-[(1-methylethyl)oxy]phenyl}-1,2,4-oxadiazol-3-yl)-1-benzofuran-4-yl]oxy}butanoate (D60).

MS (ES): C₂₅H₂₅CIN₂O₆ requires 484.1; found 485.2 (M+H⁺).

Description for D61

### 7-[({(2-(Trimethylsilyl)ethyl]oxy}methyl)oxy]-1-benzofuran-4-carbonitrile (D61)

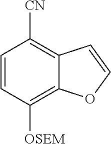

The mixture of 7-hydroxy-1-benzofuran-4-carbonitrile (2.0 g), N-ethyl-N-(1-methylethyl)-2-propanamineethyl[bis(1-methylethyl)]amine (2.4 g) in DCM (40 mL) was stirred at RT under nitrogen atmosphere. Then {2-[(chloromethyl)oxy]ethyl)(trimethyl)silane (2.3 g) was added dropwise. Aqueous NaHCO₃ (20 mL) was added after one hour. The organic layer was separated and dried over anhydrous magnesium sulfate. The dried solution was concentrated in vacuum to afford 7-[({[2-(trimethylsilyl)ethyl]oxy}methyl)oxy]-1-benzofuran-4-carbonitrile (D61) (3.6 g) as a yellow oil.

MS (ES): C₁₅H₁₉NO₃Si requires 289; found 290.1 (M+H⁺).

Description for D62

### N-Hydroxy-7-[({[2-(trimethylsilyl)ethyl]oxy}methyl)oxy]-1-benzofuran-4-carboximidamide (D62)

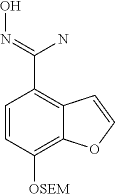

The mixture of 7-[({[2-(trimethylsilyl)ethyl]oxy}methyl)oxy]-1-benzofuran-4-carbonitrile (3.6 g), hydroxylamine hydrochloride (2.161 g), sodium bicarbonate (2.61 g) in ethanol (20 mL) was stirred at 85° C. for 5 hours. The solid was removed by filtration. The filtrate was concentrated in vacuum and the resulting residue was purified by Teledyne ISCO (30% EtOAc in Petroleum) to afford N-hydroxy-7-[({(2-(trimethylsilyl)ethylloxy}methyl)oxy]-1-benzofuran-4-carboximidamide (D62) (3.45 g) as a yellow solid.

MS (ES): C₁₅H₂₂N₂O₄Si requires 322; found 323.2 (M+H⁺).

Description for D63

### 5-{3-Chloro-4-[(1-methylethyl)oxy]phenyl}-3-{7-[({[2-(trimethylsilyl)ethyl]oxy}methyl)oxy]-1-benzofuran-4-yl)-1,2,4-oxadiazole (D63)

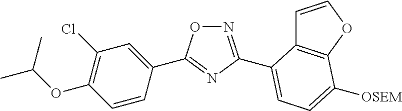

After the mixture of 3-chloro-4-[(1-methylethyl)oxy]benzoic acid (359 mg), EDC (401 mg) and HOBT (321 mg) was stirred for 1 hour at RT, N-hydroxy-7-[({[2-(trimethylsilyl)ethyl]oxy}methyl)oxy]-1-benzofuran-4-carboximidamide (450 mg) was added. The resulting mixture was stirred at RT for 2 hours and LCMS showing no SM left. TBAF (1095 mg) was added and the mixture was heated up to reflux for 48 hours. The mixture was directly loaded on silica gel column eluting with 20% EtOAc/Petroleum to obtain 442 mg 5-{3-chloro-4-[(1-methylethyl)oxy]phenyl}-3-{7-[({[2-(trimethylsilyl)ethyl]oxy}methyl)oxy]-1-benzofuran-4-yl}-1,2,4-oxadiazole (D63).

MS (ES): C₂₅H₂₉CIN₂O₅Si requires 500.1; found 501.2 (M+H⁺).

Description for D64

### 4-(5-{3-Chloro-4-[(1-methylethyl)oxy]phenyl}-1,2,4-oxadiazol-3-yl)-1-benzofuran-7-ol (D64)

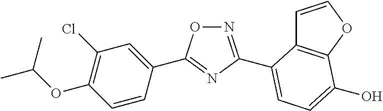

At rt, TBAF (130 mg) was added to the solution of 5-{3-chloro-4-[(1-methylethyl)oxy]phenyl}-3-{7-[({[2-(trimethylsilyl)ethyl]oxy}methyl)oxy]-1-benzofuran-4-yl}-1,2,4-oxadiazole (50 mg) in HMPA (5 mL). The resulting mixture was stirred at 50° C. for 30 min. After quenching the reaction with sodium bicarbonate saturated aq. and extracted with methylene chloride (30 mL×2). The organic phase was washed with brine 50 mL, dried over sodium sulphate and evaporated in vacuo to give the crude product as an oil. The crude product was added to a Biotage column and was eluted with 70% Petroleum/EtOAc to obtain 30 mg 4-(5-{3-chloro-4-[(1-methylethyl)oxy]phenyl}-1,2,4-oxadiazol-3-yl)-1-benzofuran-7-ol (D64).

MS (ES): C₁₉H₁₅CIN₂O₄ requires 370.1; found 371.2 (M+H⁺).

Description for D65

### Ethyl {(4-(5-{3-chloro-4-[(1-methylethyl)oxy]phenyl}-1,2,4-oxadiazol-3-yl)-1-benzofuran-7-yl]oxy}acetate (D65)

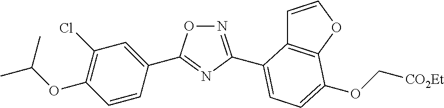

At RT, under nitrogen atmosphere, ethyl bromoacetate (54 mg) and potassium carbonate (44 mg) was added to the solution of 4-{5-[3-chloro-4-(ethyloxy)phenyl]-1,2,4-oxadiazol-3-yl}-1-benzofuran-7-ol (40 mg,) in anhydrous DMF (1.5 mL). The resulting mixture was stirred ar RT for 2 hours. LCMS showed no starting material left. Due to using methanol as solvent for LCMS analysis, the methyl ester mainly showed up, but only traces ethyl ester showed up. 30 mL water was added to the system, extracted with EtOAc (50 mL×3).The organic phase was washed with saturated brine 50 mL, dried over sodium sulphate and evaporated in vacuo to give the crude product (D65)as a yellow oil, used in next step without further purification.

Description for D66

### Ethyl 4-{[4-(5-(3-chloro-4-[(1-methylethyl)oxy]phenyl}-1,2,4-oxadiazol-3-yl)-1-benzofuran-7-yl]oxy}butanoate (D66)

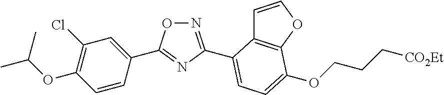

At rt, under nitrogen, ethyl 4-bromobutanoate (79 mg) and potassium carbonate (56 mg) was added to the solution of 4-{5-[3-chloro-4-(ethyloxy)phenyl]-1,2,4-oxadiazol-3-yl}-1-benzofuran-7-ol (50 mg) in anhydrous DMF (2 mL). The resulting mixture was stirred ar RT for 8 hours. The reaction is much slow in THF than in DMF. LCMS showed no starting material left. Due to using methanol as solvent for LCMS analysis, only the methyl ester showed up, but traces ethyl ester. 30 mL water was added to the system, extracted with EtOAc (50 mL×3).The organic phase was washed with saturated brine 50 mL, dried over sodium sulphate and evaporated in vacuum to give the crude product (D66) as a yellow oil, used in next step without further purification.

Description for D67

### N-[({5-Chloro-6-[(1-methylethyl)oxy]-3-pyridinyl}carbonyl)oxy]-7-[({[2-(trimethylsilyl)ethyl]oxy}methyl)oxy]-1-benzofuran-4-carboximidamide (D67)

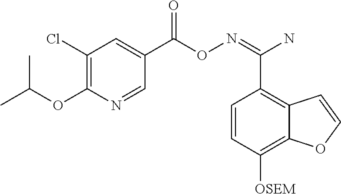

The mixture of N-hydroxy-7-[({[2-(trimethylsilyl)ethyl]oxy}methyl)oxy]-1-benzofuran-4-carboximidamide (3.45 g), 5-chloro-6-[(1-methylethyl)oxy]-3-pyridinecarboxylic acid (2.307 g), EDC (4.10 g), HOBT (0.328 g), Et₃N (3.73 mL) in THF (50 mL) was stirred at RT overnight. EtOAc (60 mL) was added and the organic solution was washed with water (20 mL). The organic fraction was dried over anhydrous magnesium sulfate. The dried solution was concentrated in vacuum to afford N-[({5-chloro-6-[(1-methylethyl)oxy]-3-pyridinyl}carbonyl)oxy]-7-[({[2-(trimethylsilyl)ethyl]oxy}methyl)oxy]-1-benzofuran-4-carboximidamide (D67) (5.5 g) as a yellow oil.

MS (ES): C₂₄H₃₀CIN₃O₆Si requires 519; found 520.2 (M+H⁺).

Description for D68

### 4-(5-{5-Chloro-6-[(1-methylethyl)oxy]-3-pyridinyl}-1,2,4-oxadiazol-3-yl)-1-benzofuran-7-ol (D68)

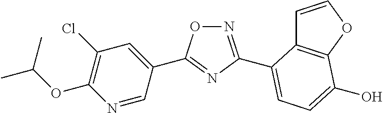

The mixture of N-[({5-chloro-6-[(1-methylethyl)oxy]-3-pyridinyl}carbonyl)oxy]-7-[({[2-(trimethylsilyl)ethyl]oxy}methyl)oxy]-1-benzofuran-4-carboximidamide (2.39 g), TBAF (6.01 g) in THF (20 mL) was stirred at 85° C. under nitrogen atmoshpere for 3 hours. EtOAc (60 mL) was added and the resulting solution was washed with water (2×15 mL). The organic fractions were combined. The combined solution was dried over anhydrous magnesium sulfate. The dried solution was concentrated. The resulting residue was purified by Teledyne ISCO (20% EtOAc in Petroleum) to afford 4-(5-{5-chloro-6-[(1-methylethyl)oxy]-3-pyridinyl}-1,2,4-oxadiazol-3-yl)-1-benzofuran-7-ol (D68) (730 mg) as a white solid.

MS (ES): C₁₈H₁₄CIN₃O₄ requires 371; found 372.1 (M+H⁺).

Description for D69

### Ethyl {(4-(5-{5-chloro-6-[(1-methylethyl)oxy]-3-pyridinyl} -1,2,4-oxadiazol-3-yl)-1-benzofuran-7-yl]oxy}acetate (D69)

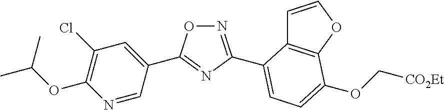

The mixture of ethyl bromoacetate (0.043 mL), 4-(5-{5-chloro-6-[(1-methylethyl)oxy]-3-pyridinyl}-1,2,4-oxadiazol-3-yl)-1-benzofuran-7-ol (120 mg), K₂CO₃ (134 mg) in THF (10 mL) was stirred at 50° C. overnight. EtOAc (30 mL) was added and the resulting mixture was washed with water (2×10 mL). The organic fractions were combined. The combined solution was dried over anhydrous magnesium sulfate. The dried solution was concentrated in vacuum to afford ethyl {[4-(5-{5-chloro-6-[(1-methylethyl)oxy]-3-pyridinyl}-1,2,4-oxadiazol-3-yl)-1-benzofuran-7-yl]oxy}acetate

(D69) (150 mg) as a yellow oil.

MS (ES): C₂₂H₂₀CIN₃O₆ requires 457; found 458.1 (M+H⁺).

Description for D70

### Ethyl 4-{[4-(5-{5-chloro-6-[(1-methylethyl)oxy]-3-pyridinyl}-1,2,4-oxadiazol-3-yl)-1-benzofuran-7-yl]oxy}butanoate (D70)

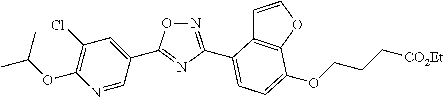

The mixture of 4-(5-{5-chloro-6-[(1-methylethyl)oxy]-3-pyridinyl}-1,2,4-oxadiazol-3-yl)-1-benzofuran-7-ol (100 mg), 4-(5-{5-chloro-6-[(1-methylethyl)oxy]-3-pyridinyl}-1,2,4-oxadiazol-3-yl)-1-benzofuran-7-ol (100 mg), K₂CO₃ (112 mg) in DMF (5 mL) was stirred at 60° C. for 5 hours. EtOAc (30 mL) was added and the resulting solution was washed with water (2×10 mL). The organic fractions were combined. The combined solution was dried over anhydrous magnesium sulfate. The dried solution was concentrated in vacuum to afford ethyl 4-{[4-(5-{5-chloro-6-[(1-methylethyl)oxy]-3-pyridinyl}-1,2,4-oxadiazol-3-yl)-1-benzofuran-7-yl]oxy}butanoate (D70) (130 mg) as a yellow oil.

MS (ES): C₂₄H₂₄CIN₃O₆ requires 485; found 486.2 (M+H⁺).

Description for D71

### 4-(5-{3-Chloro-4-[(1-methylethyl)oxy]phenyl}-1,2,4-oxadiazol-3-yl)-1-benzofuran-7-yl trifluoromethanesulfonate (D71)

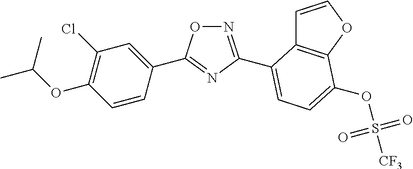

The mixture of 4-(5-{3-chloro-4-[(1-methylethyl)oxy]phenyl}-1,2,4-oxadiazol-3-yl)-1-benzofuran-7-ol (250 mg), 1,1,1-trifluoro-N-phenyl-N-[(trifluoromethyl)sulfonyl]methanesulfonamide (265 mg) and DIPEA (0.236 mL) in DCM (5 mL) was stirred at 25° C. under nitrogen atmoshpere overnight. DCM (15 mL) was added and the resulting mixture was washed with water (2×6 mL). The organic fractions were combined. The combined solution was dried over anhydrous magnesium sulfate. The dried solution was concentrated. The resulting residue was purified by Teledyne ISCO (10% EtOAc in Petroleum) to afford 4-(5-{3-chloro-4-[(1-methylethyl)oxy]phenyl}-1,2,4-oxadiazol-3-yl)-1-benzofuran-7-yl trifluoromethanesulfonate (D71) (295 mg) as a white solid.

MS (ES): C₂₀H₁₄CIF₃N₂O₆S requires 502; found 503.1 (M+H⁺).

Description for D72

### Ethyl 3-[4-(5-{3-chloro-4-[(1-methylethyl)oxy]phenyl}-1,2,4-oxadiazol-3-yl)-1-benzofuran-7-yl]propanoate (D72)

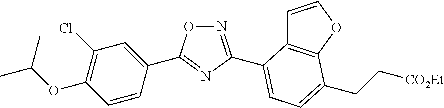

The mixture of 4-(5-{3-chloro-4-[(1-methylethyl)oxy]phenyl}-1,2,4-oxadiazol-3-yl)-1-benzofuran-7-yl trifluoromethanesulfonate (295 mg), tris(1,1-dimethylethyl)phosphane (17.0 mg), Pd₂(dba)₃ (26.9 mg), Cs₂CO₃ (573 mg) in DMF (2 mL) was stirred at RT under nitrogen atmoshpere, then bromo[3-(ethyloxy)-3-oxopropyl]zinc (289 mg) in THF (2 mL) was added quickly. The mixture was stirred at 115° C. under nitrogen atmoshpere overnight. EtOAc (30 mL) was added and the resulting solution was washed with water (2×10 mL). The organic fractions were combined. The combined solution was dried over anhydrous magnesium sulfate. The dried solution was concentrated. The resulting residue was purified by Teledyne ISCO (25% EtOAc in Petroleum) to afford ethyl 3-[4-(5-{3-chloro-4-[(1-methylenthyl)oxy]phenyl}-1,2,4-oxadiazol-3-yl)-1-benzofuran-7-yl]propanoate (D71) (120 mg) as a white solid.

MS (ES): C₂₄H₂₃CIN₂O₅ requires 454; found 455.2 (M+H⁺).

Description for D73

### 4-(5-{5-Chloro-6-[(1-methylethyl)oxy]-3-pyridinyl}-1,2,4-oxadiazol-3-yl)-1-benzofuran-7-yl trifluoromethanesulfonate (D73)

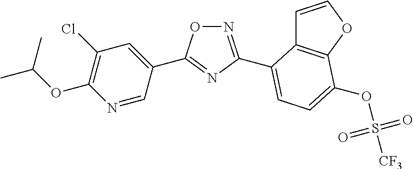

The mixture of 4-(5-{5-chloro-6-[(1-methylethyl)oxy]-3-pyridinyl}-1,2,4-oxadiazol-3-yl)-1-benzofuran-7-ol (600 mg), 1,1,1-trifluoro-N-phenyl-N-[(trifluoromethyl)sulfonyl]methanesulfonamide (634 mg), DIPEA (0.423 mL) in DCM (20 mL) was stirred at 25° C. overnight. DCM (20 mL) was added and the resulting mixture was washed with water (2×10 mL). The organic fractions were combined. The combined solution was dried over anhydrous magnesium sulfate. The dried solution was concentrated. The resulting residue was purified by Teledyne ISCO (10% EtOAc in Petroleum) to afford 4-(5-{5-chloro-6-[(1-methylenthyl)oxy]-3-pyridinyl}-1,2,4-oxadiazol-3-yl)-1-benzofuran-7-yl trifluoromethanesulfonate (D73) (600 mg) as a yellow solid.

MS (ES): C₁₉H₁₃CIF₃N₃O₆S requires 503; found 504.1 (M+H⁺).

Description for D74

### Ethyl 3-[4-(5-{5-chloro-6-[(1-methylethyl)oxy]-3-pyridinyl}-1,2,4-oxadiazol-3-yl)-1-benzofuran-7-yl]propanoate (D74)

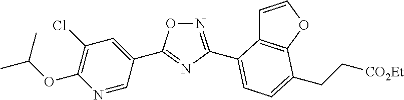

The mixture of 4-(5-{5-chloro-6-[(1-methylethyl)oxy]-3-pyridinyl}-1,2,4-oxadiazol-3-yl)-1-benzofuran-7-yl trifluoromethanesulfonate (300 mg), tris(1,1-dimethylethyl)phosphane (17 mg,), Pd₂(dba)₃ (27 mg), Cs₂CO₃ (582 mg) in DMF (2 mL) was stirred at RT under nitrogen atmoshpere, then bromo[3-(ethyloxy)-3-oxopropyl]zinc (293 mg) in THF (2 mL) was added quickly. The mixture was stirred at 115° C. under nitrogen atmoshpere overnight. EtOAc (30 mL) was added and the resulting solution was washed with water (2×10 mL). The organic fractions were combined. The combined solution was dried over anhydrous magnesium sulfate. The dried solution was concentrated. The resulting residue was purified by Teledyne ISCO (25% EtOAc in Petroleum) to afford ethyl 3-[4-(5-{5-chloro-6-[(1-methylethyl)oxy]-3-pyridinyl}-1,2,4-oxadiazol-3-yl)-1-benzofuran-7-yl]propanoate (D74) (200 mg) as a white solid.

MS (ES): C₂₃H₂₂CIN₃O₅ requires 455; found 456.2 (M+H⁺).

Description for (D75)

### Ethyl 3-{7-chloro-1-[(4-methylphenyl)sulfonyl]-1H-pyrrolo[2,3-c]pyridin-4-yl}propanoate (D75)

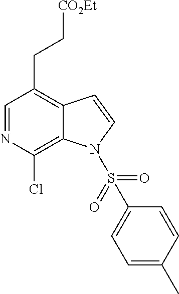

Under nitrogen atmosphere, the mixture of 4-bromo-7-chloro-1-[(4-methylphenyl)sulfonyl]-1H-pyrrolo[2,3-c]pyridine (226 mg), Pd₂(dba)₃ (54 mg), P(tBu)₃HBF₄ (17 mg), Cs₂CO₃ (38 mg) and bromo[3-(ethyloxy)-3-oxopropyl]zinc (433 mg) (0.5M in THF) in anhydrous THF (10 mL) was heated up to 80° C. (oil bath) for 1 hour. The reaction was monitored by LCMS. After the starting material was disappeared, the reaction mixture was cooled to RT, poured to water, extracted with EtOAc. The organic layer was washed with brine and dried over sodium sulphate, evaporated under reduced pressure. The residue was purified on ISCO using 40 g silica gel column eluting with 30% (EtOAC/Petroleum) to obtain 173 mg ethyl 3-{7-chloro-1-[(4-methylphenyl)sulfonyl]-1H-pyrrolo[2,3-c]pyridin-4-yl}propanoate (D75)

MS (ES): C₁₉H₁₉CIN₂O₄S requires 406.0; found 407.1 (M+H⁺).

Description for D76

### Ethyl 3-{7-cyano-1-[(4-methylphenyl)sulfonyl]-1H-pyrrolo[2,3-c]pyridin-4-yl}propanoate (D76)

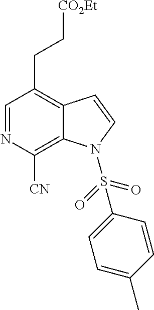

The mixture of ethyl 3-{7-chloro-1-[(4-methylphenyl)sulfonyl]-1H-pyrrolo[2,3-c]pyridin-4-yl}propanoate (220 mg) and copper(I) cyanide (97 mg), copper(I) iodide (31 mg) in DMF (3 mL) was heated at 180° C. in the microwave for one hour. LCMS showed the reaction worked well. The mixture was poured into 20% NH4OH, extracted with EtOAc (50 mL×3). The combined organic layer was washed with brine (50 mL), dried over sodium sulphate, evaporated in vacuo. The residue was purified by ISCO on silica gel column (12 g) eluting with 10% EA/PE to obtain 120 mg ethyl 3-{7-cyano-1-[(4-methylphenyl)sulfonyl]-1H-pyrrolo[2,3-c]pyridin-4-yl}propanoate (D76).

MS (ES): C₂₀H₁₉N₃O₄S requires 397.1; found 398.2 (M+H⁺).

Description for D77

### Ethyl 3-{7-[(Z)-(hydroxyamino)(imino)methyl]-1-[(4-methylphenyl)sulfonyl]-1H-pyrrolo[2,3-c]pyridin-4-yl}propanoate (D77)

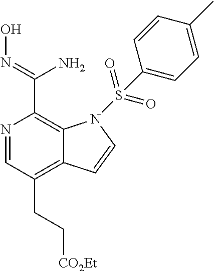

The mixture of ethyl 3-{7-cyano-1-[(4-methylphenyl)sulfonyl]-1H-pyrrolo[2,3-c]pyridin-4-yl}propanoate (120 mg), hydroxylamine hydrochloride (126 mg), molecular sieves (300 mg) and sodium bicarbonate (152 mg) in ethanol (10 mL) was heated up to reflux and stirred for 3 hours. Two pot additional hydroxylamine hydrochloride (98 mg) and sodium bicarbonate (177 mg) was added. The reaction was monitored by LCMS. The reaction mixture was cooled to RT and the solid was removed by filtration and washed successfully waith ethanol and THF. The filtration was evaporated under reduced pressure to obtain the crude product, it was purified by ISCO on 12 gram silica gel eluting with 60% EA/PE to obtain 85 mg ethyl 3-{7-[(Z)-(hydroxyamino)(imino)methyl]-1-[(4-methylphenyl)sulfonyl]-1H-pyrrolo[2,3-c]pyridin-4-yl}propanoate (D77).

MS (ES): C₂₀H₂₂N₄O₅S requires 430.1; found 431.2 (M⁺).

Description for D78

### Ethyl 3-[7-(5-{3-chloro-4-[(1-methylethyl)oxy]phenyl}-1,2,4-oxadiazol-3-yl)-1H-pyrrolo[2,3-c]pyridin-4-yl]propanoate (D78)

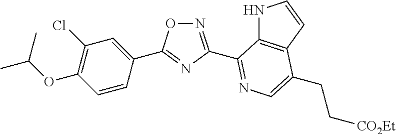

Under nitrogen atmosphere, the mixture of 3-chloro-4-[(1-methylethyl)oxy]benzoic acid (47 mg), EDC (57 mg), HOBT (45 mg) in THF (2 mL) was stirred at RT for 1 hour, then the solution of ethyl 3-{7-[(Z)-(hydroxyamino)(imino)methyl]-1-[(4-methylphenyl)sulfonyl]-1H-pyrrolo[2,3-c]pyridin-4-yl}propanoate (85 mg) in THF (1 mL) was added. The mixture was stirred at RT for 1 hour. LCMS showed no starting material left. TBAF (258 mg) was added and the mixture was heated up to 120° C. on a biotage microwave stirring one hour. LCMS showed no starting material left. After cooled to RT, the mixture was partinationed to water and ethyl acetate. The organic layer was dried over sodium sulphate, evaporated under reduced pressure and the residue was purified by ISCO on silica gel eluting with methylene chloride to obtain 35 mg ethyl 3-[7-(5-{3-chloro-4-[(1-methylethyl)oxy]phenyl}-1,2,4-oxadiazol-3-yl)-1H-pyrrolo[2,3-c]pyridin-4-yl]propanoate (D78).

MS (ES): C₂₃H₂₃CIN₄O₄ requires 454.1; found 455.2 (M+H⁺).

Description for D79

### Ethyl 3-{7-[{[({3-cyano-4-[(1-methylethyl)oxy]phenyl}carbonyl)oxy]amino}(imino)methyl]-1-[(4-methylphenyl)sulfonyl]-1H-pyrrolo[2,3-c]pyridin-4-yl}propanoate (D79)

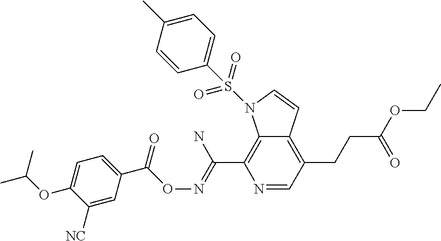

The mixture of ethyl 3-{7-[(hydroxyamino)(imino)methyl]-1-[(4-methylphenyl)sulfonyl]-1H-pyrrolo[2,3-c]pyridin-4-yl}propanoate (70 mg), 3-cyano-4-[(1-methylethyl)oxy]benzoic acid (40 mg), HOBT (5 mg), EDC (62 mg), Et₃N (0.057 mL) in THF (10 mL) was stirred at RT for 2 hours. LCMS showed reaction was complete. Partitioned the mixture with EA and water, the organic layer was concentrated in vacuum to give crude product which was used in the next step without further purification.

Description for D80

### Ethyl 3-(7-(5-{3-cyano-4-[(1-methylethyl)oxy]phenyl}-1,2,4-oxadiazol-3-yl)-1H-pyrrolo[2,3-c]pyridin-4-yl]propanoate (D80)

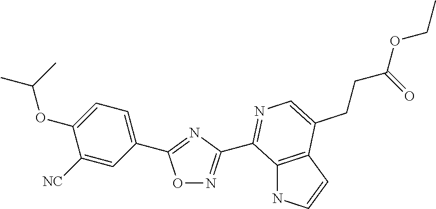

The mixture of ethyl 3-{7-[{[({3-cyano-4-[(1-methylethyl)oxy]phenyl}carbonyl)oxy]amino}(imino)methyl]-1-[(4-methylphenyl)sulfonyl]-1H-pyrrolo[2,3-c]pyridin-4-yl)propanoate (100 mg) and TBAF (212 mg) in THF (5 mL) was stirred at 85° C. for 2 hours. LCMS showed reaction completed. Partitioned the mixture with EA and water, the organic layer was concentrated in vacuum and purified by ISCO (PE/EA, 3/1) to afford 30 mg ethyl 3-[7-(5-{3-cyano-4-[(1-methylethyl)oxy]phenyl}-1,2,4-oxadiazol-3-yl)-1H-pyrrolo[2,3-c]pyridin-4-yl]propanoate (D80).

MS (ES): C₂₄H₂₃N₅O₄ requires 445.1; found 446.2 (M+H⁺).

### EXAMPLE 1

### 3-[7-(5-{3-Chloro-4-[(1-methylethyl)oxy]phenyl}-1,2,4-oxadiazol-3-yl)-1H-indol-4-yl]propanoic acid (El)

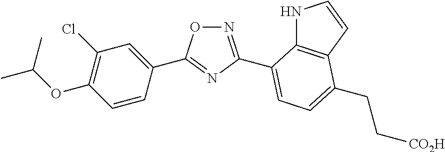

A mixture of ethyl 3-[7-(5-{3-chloro-4-[(1-methylethyl)oxy]phenyl}-1,2,4-oxadiazol-3-yl)-1H-indol-4-yl]propanoate (58 mg), sodium hydroxide (50 mg) (1 mL, 5% solution in water) and ethanol (10 mL) was heated up to 70° C. for one hour. After cooled to rt, the mixture was neutralized to pH=6-7 with 1N HCl. The mixture was evaporated under reduced pressure and extracted with ethyl acetate, the organic layer was washed with brine, dried over sodium sulphate, evaporated under reduced pressure and the residue was purified by MDAP obtaining 20 mg 3-[7-(5-{3-Chloro-4-[(1-methylethyl)oxy]phenyl}-1,2,4-oxadiazol-3-yl)-1H-indol-4-yl]propanoic acid (E1).

δH (CDCl₃, 400 MHz): 1.45 (6H, d), 2.86 (2H, t), 3.34 (2H, t), 4.71 (1H, m), 6.69 (1H, t), 7.09 (2H, m), 7.38 (1H, t), 8.10-8.04 (2H, m), 8.27 (1H, d), 9.88 (1H, s, br). MS (ES): C₂₂H₂₀CIN₃O₄ requires 425.1; found 426.1 (M+H⁺).

### EXAMPLE 2

### 3-[7-(5-{3-Chloro-4-[(1-methylethyl)oxy]phenyl}-1,2,4-oxadiazol-3-yl)-1-methyl-1H-indol-4-yl]propanoic acid (E2)

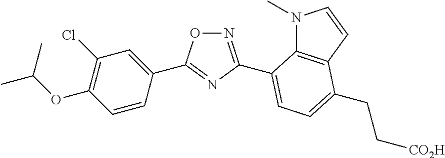

The mixture of ethyl 3-[7-(5-{3-chloro-4-[(1-methylethyl)oxy]phenyl}-1,2,4-oxadiazol-3-yl)-1H-indol-4-yl]propanoate (55 mg), dimethyl carbonate (218 mg) and DABCO (13 mg) in DMF (3 mL) was heated up to 120° C. for 5 hours. After removing the solvent and excess dimethyl carbonate, the residue was dissolved in ethyl acetate and washed with water and brine. The solvent was evaporated to obtain 56 mg 3-[7-(5-{3-chloro-4-[(1-methylethyl)oxy]phenyl}-1,2,4-oxadiazol-3-yl)-1-methyl-1H-indol-4-yl]propanoate. A mixture of ethyl 3-[7-(5-{3-chloro-4-[(1-methylethyl)oxy]phenyl}-1,2,4-oxadiazol-3-yl)-1-methyl-1H-indol-4-yl]propanoate (56 mg), sodium hydroxide (145 mg) (3 mL, 5% solution in water) and THF(10 mL) was heated up to 70° C. for one hour. After cooled to rt, the mixture was neutralized to pH=6-7 with 1N HCl. The mixture was evaporated under reduced pressure and extracted with ethyl acetate, the organic layer was washed with brine, dried over sodium sulphate, evaporated under reduced pressure and the residue was purified by MDAP obtaining 45 mg of 3-[7-(5-{3-chloro-4-[(1-methylethyl)oxy]phenyl}-1,2,4-oxadiazol-3-yl)-1-methyl-1H-indol-4-yl]propanoic acid (E2).

δH (CDCl₃, 400 MHz): 1.45 (6H, d), 2.83 (2H, t), 3.30 (2H, t), 3.76 (3H, s), 4.71 (1H, m), 6.61 (1H, d), 7.08-7.04 (3H, m), 7.49 (1H, d), 8.06 (1H, m), 8.24 (1H, d). MS (ES): C₂₃H₂₂CIN₃O₄ requires 439.2; found 440.1 (M+H⁺).

### EXAMPLE 3

### 3-[7-(5-{3-Chloro-4-[(1-methylethyl)oxy]phenyl}-1,2,4-oxadiazol-3-yl)-1-ethyl-1H-indol-4-yl]propanoic acid (E3)

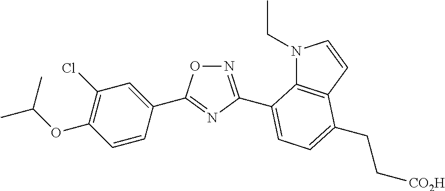

At RT, under nitrogen, potassium hydroxide (30 mg) was added to the solution of ethyl 3-[7-(5-{3-chloro-4-[(1-methylethyl)oxy]phenyl}-1,2,4-oxadiazol-3-yl)-1H-indol-4-yl]propanoate (50 mg) and bromoethane (120 mg) in dimethyl sulfoxide (4 mL). The resulting mixture was stirred at RT for 3 hours. LCMS showed the alkylation of the nitrogen of indole cycle was completed. To this mixture was added 6 mL water and 5 mL ethanol, the resulting mixture was heated up to 65° C. stirring for 30 min. The reaction was monitored by LCMS showing the hydrolysis was completed. After neutralized the reaction mixture to pH 5-6 with concentrated HCl at 0° C., solvent was removed under reduced pressure. The residue was extracted with ethyl acetate and the combined organic layer was washed with brine, dried over sodium sulphate and evaporated to dryness. The crude product was purified by MDAP to obtain 30 mg of compound 3-[7-(5-{3-chloro-4-[(1-methylethyl)oxy]phenyl}-1,2,4-oxadiazol-3-yl)-1-ethyl-1H-indol-4-yl]propanoic acid (E3).

δH (CDCl₃, 400 MHz): 1.18 (3H, t), 1.44 (6H, d), 2.84 (2H, t), 3.31 (2H, t), 4.27 (2H, q), 4.71 (1H, m), 6.61 (1H, d), 7.05 (2H, m), 7.17 (1H, d), 7.49 (1H, d), 8.07 (1H, d), 8.25 (1H, d). MS (ES): C₂₄H₂₄CIN₃O₄ requires 453.1; found 454.2 (M+H⁺).

### EXAMPLE 4

### 3-[7-(5-{5-Chloro-6-[(1-methylethyl)oxy]-3-pyridinyl}-1,2,4-oxadiazol-3-yl)-1H-indol-4-yl]propanoic acid (E4)

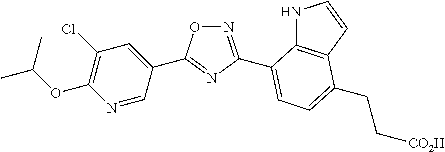

The mixture of ethyl 3-[7-(5-{5-chloro-6-[(1-methylethyl)oxy]-3-pyridinyl}-1,2,4-oxadiazol-3-yl)-1H-indol-4-yl]propanoate (30 mg), NaOH (26 mg) in ethanol (2 mL) and water (5 mL) was stirred at 70° C. for 2 hours. The organics was removed under vacuum and the resulting solution was acidified with aqueous HCl (1 mL). The resulting mixture was extracted with EtOAc The organic fractions were combined and concentrated in vacuum. The resulting residue was purified by MDAP to afford 3-[7-(5-{5-chloro-6-[(1-methylethyl)oxy]-3-pyridinyl}-1,2,4-oxadiazol-3-yl)-1H-indol-4-yl]propanoic acid (E4) (10 mg) as white solid.

δH (CDCl₃, 400 MHz): 1.46 (6H, d), 2.88 (2H, t), 3.36 (2H, t), 5.52 (1H, m), 6.72 (1H, t), 7.15 (1H, d), 7.41 (1H, t), 8.06 (1H, d), 8.44 (1H, s), 8.93 (1H, s), 9.87 (1H, s). MS (ES): C₂₁H₁₉CIN₄O₄ requires 426; found 427.1 (M+H⁺).

### EXAMPLE 5

### 3-[7-(5-{5-Chloro-6-[(1-methylethyl)oxy]-3-pyridinyl}-1,2,4-oxadiazol-3-yl)-1-methyl-1H-indol-4-yl]propanoic acid (E5)

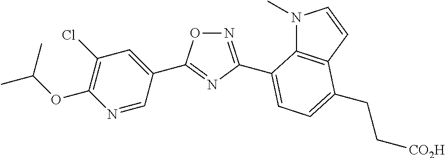

The mixture of ethyl 3-(7-(5-{5-chloro-6-[(1-methylethyl)oxy]-3-pyridinyl}-1,2,4-oxadiazol-3-yl)-1-methyl-1H-indol-4-yl]propanoate (100 mg), NaOH (9 mg) in ethanol (3 mL) and water (3 mL) was stirred at 70° C. for 2 hours. The organics was removed under vacuum and the resulting solution was acidified with aqueous HCl (1 mL). The resulting mixture was extracted with EtOAc The organic fractions were combined and concentrated in vacuum. The resulting residue was purified by MDAP to afford 3-[7-(5-{5-chloro-6-[(1-methylethyl)oxy]-3-pyridinyl}-1,2,4-oxadiazol-3-yl)-1-methyl-1H-indol-4-yl]propanoic acid (E5) (40 mg) as white solid.

δH (DMSO-d₆, 400 MHz): 1.38 (6H, d), 2.64 (2H, t), 3.15 (2H, t), 3.68 (3H, s), 5.45 (1H, m), 6.65 (1H, s), 7.02 (1H, d), 7.38 (2H, m), 8.56 (1H, s), 8.94 (1H, s). MS (ES): C₂₂H₂₁CIN₄O₄ requires 440; found 441.2 (M+H+).

### EXAMPLE 6

### 3-[7-(5-{3-Cyano-4-[(1-methylethyl)oxy]phenyl}-1,2,4-oxadiazol-3-yl)-1H-indol-4-yl]propanoic acid (E6)

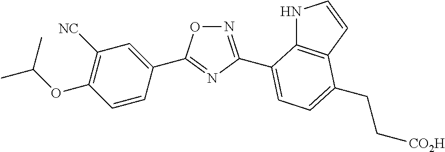

The mixture of ethyl 3-[7-(5-{3-cyano-4-[(1-methylethyl)oxy]phenyl}-1,2,4-oxadiazol-3-yl)-1H-indol-4-yl]propanoate (200 mg), NaOH (180 mg) in THF (2 mL), ethanol (2 mL) and water (2 mL) was stirred at 70° C. for 2 hours. The organics was removed under vacuum and the resulting solution was acidified with aqueous HCl (2 mL). The resulting mixture was extracted with EtOAc The organic fractions were combined and concentrated in vacuum. The resulting residue was purified by MDAP to afford 3-[7-(5-{3-cyano-4-[(1-methylethyl)oxy]phenyl}-1,2,4-oxadiazol-3-yl)-1H-indol-4-yl]propanoic acid (E6) (107 mg) as white solid.

δH (DMSO-d₆, 400 MHz): 1.39 (6H, d), 2.66 (2H, t), 3.17 (2H, t), 5.00 (1H, m), 6.68 (1H, s), 7.06 (1H, d), 7.47 (1H, s), 7.56 (1H, d), 7.86 (1H, d), 8.50(1H, d), 8.74(1H, s), 11.14(1H, s), 12.18 (1H, s). MS (ES): C₂₃H₂₀N₄O₄ requires 416; found 417.2 (M+H⁺).

### EXAMPLE 7

### 3-[7-(5-{3-Cyano-4-[(1-methylethyl)oxy]phenyl}-1,2,4-oxadiazol-3-yl)-1-methyl-1H-indol-4-yl]propanoic acid (E7)

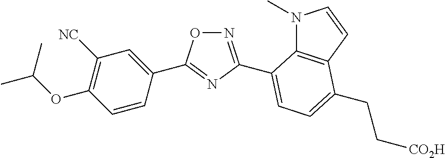

The mixture of ethyl 3-[7-(5-{3-cyano-4-[(1-methylethyl)oxy]phenyl}-1,2,4-oxadiazol-3-yl)-1-methyl-1H-indol-4-yl]propanoate (150 mg), NaOH (13 mg) in THF (2 mL), methanol (2 mL) and water (2 mL) was stirred at 70° C. for one hour. The organics was removed under vacuum and the resulting solution was acidified with aqueous HCl (2 mL). The resulting mixture was extracted with EtOAc The organic fractions were combined and concentrated in vacuum. The resulting residue was purified by MDAP to afford 3-[7-(5-{3-cyano-4-[(1-methylethyl)oxy]phenyl}-1,2,4-oxadiazol-3-yl)-1-methyl-1H-indol-4-yl]propanoic acid (E7) (100 mg) as white solid.

δH (DMSO-d₆, 400 MHz): 1.37 (6H, d), 2.64 (2H, t), 3.15 (2H, t), 3.68 (3H, s), 4.97 (1H, m), 6.65 (1H, s), 7.03 (1H, d), 7.40 (2H, m), 7.54 (1H, d), 8.40 (1H, d), 8.52(1H, s), 12.18 (1H, s). MS (ES): C₂₄H₂₂N₄O₄ requires 430; found 431.2 (M+H⁺).

### EXAMPLE 8

### 4-[7-(5-{3-Chloro-4-[(1-methylethyl)oxy]phenyl}-1,2,4-oxadiazol-3-yl)-1H-indol-4-yl]butanoic acid (E8)

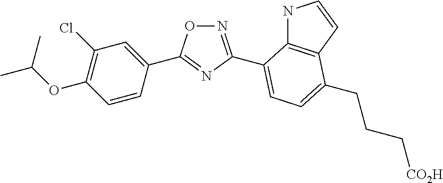

The mixture of ethyl 4-[7-(5-{3-chloro-4-[(1-methylethyl)oxy]phenyl}-1,2,4-oxadiazol-3-yl)-1H-indol-4-yl]butanoate (80 mg), NaOH (26 mg) in THF (1 mL) and water (5 mL) was stirred at 70° C. for 3 hours. The organics was removed under vacuum and the resulting solution was acidified with aqueous HCl (1 mL). The resulting mixture was extracted with EtOAc The organic fractions were combined and concentrated in vacuum. The resulting residue was purified by MDAP to afford 4-[7-(5-{3-chloro-4-[(1-methylethyl)oxy]phenyl}-1,2,4-oxadiazol-3-yl)-1H-indol-4-yl]butanoic acid (E8) (50 mg) as white solid.

δH (CDCl₃, 400 MHz): 1.58 (6H, d), 2.16 (2H, m), 2.46 (2H, t), 3.07 (2H, t), 4.74 (1H, m), 6.71 (1H, s), 7.10 (2H, m), 7.40 (1H, s), 8.10 (1H, d), 8.13 (1H, d), 8.30 (1H, s), 9.88 (1H, s). MS (ES): C₂₃H₂₂CIN₃O₄ requires 439; found 440.2 (M+H⁺).

### EXAMPLE 9

### 4-[7-(5-{3-Chloro-4-[(1-methylethyl)oxy]phenyl}-1,2,4-oxadiazol-3-yl)-1-methyl-1H-indol-4-yl]butanoic acid (E9)

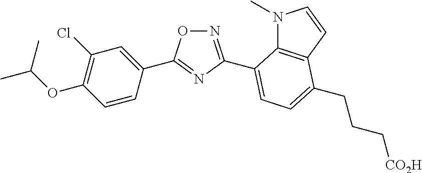

The mixture of ethyl 4-[7-(5-{3-chloro-4-[(1-methylethyl)oxy]phenyl}-1,2,4-oxadiazol-3-yl)-1-methyl-1H-indol-4-yl]butanoate (113 mg), NaOH (94 mg) in ethanol (1 mL) and water (4 mL) was stirred at 70° C. for 2 hours. The organics was removed under vacuum and the resulting solution was acidified with aqueous HCl (1 mL). The resulting mixture was extracted with EtOAc The organic fractions were combined and concentrated in vacuum. The resulting residue was purified by MDAP to afford 4-[7-(5-{3-chloro-4-[(1-methylethyl)oxy]phenyl}-1,2,4-oxadiazol-3-yl)-1-methyl-1H-indol-4-yl]butanoic acid (E9) (60 mg) as white solid.

δH (CDCl₃, 400 MHz): 1.46 (6H, d), 2.13 (2H, m), 2.44 (2H, t), 3.03 (2H, t), 3.78 (3H, s), 4.73 (1H, m), 6.63 (1H, d), 7.07 (3H, m), 7.49 (1H, d), 8.09 (1H, d), 8.26 (1H, s). MS (ES): C₂₄H₂₄CIN₃O₄ requires 453; found 454.2 (M+H⁺).

### EXAMPLE 10

### 4-[7-(5-{5-Chloro-6.-[(1-methylethyl)oxy]-3-pyridinyl}-1,2,4-oxadiazol-3-yl)-1H-indol-4-yl]butanoic acid (E10)

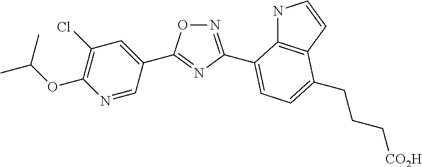

The mixture of ethyl 4-[7-(5-{5-chloro-6-[(1-methylethyl)oxy]-3-pyridinyl}-1,2,4-oxadiazol-3-yl)-1H-indol-4-yl]butanoate (60 mg), NaOH (5 mg) in ethanol (3 mL) and water (3 mL) was stirred at 70° C. for 2 hours. The organics was removed under vacuum and the resulting solution was acidified with aqueous HCl (1 mL). The resulting mixture was extracted with EtOAc The organic fractions were combined and concentrated in vacuum. The resulting residue was purified by MDAP to afford 4-[7-(5-{5-chloro-6-[(1-methylethyl)oxy]-3-pyridinyl}-1,2,4-oxadiazol-3-yl)-1H-indol-4-yl]butanoic acid (E10) (51 mg) as white solid.

δH (DMSO-d₆, 400 MHz): 1.39 (6H, d), 1.93 (2H, m), 2.29 (2H, t), 2.94 (2H, t), 5.46 (1H, m), 6.68 (1H, t), 7.03 (1H, d), 7.46 (1H, t), 7.87 (1H, d), 8.75 (1H, d), 9.05 (1H, s), 11.09(1H, s). MS (ES): C₂₂H₂₁CIN₄O₄requires 440; found 441.2 (M+H⁺).

### EXAMPLE 11

### 4-[7-(5-{5-Chloro-6-[(1-methylethyl)oxy]-3-pyridinyl}-1,2,4-oxadiazol-3-yl)-1-methyl-1H-indol-4-yl]butanoic acid (E11)

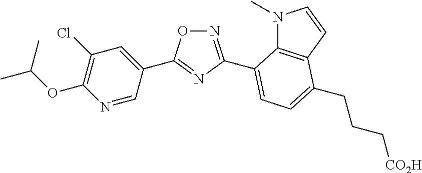

The mixture of ethyl 4-[7-(5-{5-chloro-6-[(1-methylethyl)oxy]-3-pyridinyl}-1,2,4-oxadiazol-3-yl)-1-methyl-1H-indol-4-yl]butanoate (85 mg), NaOH (7 mg) in ethanol (3 mL) and water (3 mL) was stirred at 70° C. for 0.5 hour. The organics was removed under vacuum and the resulting solution was acidified with aqueous HCl (1 mL). The resulting mixture was extracted with EtOAc The organic fractions were combined and concentrated in vacuum. The resulting residue was purified by MDAP to afford 4-[7-(5-{5-chloro-6-[(1-methylethyl)oxy]-3-pyridinyl}-1,2,4-oxadiazol-3-yl)-1-methyl-1H-indol-4-yl]butanoic acid (E11) (18 mg) as white solid.

δH (CDCl₃, 400 MHz): 1.46 (6H, d), 2.15 (2H, m), 2.45 (2H, t), 3.04 (2H, t), 3.80 (3H, s), 5.53 (1H, m), 6.65 (1H, s), 7.05 (2H, m), 7.50 (1H, d), 8.40 (1H, s), 8.90 (1H, s). MS (ES): C₂₃H₂₃CIN₄O₄ requires 454; found 455.2 (M+H⁺).

### EXAMPLE 12

### 3-[4-(5-{3-Chloro-4-[(1-methylethyl)oxy]phenyl}-1,2,4-oxadiazol-3-yl)-1H-indol-7-yl]propanoic acid (E12)

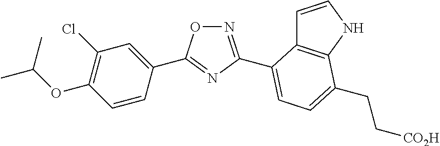

The mixture of ethyl 3-[4-(5-{3-chloro-4-[(1-methylethyl)oxy]phenyl}-1,2,4-oxadiazol-3-yl)-1H-indol-7-yl]propanoate (65 mg)and sodium hydroxide (50 mg) (20% solution, 0.25 mL) in ethanol (10 mL) and THF (1 mL) was heated up to 60° C. for 30 min. The reaction was monitored by LCMS. After cooled to RT, the mixture was neutralized to pH=6-7 by 1N HCl. The solvent was removed under reduced pressure, to the residue was added water and extracted by ethyl acetate. After removed the solvent, the residue was purified by MDAP to obtain the compound 3-[4-(5-{3-chloro-4-[(1-methylethyl)oxy]phenyl}-1,2,4-oxadiazol-3-yl)-1H-indol-7-yl]propanoic acid (E12).

δH (DMSO-d₆, 400 MHz): 1.35 (6H, d), 2.68 (2H, t), 3.18 (2H, t), 4.87 (1H, m), 7.08-7.13 (2H, m), 7.44 (1H, d), 7.53 (1H, d), 7.84 (1H, d), 8.13 (1H, m), 8.20 (1H, d), 11.5 (1H, s, br), 12.1 (1H, s, br). MS (ES): C₂₂H₂₀C1N₃O₄ requires 425.1; found 426.1 (M+H⁺).

### EXAMPLE 13

### 3-(4-(5-{3-Chloro-4-[(1-methylethyl)oxy]phenyl}-1,2,4-oxadiazol-3-yl)-1-methyl-1H-indol-7-yl]propanoic acid (E13)

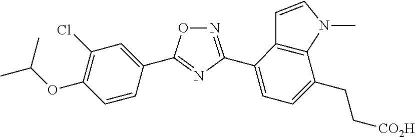

To the suspension of ethyl 3-[4-(5-{3-chloro-4-[(1-methylethyl)oxy]phenyl}-1,2,4-oxadiazol-3-yl)-1-methyl-1H-indol-7-yl]propanoate (65 mg) in ethanol (8 mL) was added 0.2 mL 20% sodium hydroxide (40 mg). The resulting mixture was heated up to 60° C. for 1 hour. The reaction was monitored by LCMS. After cooled to RT, the mixture was neutralized to pH=6-7 by 1N HCl. The solvent was removed under reduced pressure, to the residue was added water and extracted by ethyl acetate. After removed the solvent, the residue was purified by MDAP to obtain 43 mg 3-[4-(5-{3-chloro-4-[(1-methylethyl)oxy]phenyl}-1,2,4-oxadiazol-3-yl)-1-methyl-1H-indol-7-yl]propanoic acid (E13).

δH (DMSO-d₆), 400 MHz): 1.36 (6H, d), 2.67 (2H, t), 3.41 (2H, t), 4.10 (3H, s), 4.88 (1H, m), 7.07-7.12 (2H, m), 7.45 (2H, m), 7.83 (1H, d), 8.11-8.19 (1H, m), 8.20 (1H, d), 12.28 (1H, s, br). MS (ES): C₂₃H₂₂CIN₃O₄ requires 439.2; found 440.1 (M+H⁺).

### EXAMPLE 14

### 3-[4-(5-{3-Chloro-4-[(1-methylethyl)oxy]phenyl}-1,2,4-oxadiazol-3-yl)-1-ethyl-1H-indol-7-yl]propanoic acid (E14)

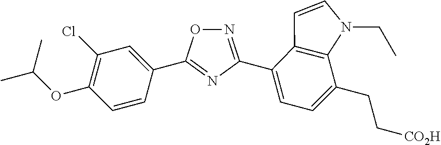

To the suspension of ethyl 3-[4-(5-{3-chloro-4-[(1-methylethyl)oxy]phenyl}-1,2,4-oxadiazol-3-yl)-1-ethyl-1H-indol-7-yl]propanoate (93 mg) in was added 0.2 mL 20% sodium hydroxide (40 mg). The resulting mixture was heated up to 60° C. for 1 hour. The reaction was monitored by LCMS. After cooled to RT, the mixture was neutralized to pH=6-7 by 1N HCl. The solvent was removed under reduced pressure, to the residue was added water and extracted by ethyl acetate. After removed the solvent, the residue was purified by MDAP to obtain 74 mg 3-[4-(5-{3-chloro-4-[(1-methylethyl)oxy]phenyl}-1,2,4-oxadiazol-3-yl)-1-ethyl-1H-indol-7-yl]propanoic acid (E14).

δH (CDCl₃+1 drop CD₃OD, 400 MHz): 1.45-1.49 (9H, m), 2.75 (2H, t), 3.40-3.44 (2H, t), 4.42 (2H, q), 4.72 (1H, m), 7.07 (1H, d), 7.13 (1H, d), 7.24 (1H, d), 7.32 (1H, d), 7.94 (1H, d), 8.10 (1H, d), 8.28 (1H, d). MS (ES): C₂₄H₂₄CIN₃O₄ requires 453.1; found 454.2 (M+H⁺).

### EXAMPLE 15

### 4-[4-(5-{3-Chloro-4-[(1-methylethyl)oxy]phenyl}-1,2,4-oxadiazol-3-yl)-1H-indol-7-yl]butanoic acid (E15)

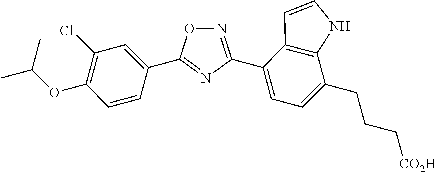

The mixture of ethyl 4-[4-(5-{3-chloro-4-[(1-methylethyl)oxy]phenyl}-1,2,4-oxadiazol-3-yl)-1H-indol-7-yl]butanoate (65 mg)and sodium hydroxide (56 mg) (20% solution, 0.25 mL) in ethanol (10 mL) and THF (1 mL) was heated up to 60° C. for 30 min. The reaction was monitored by LCMS. After cooled to RT, the mixture was neutralized to pH=6-7 by 1N HCl. The solvent was removed under reduced pressure, to the residue was added water and extracted by ethyl acetate. After removed the solvent, the residue was purified by MDAP to obtain 25 mg 4-[4-(5-{3-chloro-4-[(1-methylethyl)oxy]phenyl}-1,2,4-oxadiazol-3-yl)-1H-indol-7-yl]butanoic acid.

δH (DMSO-d₆, 400 MHz): 1.36 (6H, d), 1.93 (2H, m), 2.30 (2H, t), 2.95 (2H, t), 4.88 (1H, m), 7.09 (2H, m), 7.44 (1H, d), 7.54 (1H, d), 7.85 (1H, d), 8.13 (1H, d), 8.20 (1H, d), 11.48 (1H, s, br), 12.04 (1H, s, br). MS (ES): C₂₃H₂₂CIN₃O₄ requires 439.1; found 440.2 (M+H⁺).

### EXAMPLE 16

### 4-(4-(5-{3-Chloro-4-[(1-methylethyl)oxy]phenyl}-1,2,4-oxadiazol-3-0)-1-methyl-1H-indol-7-yl]butanoic acid (E16)

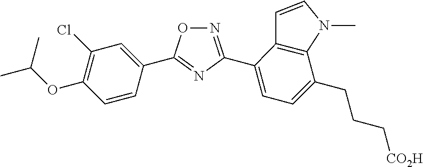

The mixture of ethyl 4-[4-(5-{3-chloro-4-[(1-methylethyl)oxy]phenyl}-1,2,4-oxadiazol-3-yl)-1-methyl-1H-indol-7-yl]butanoate (100 mg) and sodium hydroxide (50 mg) (20% solution, 0.25 mL) in ethanol and THF was heated up to reflux for 30 min. The reaction was monitored by LCMS. After cooled to RT, the mixture was neutralized to pH=6-7 by 1N HCl. The solvent was removed under reduced pressure, to the residue was added water and extracted by ethyl acetate. After removed the solvent, the residue was purified by MDAP to obtain 65 mg 4-[4-(5-{3-chloro-4-[(1-methylethyl)oxy]phenyl}-1,2,4-oxadiazol-3-yl)-1-methyl-1H-indol-7-yl]butanoic acid (E16).

δH (DMSO-d₆, 400 MHz): 1.36 (6H, d), 1.91 (2H, m), 2.37 (2H, t), 3.14 (2H, t), 4.08 (3H, s), 4.88 (1H, m), 7.08 (2H, m), 7.45 (2H, m), 7.84 (1H, d), 8.12 (1H, d), 8.20 (1H, d), 12.14 (1H, s, br). MS (ES): C₂₄H₂₄CIN₃O₄ requires 453.1; found 454.2 (M+H⁺).

### EXAMPLE 17

### 4-[4-(5-{3-Chloro-4-[(1-methylethyl)oxy]phenyl}-1,2,4-oxadiazol-3-yl)-1-ethyl-1H-indol-7-yl]butanoic acid (E17)

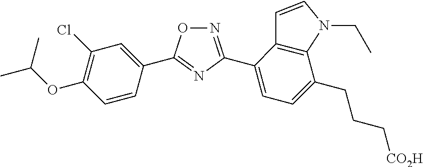

To the suspension of ethyl 4-[4-(5-{3-chloro-4-[(1-methylethyl)oxy]phenyl}-1,2,4-oxadiazol-3-yl)-1-ethyl-1H-indol-7-yl]butanoate (75 mg) in ethanol (8 mL) was added 0.2 mL 20% sodium hydroxide (40 mg). The resulting mixture was heated up to 60° C. for 1 hour. The reaction was monitored by LCMS. After cooled to RT, the mixture was neutralized to pH=6-7 by 1N HCl. The solvent was removed under reduced pressure, to the residue was added water and extracted by ethyl acetate. After removed the solvent, the residue was purified by MDAP to obtain 50 mg 4-[4-(5-{3-chloro-4-[(1-methylethyl)oxy]phenyl}-1,2,4-oxadiazol-3-yl)-1-ethyl-1H-indol-7-yl]butanoic acid (E17).

δH (CDCl₃+1drop CD₃OD, 400 MHz): 1.45 (9H, m), 2.08 (2H, m), 2.51 (2H,m), 3.13 (2H, t), 4.41 (2H, q), 4.70 (1H, m), 7.08 (2H, m), 7.22 (1H, d), 7.34 (1H, d), 7.95 (1H, d), 8.08 (1H, dd), 8.28 (1H, d). MS (ES): C₂₆H₂₆CIN₃O₄ requires 467.1; found 468.2 (M+H⁺).

### EXAMPLE 18

### 3-[4-(5-{5-Chloro-6-[(1-methylethyl)oxy]-3-pyridinyl}-1,2,4-oxadiazol-3-yl)-1H-indol-7-yl]propanoic acid (E18)

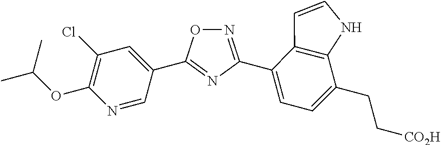

The mixture of ethyl 3-[4-(5-{5-chloro-6-[(1-methylethyl)oxy]-3-pyridinyl}-1,2,4-oxadiazol-3-yl)-1H-indol-7-yl]propanoate (50 mg) and sodium hydroxide (50 mg) (20% solution, 0.25 mL) in ethanol (10 mL) and THF (1 mL) was heated up to 60° C. for 30 min. The reaction was monitored by LCMS. After cooled to RT, the mixture was neutralized to pH=6-7 by 1N HCl. The solvent was removed under reduced pressure, to the residue was added water and extracted by ethyl acetate. After removed the solvent, the residue was purified by MDAP to obtain 15 mg 3-[4-(5-{5-chloro-6-[(1-methylethyl)oxy]-3-pyridinyl}-1,2,4-oxadiazol-3-yl)-1H-indol-7-yl]propanoic acid (E18).

δH (DMSO-d₆, 400 MHz): 1.38 (6H, d), 2.68 (2H, t), 3.18 (2H, t), 5.45 (1H, m), 7.12 (2H, m), 7.53 (1H, s), 7.84 (1H, d), 8.55 (1H, s), 8.93 (1H, d), 11.55 (1H, s, br), 12.16 (1H, s, br). MS (ES): C₂₁H₁₉CIN₄O₄ requires 426; found 427.1 (M+H⁺).

Example E19

### 3-[4-(5-{5-Chloro-6-[(1-methylethyl)oxy]-3-pyridinyl}-1,2,4-oxadiazol-3-yl)-1-methyl-1H-indol-7-yl]propanoic acid (E19)

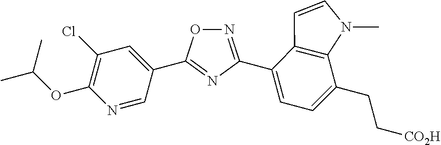

The mixture of methyl 3-[4-(5-{5-chloro-6-[(1-methylethyl)oxy]-3-pyridinyl}-1,2,4-oxadiazol-3-yl)-1-methyl-1H-indol-7-yl]propanoate (50 mg)and sodium hydroxide (44 mg) (20% solution, 0.25 mL) in methanol (5 mL) and THF (5 mL) was heated up to 60° C. for 30 min. The reaction was monitored by LCMS. After cooled to RT, the mixture was neutralized to pH=6-7 by 1N HCl. The solvent was removed under reduced pressure, to the residue was added water and extracted by ethyl acetate. After removed the solvent, the residue was purified by MDAP (dissolved in DMF) to obtain 15 mg 3-[4-(5-{5-chloro-6-[(1-methylethyl)oxy]-3-pyridinyl}-1,2,4-oxadiazol-3-yl)-1-methyl-1H-indol-7-yl]propanoic acid (E19).

δH (DMSO-d₆, 400 MHz): 1.38 (6H, d), 2.67 (2H, t), 3.41 (2H, t), 4.10 (3H, s), 5.45 (1H, m), 7.08-7.13 (2H, m), 7.46 (1H, d), 7.83 (1H, d), 8.57 (1H, d), 8.94 (1H, d), 12.28 (1H, s, br). MS (ES): C₂₂H₂₁CIN₄O₄ requires 440; found 441.2 (M+H⁺).

### EXAMPLE 20

### 3-[4-(5-{5-Chloro-6-[(1-methylethyl)oxy]-3-pyridinyl}-1,2,4-oxadiazol-3-yl)-1-ethyl-1H-indol-7-yl]propanoic acid (E20)

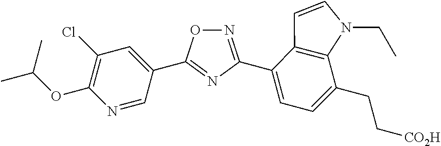

The mixture of ethyl 3-[4-(5-{5-chloro-6-[(1-methylethyl)oxy]-3-pyridinyl}-1,2,4-oxadiazol-3-yl)-1-ethyl-1H-indol-7-yl]propanoate (40 mg) and sodium hydroxide (40 mg) (20% solution, 0.2 mL) in methanol (5 mL) and THF (1 mL) was heated up to 55° C. for 30 min. The reaction was monitored by LCMS. After cooled to RT, the mixture was neutralized to pH=6-7 by 1N HCl. The solvent was removed under reduced pressure, to the residue was added water and extracted by ethyl acetate. After removed the solvent, the residue was purified by MDAP (dissolved in THF) to obtain 25 mg 3-[4-(5-{5-chloro-6-[(1-methylethyl)oxy]-3-pyridinyl}-1,2,4-oxadiazol-3-yl)-1-ethyl-1H-indol-7-yl]propanoic acid (E20).

δH (DMSO-d₆, 400 MHz): 1.38 (9H, m), 2.67 (2H, t), 3.33 (2H, t), 4.43 (2H, q), 5.45 (1H, m), 7.13-7.15 (2H, m), 7.56 (1H, d), 7.84 (1H, d), 8.57 (1H, d), 8.94 (1H, d), 12.30 (1H, s, br). MS (ES): C₂₃H₂₃CIN₄O₄ requires 454.1; found 455.2 (M+H⁺).

### EXAMPLE 21

### 4-(4-(5-{5-Chloro-6-[(1-methylethyl)oxy]-3-pyridinyl}-1,2,4-oxadiazol-3-yl)-1H-indol-7-yl]butanoic acid (E21)

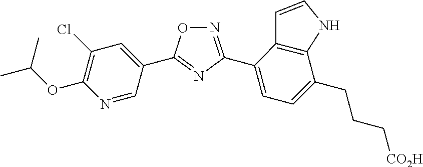

The mixture of ethyl 4-[4-(5-{5-chloro-6-[(1-methylethyl)oxy]-3-pyridinyl}-1,2,4-oxadiazol-3-yl)-1H-indol-7-yl]butanoate (55 mg) and sodium hydroxide (56 mg) (20% solution, 0.25 mL) in ethanol (10 mL) and THF (1 mL) was heated up to 60° C. for 30 min. The reaction was monitored by LCMS. After cooled to RT, the mixture was neutralized to pH=6-7 by 1N HCl. The solvent was removed under reduced pressure, to the residue was added water and extracted by ethyl acetate. After removed the solvent, the residue was purified by MDAP to obtain 14 mg 4-[4-(5-{5-chloro-6-[(1-methylethyl)oxy]-3-pyridinyl}-1,2,4-oxadiazol-3-yl)-1H-indol-7-yl]butanoic acid (E21).

δH (DMSO-d₆, 400 MHz): 1.39 (6H, d), 1.93 (2H, t), 2.29 (2H, t), 2.95 (2H, t), 5.45 (1H, m), 7.10 (2H, d), 7.54 (1H, d), 7.86 (1H, d), 8.57 (1H, s), 8.95 (1H, s), 11.51 (1H, s, br), 12.04 (1H, s, br). MS (ES): C₂₂H₂₁CIN₄O₄ requires 440.1; found 441.1 (M+H⁺).

### EXAMPLE 22

### 4-[4-(5-{5-Chloro-6-[(1-methylethyl)oxy]-3-pyridinyl}-1,2,4-oxadiazol-3-yl)-1-methyl-1H-indol-7-yl]butanoic acid (E22)

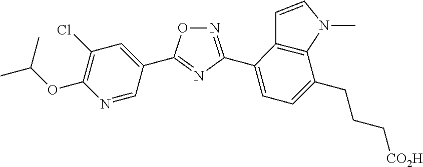

The mixture of ethyl 4-[4-(5-{5-chloro-6-[(1-methylethyl)oxy]-3-pyridinyl}-1,2,4-oxadiazol-3-yl)-1-methyl-1H-indol-7-yl]butanoate (180 mg) and sodium hydroxide (50 mg) (20% solution, 0.25 mL) in ethanol (10 mL) was heated up to reflux for 30 min. The reaction was monitored by LCMS. After cooled to RT, the mixture was neutralized to pH=6-7 by 1N HCl. The solvent was removed under reduced pressure, to the residue was added water and extracted by ethyl acetate. After removed the solvent, the residue was purified by MDAP to obtain the compound 4-[4-(5-{5-chloro-6-[(1-methylethyl)oxy]-3-pyridinyl1-1,2,4-oxadiazol-3-yl)-1-methyl-1H-indol-7-yl]butanoic acid (E22).

δH (DMSO-d₆, 400 MHz): 1.38 (6H, d), 1.89 (2H, t), 2.37 (2H, t), 3.14 (2H, t), 4.08 (3H, s), 5.44 (1H, m), 7.08 (2H, m), 7.45 (1H, d), 7.84 (1H, d), 8.56 (1H, d), 8.94 (1H, d), 12.14 (1H, s, br). MS (ES): C₂₃H₂₃CIN₄O₄ requires 454.1; found 455.2 (M+H⁺).

### EXAMPLE 23

### 4-(4-(5-{5-Chloro-6-[(1-methylethyl)oxy]-3-pyridinyl}-1,2,4-oxadiazol-3-yl)-1-ethyl-1H-indol-7-yl]butanoic acid (E23)

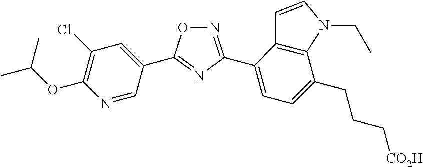

At RT, under nitrogen, potassium hydroxide (29.9 mg) was added to the solution of ethyl 4-[4-(5-{5-chloro-6-[(1-methylethyl)oxy]-3-pyridinyl}-1,2,4-oxadiazol-3-yl)-1H-indol-7-yl]butanoate (50 mg) and bromoethane (0.08 mL) in dimethyl sulfoxide (4 mL). The resulting mixture was stirred at RT for 3 hours. LCMS showed the alkylation of the nitrogen of indole cycle was completed. To this mixture was added 6 mL water and 5 mL ethanol, the resulting mixture was heated up to 65° C. stirring for 30 min. The reaction was monitored by LCMS showing the hydrolysis was completed. After neutralized the reaction mixture to pH 5-6 with concentrated HCl at 0° C., solvent was removed under reduced pressure. The residue was extracted with ethyl acetate and the combined organic layer was washed with brine, dried over sodium sulphate and evaporated to dryness. The crude product was purified by MDAP to obtain 25 mg 4-[4-(5-{5-chloro-6-[(1-methylethyl)oxy]-3-pyridinyl}-1,2,4-oxadiazol-3-yl)-1-ethyl-1H-indol-7-yl]butanoic acid (E23).

δH (CDCl₃, 400 MHz): 1.45 (9H, t), 2.09 (2H, t), 2.51 (2H, t), 3.14 (2H, t), 4.41 (2H, q), 5.49 (1H, m), 7.09 (1H, d), 7.23 (1H, d), 7.33 (1H, d), 7.94 (1H, d), 8.43 (1H, d), 8.90 (1H, d). MS (ES): C₂₄H₂₅CIN₄O₄ requires 468.1; found 469.2 (M+H⁺).

### EXAMPLE 24

### 3-[4-(5-{3-Cyano-4-[(1-methylethyl)oxy]phenyl}-1,2,4-oxadiazol-3-yl)-1H-indol-7-yl]propanoic acid (E24)

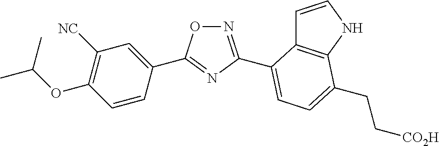

A mixture of ethyl 3-[7-(5-{3-chloro-4-[(1-methylethyl)oxy]phenyl}-1,2,4-oxadiazol-3-yl)-1H-indol-4-yl]propanoate (58 mg), sodium hydroxide (207 mg) (1 mL, 20% solution in water) and ethanol (10 mL) was heated up to 70° C. for one hour. After cooled to RT, the mixture was neutralized to pH=6-7 with 1N HCl. The mixture was evaporated under reduced pressure and extracted with ethyl acetate, the organic layer was washed with brine, dried over sodium sulphate, evaporated under reduced pressure and the residue was purified by MDAP obtaining 175 mg 3-[4-(5-{3-cyano-4-[(1-methylethyl)oxy]phenyl}-1,2,4-oxadiazol-3-yl)-1H-indol-7-yl]propanoic acid (E24).

δH (DMSO-d₆, 400 MHz): 1.37 (6H, d), 2.68 (2H, t), 3.17 (2H, t), 4.96 (1H, m), 7.09-7.13 (2H, m), 7.53 (2H, m), 7.84 (1H, d), 8.42 (1H, d), 8.52 (1H, d), 11.56 (1H, s, br), 12.19 (1H, s, br). MS (ES): C₂₃H₂₀N₄O₄ requires 416.1; found 417.2 (M+H⁺).

### EXAMPLE 25

### 3-[4-(5-{3-Cyano-4-[(1-methylethyl)oxy]phenyl}-1,2,4-oxadiazol-3-yl)-1-methyl-1H-indol-7-yl]propanoic acid (E25)

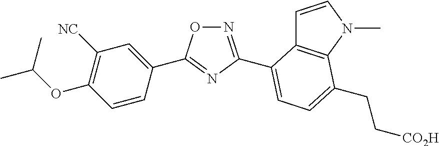

A mixture of ethyl 3-[7-(5-{3-chloro-4-[(1-methylethyl)oxy]phenyl}-1,2,4-oxadiazol-3-yl)-1H-indol-4-yl]propanoate (58 mg), sodium hydroxide (50 mg) (1 mL, 5% solution in water) and ethanol (10 mL) was heated up to 65° C. for one hour. After cooled to RT, the mixture was neutralized to pH=6-7 with 1N HCl. The mixture was evaporated under reduced pressure and extracted with ethyl acetate, the organic layer was washed with brine, dried over sodium sulphate, evaporated under reduced pressure to obtain 260 mg 3-[4-(5-{3-cyano-4-[(1-methylethyl)oxy]phenyl}-1,2,4-oxadiazol-3-yl)-1-methyl-1H-indol-7-yl]propanoic acid (E25).

δH (DMSO-d₆, 400 MHz): 1.32 (6H, d), 2.60 (2H, t), 3.35 (2H, t), 4.04 (3H, s), 4.91 (1H, m), 7.02-7.06 (2H, m), 7.39 (1H, dd), 7.48 (1H, d), 7.76 (1H, d), 8.34 (1H, d), 8.44 (1H, d), 12.33 (1H, s, br). MS (ES): C₂₄H₂₂N₄O₄ requires 430.1; found 431.2 (M+H⁺)

### EXAMPLE 26

### {(7-(5-{3-Chloro-4-[(1-methylethyl)oxy]phenyl}-1,2,4-oxadiazol-3-yl)-1-benzofuran-4-yl]oxy}acetic acid (E26)

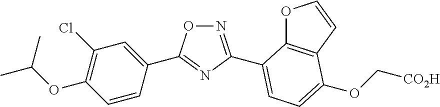

The mixture of ethyl {[7-(5-{3-chloro-4-[(1-methylethyl)oxy]phenyl}-1,2,4-oxadiazol-3-yl)-1-benzofuran-4-yl]oxy}acetate (45 mg) and sodium hydroxide (4 mg) (20% aq solution) in ethanol (10 mL) was heated up to 60° C. for 30 min. The reaction was monitored by LCMS showing the hydrolysis was completed. After neutralized the reaction mixture to pH 5-6 with concentrated HCl at 0° C., solvent was removed under reduced pressure. The residue was extracted with ethyl acetate and the combined organic layer was washed with brine, dried over sodium sulphate and evaporated to dryness. The crude product was purified by MDAP to obtain 40 mg {[7-(5-{3-chloro-4-[(1-methylethyl)oxy]phenyl}-1,2,4-oxadiazol-3-yl)-1-benzofuran-4-yl]oxy}acetic acid as white solid.

δH (DMSO-dₛ, 400 MHz): 1.36 (6H, d), 4.84-4.92 (3H, m), 6.92 (1H, d), 7.10 (1H, d), 7.44 (1H, d), 8.01 (1H, d), 8.12 (2H, m), 8.18 (1H, d). MS (ES): C₂₁H₁₇CIN₂O₆ requires 428.1; found 429.2 (M+H⁺).

### EXAMPLE 27

### 4-{[7-(5-{3-Chloro-4-[(1-methylethyl)oxy]phenyl}-1,2,4-oxadiazol-3-yl)-1-benzofuran-4-yl]oxy}butanoic acid (E27)

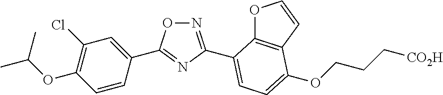

The mixture of ethyl 4-{[7-(5-{3-chloro-4-[(1-methylethyl)oxy]phenyl}-1,2,4-oxadiazol-3-yl)-1-benzofuran-4-yl]oxy}butanoate (120 mg) and sodium hydroxide (99 mg) (20% aq solution) in ethanol (4 mL) and THF (4 mL)was heated up to 50° C. for 30 min. The reaction was monitored by LCMS showing the hydrolysis was completed. After neutralized the reaction mixture to pH=5-6 with concentrated HCl at 0° C., solvent was removed under reduced pressure. The residue was extracted with ethyl acetate and the combined organic layer was washed with brine, dried over sodium sulphate and evaporated to dryness. The crude product was purified by MDAP to obtain 60 mg 4-{[7-(5-{3-chloro-4-[(1-methylethyl)oxy]phenyl}-1,2,4-oxadiazol-3-yl)-1-benzofuran-4-yl]oxy}butanoic acid (E27).

δH (DMSO-d₆, 400 MHz): 1.36 (6H, d), 2.05 (2H, t), 2.44 (2H, m), 4.24 (2H, t), 4.88 (1H, m), 7.02 (1H, d), 7.09 (1H, d), 7.44 (1H, d), 8.03 (1H, d), 8.11 (2H, m), 8.19 (1H, d), 12.18 (1H, s, br). MS (ES): C₂₃H₂₁CIN₂O₆ requires 456.1; found 457.2 (M+H⁺).

### EXAMPLE 28

### {[4-(5-{3-Chloro-4-[(1-methylethyl)oxy]phenyl}-1,2,4-oxadiazol-3-yl)-1-benzofuran-7-yl]oxy}acetic acid (E28)

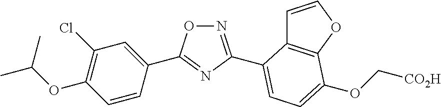

The mixture of ethyl{[4-(5-{3-chloro-4-[(1-methylethyl)oxy]phenyl}-1,2,4-oxadiazol-3-yl)-1-benzofuran-7-yl]oxy}acetate (60 mg) and sodium hydroxide (52 mg) (20% aq solution) in ethanol (10 mL) was heated up to 60° C. for 30 min. The reaction was monitored by LCMS showing the hydrolysis was completed. After neutralized the reaction mixture to pH 5-6 with concentrated HCl at 0° C., solvent was removed under reduced pressure. The residue was extracted with ethyl acetate and the combined organic layer was washed with brine, dried over sodium sulphate and evaporated to dryness. The crude product was purified by MDAP to obtain 50 mg {[4-(5-{3-chloro-4-[(1-methylethyl)oxy]phenyl}-1,2,4-oxadiazol-3-yl)-1-benzofuran-7-yl]oxy}acetic acid (E28).

δH (DMSO-d₆, 400 MHz): 1.36 (6H, d), 4.88 (1H, m), 4.99 (2H, s), 7.07 (1H, d), 7.44 (1H, d), 7.46 (1H, d), 7.98 (1H, d), 8.12 (1H, d), 8.21 (2H, m), 13.2 (1H, s, br). MS (ES): C₂₁H₁₇CIN₂O₆ requires 428.1; found 429.2 (M+H⁺).

### EXAMPLE 29

### 4-{[4-(5-{3-Chloro-4-[(1-methylethyl)oxy]phenyl}-1,2,4-oxadiazol-3-yl)-1-benzofuran-7-yl]oxy}butanoic acid (E29)

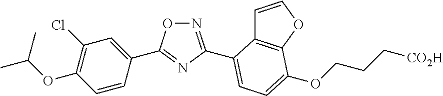

The mixture of ethyl 4-{[4-(5-{3-chloro-4-[(1-methylethyl)oxy]phenyl}-1,2,4-oxadiazol-3-yl)-1-benzofuran-7-yl]oxy}butanoate (50 mg) and sodium hydroxide (41 mg) (20% aq solution) in ethanol (10 mL) was heated up to 60° C. for 30 min. The reaction was monitored by LCMS showing the hydrolysis was completed. After neutralized the reaction mixture to pH 5-6 with concentrated HCl at 0° C., solvent was removed under reduced pressure. The residue was extracted with ethyl acetate and the combined organic layer was washed with brine, dried over sodium sulphate and evaporated to dryness. The crude product was purified by MDAP to obtain 25 mg 4-{[4-(5-{3-chloro-4-[(1-methylethyl)oxy]phenyl}-1,2,4-oxadiazol-3-yl)-1-benzofuran-7-yl]oxy}butanoic acid (E29).

δH (DMSO-d₆, 400 MHz): 1.36 (6H, d), 2.05 (2H, t), 2.45 (2H, m), 4.28 (2H, t), 4.88 (1H, m), 7.14 (1H, d), 7.44 (1H, d), 7.45 (1H, d), 8.00 (1H, d), 8.11-8.21 (3H, m), 12.20 (1H, s, br). MS (ES): C₂₃H₂₁CIN₂O₆ requires 456.1; found 457.2 (M+H⁺).

### EXAMPLE 30

### {[4-(5-{5-Chloro-6-[(1-methylethyl)oxy]-3-pyridinyl}-1,2,4-oxadiazol-3-yl)-1-benzofuran-7-yl]oxy}acetic acid (E30)

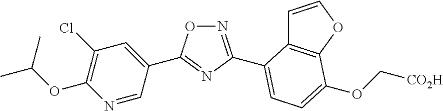

The mixture of ethyl {[4-(5-{5-chloro-6-[(1-methylethyl)oxy]-3-pyridinyl}-1,2,4-oxadiazol-3-yl)-1-benzofuran-7-yl]oxy}acetate (147 mg) and NaOH (160 mg) in ethanol (4 mL), THF (4 mL) and water (2.0 mL) was stirred at 65° C. for 2 hours. The organics was removed under vacuum and the resulting mixture was acidified with aqueous HCl (2 mL). The resulting mixture was extracted with EtOAc The organic fractions were combined and concentrated in vacuum. The resulting residue was purified by MDAP to afford {[4-(5-{5-chloro-6-[(1-methylethyl)oxy]-3-pyridinyl}-1,2,4-oxadiazol-3-yl)-1-benzofuran-7-yl]oxy}acetic acid (E30) (54 mg) as white solid.

δH (DMSO-d₆, 400 MHz): 1.38 (6H, d), 5.00 (2H, s), 5.45 (1H, m), 7.09 (1H, d), 7.50 (1H, s), 7.98 (1H, d), 8.20 (1H, s), 8.58 (1H, s), 8.95 (1H, s), 13.22 (1H, s). MS (ES): C₂₀H₁₆CIN₃O₆ requires 429; found 430.1 (M+H⁺).

### EXAMPLE 31

### 4-{(4-(5-{5-Chloro-6-[(1-methylethyl)oxy]-3-pyridinyl}-1,2,4-oxadiazol-3-yl)-1-benzofuran-7-yl]oxy}butanoic acid (E31)

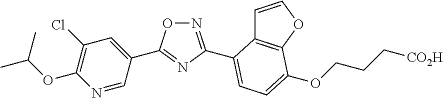

The mixture of ethyl 4-{[4-(5-{5-chloro-6-[(1-methylethyl)oxy]-3-pyridinyl}-1,2,4-oxadiazol-3-yl)-1-benzofuran-7-yl]oxy}butanoate (131 mg), NaOH (216 mg) in THF (2 mL), ethanol (2 mL) and water (1.5 mL) was stirred at 65° C. for 2 hours. The organics was removed under vacuum and the resulting solution was acidified with aqueous HCl (2 mL). The resulting mixture was extracted with EtOAc The organic fractions were combined and concentrated in vacuum. The resulting residue was purified by MDAP to afford 4-{[4-(5-{5-chloro-6-[(1-methylethyl)oxy]-3-pyridinyl}-1,2,4-oxadiazol-3-yl)-1-benzofuran-7-yl]oxy}butanoic acid (E31) (73 mg) as white solid.

δH (DMSO-d₆, 400 MHz): 1.38 (6H, d), 2.05 (2H, t), 2.45 (2H, m), 4.28 (2H, t), 5.45 (1H, m), 7.16 (1H, d), 7.48 (1H, s), 8.02 (1H, d), 8.17 (1H, s), 8.59 (1H, s), 8.96 (1H, s), 12.20 (1H, s). MS (ES): C₂₂H₂₀CIN₃O₆ requires 457; found 458.1 (M+H⁺).

### EXAMPLE 32

### 3-[4-(5-{3-Chloro-4-[(1-methylethyl)oxy]phenyl}-1,2,4-oxadiazol-3-yl)-1-benzofuran-7-yl]propanoic acid (E32)

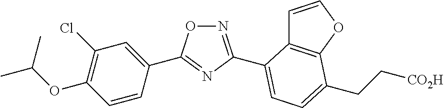

The mixture of ethyl 3-[4-(5-{3-chloro-4-[(1-methylethyl)oxy]phenyl}-1,2,4-oxadiazol-3-yl)-1-benzofuran-7-yl]propanoate (120 mg) and NaOH (80 mg) in THF (1 mL), ethanol (1 mL) and water (2 mL) was stirred at 65° C. for one hour. The organics was removed under vacuum and the resulting solution was acidified with aqueous HCl (2 mL). The resulting mixture was extracted with EtOAc The organic fractions were combined and concentrated in vacuum. The resulting residue was purified by MDAP to afford 3-[4-(5-{3-chloro-4-[(1-methylethyl)oxy]phenyl}-1,2,4-oxadiazol-3-yl)-1-benzofuran-7-yl]propanoic acid (E32) (36 mg) as white solid.

δH (DMSO-d₆, 400 MHz): 1.36 (6H, d), 2.73 (2H, t), 3.18 (2H, t), 4.90 (1H, m), 7.40 (1H, d), 7.46 (1H, d), 7.48 (1H, s), 7.99 (1H, d), 8.15 (1H, d), 8.23 (2H, m), 12.25 (1H, s). MS (ES): C₂₂H₉CIN₂O₆ requires 426; found 427.2 (M+H⁺).

### EXAMPLE 33

### 3-[4-(5-{5-Chloro-6-[(1-methylethyl)oxy]-3-pyridinyl}-1,2,4-oxadiazol-3-yl)-1-benzofuran-7-yl]propanoic acid (E33)

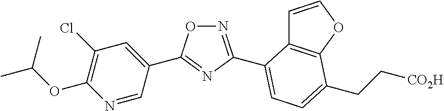

The mixture of ethyl 3-[4-(5-{5-chloro-6-[(1-methylethyl)oxy]-3-pyridinyl} 1-1,2,4-oxadiazol-3-yl)-1-benzofuran-7-yl]propanoate (100 mg), NaOH (52 mg) in ethanol (2 mL), THF (2 mL) and water (2 mL) was stirred at 65° C. for 2 hours. The organics was removed under vacuum and the resulting solution was acidified with aqueous HCl (2 mL). The resulting mixture was extracted with EtOAc The organic fractions were combined and concentrated under vacuum. The resulting residue was purified by MDAP to afford 3-[4-(5-{5-chloro-6-[(1-methylethyl)oxy]-3-pyridinyl} 1-1,2,4-oxadiazol-3-yl)-1-benzofuran-7-yl]propanoic acid (E33)(57 mg) as white solid. δH (DMSO-d₆, 400 MHz): 1.38 (6H, d), 2.73 (2H, t), 3.19 (2H, t), 5.45 (1H, m), 7.39 (1H, d), 7.50 (1H, s), 8.00 (1H, d), 8.22 (1H, s), 8.60 (1H, s), 8.96 (1H, s), 12.26 (1H, s). MS (ES): C₂₁ H ₁₈CIN₃O₅ requires 427; found 428.2 (M+H⁺).

### EXAMPLE 34

### 3-[7-(5-{3-Chloro-4-[(1-methylethyl)oxy]phenyl}-1,2,4-oxadiazol-3-yl)-1H-pyrrolo[2,3-c]pyridin-4-yl]propanoic acid (E34)

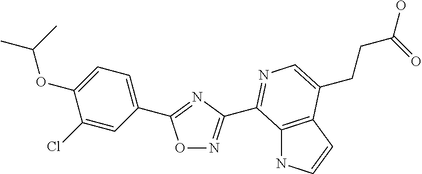

The mixture of ethyl 3-[7-(5-{3-chloro-4-[(1-methylethyl)oxy]phenyl}-1,2,4-oxadiazol-3-yl)-1H-pyrrolo[2,3-c]pyridin-4-yl]propanoate (120 mg) and NaOH (106 mg) in methanol (2 mL), THF (2 mL) and water (2 mL) was stirred at 70° C. for 1 hour. Acidified the mixture with aqueous HCl and extracted it with EA. The organic layer was concentrated in vacuum and the residue was purified by MDAP followed by lyophilization to afford 50 mg 3-[7-(5-{3-chloro-4-[(1-methylethyl)oxy]phenyl}-1,2,4-oxadiazol-3-yl)-1H-pyrrolo[2,3-c]pyridin-4-yl]propanoic acid (E34).

δH (DMSO-d₆, 400 MHz): 1.31 (6H, d), 2.67 (2H, t), 3.16 (2H, t), 4.85 (1H, m), 6.84 (1H, s), 7.40 (1H, d), 7.83 (1H, s), 8.15 (1H, d), 8.19 (1H, d), 8.44(1H, d), 11.82 (1H, s, br). MS (ES): C₂₁H₁₉CIN₄O₄ requires 426.1; found 427.2 (M+H⁺).

### EXAMPLE 35

### 3-[7-(5-{3-Cyano-4-[(1-methylethyl)oxy]phenyl}-1,2,4-oxadiazol-3-yl)-1H-pyrrolo[2,3-c]pyridin-4-yl]propanoic acid (E35)

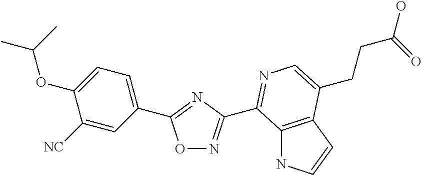

The mixture of ethyl 3-[7-(5-{3-cyano-4-[(1-methylethyl)oxy]phenyl}-1,2,4-oxadiazol-3-yl)-1H-pyrrolo[2,3-c]pyridin-4-yl]propanoate (30 mg), NaOH (27 mg) in THF (1 mL), methanol (1 mL) and water (1 mL) was stirred at 70° C. for 1 hour. Acidified the mixture with aqueous HCl and the product was purified by MDAP to obtain 5 mg 3-[7-(5-{3-cyano-4-[(1-methylethyl)oxy]phenyl}-1,2,4-oxadiazol-3-yl)-1H-pyrrolo[2,3-c]pyridin-4-yl]propanoic acid (E35).

δH (DMSO-d₆, 400 MHz): 1.33 (6H, d), 2.65 (2H, t), 3.15 (2H, t), 4.95 (1H, m), 6.79 (1H, s), 7.52 (1H, d), 7.77 (1H, d), 8.14 (1H, s), 8.51 (1H, dd), 8.81 (1H, d), 11.68 (1H, s, br). MS (ES): C₂₂H₁₉N₆O₄ requires 417.1; found 418.2 (M+H⁺).

S1P1 Tango Assay—96 Well Format

Recombinant EDG1-bla/U2OS cells (contain the human Endothelial Differentiation Gene 1 (EDG1) linked to a TEV protease site and a Gal4-VP16 transcription factor stably integrated into the Tango GPCR-bla U2OS parental cell line) were suspended in assay medium (Invitrogen Freestyle Expression Medium) at a density of 312, 500 cells/ml. Add 100 μl/well of the assay medium to the cell-free control wells (column 12) and 100 μl/well of the cell suspension to the test compound wells (row 2-8, column 1-10), the unstimulated control wells (DMSO) (column 11), and stimulated control wells (S1P) (row 1, column 1-10) in a Coming black-well, clear bottom 96-well plate. Cells were incubated at 37° C., 5% CO₂ for 44-48 h.

Add 25 μl of 5× stock solution of test compounds in assay medium with 0.5% DMSO to the test compound wells, 25p1 of 5x stock solution of agonist (S1P) in assay medium with 0.5% DMSO to the stimulated compound wells, and 25 μl of 5× stock solution of 0.5% DMSO in assay medium to the unstimulated control and cell-free control wells.

After incubation at 37° C., 5% CO₂ for 5 h, 25 μl of 6x substrate mixture (6 μl Solution A (1 mg LiveBLAzer™-FRET B/G substrate (CCF4-AM) in 912 μl DMSO) plus 60 μl Solution B plus 934 μl Solution C) was added to each well and incubate at room temperature for 2 h in dark. The plate was finally read on EnVision for two emission channels (460 nm and 530 nm).

All test compounds were dissolved in DMSO at a concentration of 10 mM and were prepared in 100% DMSO using a 1 in 5 dilution step to provide 10 point dose response curves. The dilutions were transferred to the assay plates ensuring that the DMSO concentration was constant across the plate for all assays.

Calculate the blue/green emission ratio (460 nm/530 nm) for each well, by dividing the background-subtracted Blue emission values by the background-subtracted Green emission values. The dose response curve is based on sigmoidal dose-response model. All ratio data was normalized based upon the maximum emission ratio of positive control (SIP) and minimum emission ratio of negative control (DMSO) on each plate. The intrinsic activity (IA) of each compound would be the normalized percentage of its maximum response after curve fitting.

Exemplified compounds of the invention had a pEC50>6.0.

S1P1 Tango Assay—384 Well Format

Recombinant EDG1-bla/U2OS cells (contain the human Endothelial Differentiation Gene 1 (EDG1) linked to a TEV protease site and a Gal4-VP16 transcription factor stably integrated into the Tango GPCR-bla U2OS parental cell line) were harvested from growth medium and passaged into assay medium (Invitrogen Freestyle Expression Medium). The cells were starved for 24 hours at 37° C., 5% CO₂, harvested and resuspended in assay medium at a density of ˜200,000 cells/ml.

All test compounds were dissolved in DMSO at a concentration of 10 mM and were prepared in 100% DMSO to provide 10 point dose response curves. Test compounds prepared by Bravo (Velocity11) were added to wells in columns 2-11 and 13-22; DMSO was added to wells in columns 12 and 23 as unstimulated controls and assay medium was added to wells in columns 1 and 24 as cell-free controls. An S1P1 agonist was added to wells in row 2, columns 2-11 as stimulated controls and test compounds were added to wells in row 2, columns 13-22 and rows 3-15, columns 2-11/13-22 (row 1 and 16 were empty and not used). Compounds in solution were added to the assay plate (Greiner 781090) using an Echo (Labcyte) dose-response program (50 nl/well). The unstimulated and cell-free controls were loaded with 50 nl/well pure DMSO to ensure that the DMSO concentration was constant across the plate for all assays.

50 μl of the cell suspension was added to each well in columns 2-23 of the plate (˜10,000 cells per well). 50 μl of assay medium was added to each well in the cell-free controls (columns 1 and 24). The cells were incubated overnight at 37° C/5% CO₂.

10 μl of 6× substrate mixture (LiveBLAzer™-FRET BIG substrate (CCF4-AM) Cat #K1096 from Invitrogen, Inc.) was added to each well using Bravo and the plates incubated at room temperature for 2 h in the dark. The plate was finally read on EnVision using one excitation channel (409 nm) and two emission channels (460 nm and 530 nm).

The blue/green emission ratio (460 nm/530 nm) was calculated for each well, by dividing the background-subtracted Blue emission values by the background-subtracted Green emission values. The dose response curve is based on sigmoidal dose-response model. All ratio data was normalized based upon the maximum emission ratio of positive control and minimum emission ratio of negative control (DMSO) on each plate. The intrinsic activity (IA) of each compound would be the normalized percentage of its maximum response after curve fitting.

Exemplified compounds of the invention had a pEC50>7.0.

S1P3 GeneBlazer Assay

GeneBLAzer EDG3-Ga15-NFAT-bla HEK 293T cells (contain the human Endothelial Differentiation G-protein Coupled Receptor 3 (EDG3) and a beta-lactamase reporter gene under control of a NFAT response element and a promiscuous G Protein, Ga15, stably integrated into the GeneBLAzer Ga15-NFAT-bla HEK 293T cell line) were suspended in assay medium (99% DMEM, 1% Dialyzed FBS, 0.1 mM NEAA, 25 mM HEPES (pH 7.3), 100 U/ml penicillin, 100 μg/ml streptomycin) at a density of 312, 500 cells/ml. Add 100 μl/well of the assay medium to the cell-free control wells (column 12) and 100 μl/well of the cell suspension to the test compound wells (row 2-8, column 1-10), the unstimulated control wells (DMSO) (column 11), and stimulated control wells (S1P) (row 1, column 1-10) in a Coming black-well, clear bottom 96-well plate. Cells were incubated at 37° C., 5% CO2 for 24 h.

Add 25 μl of 5× stock solution of test compounds in assay medium with 0.5% DMSO to the test compound wells, 25 μl of 5× stock solution of agonist (S1P) in assay medium with 0.5% DMSO to the stimulated compound wells, and 25 μl of 5× stock solution of 0.5% DMSO in assay medium to the unstimulated control and cell-free Control wells.

After incubation at 37° C., 5% CO2 for 5 h, 25 μl of 6× substrate mixture (6 μl Solution A (1 mg LiveBLAzer™-FRET B/G Substrate (CCF4-AM) in 912 μl DMSO) plus 60 μl Solution B plus 934 μl Solution C) was added to each well and incubate at room temperature for 2 h in dark. The plate was finally read on EnVision for two emission channels (460 nm and 530 nm).

All test compounds were dissolved in DMSO at a concentration of 10 mM and were prepared in 100% DMSO using a 1 in 5 dilution step to provide 10 point dose response curves. The dilutions were transferred to the assay plates ensuring that the DMSO concentration was constant across the plate for all assays.

Calculate the blue/green emission ratio (460 nm/530 nm) for each well, by dividing the background-subtracted Blue emission values by the background-subtracted green emission values. The dose response curve is based on sigmoidal dose-response model. All ratio data was normalized based upon the maximum emission ratio of positive control (SIP) and minimum emission ratio of negative control (DMSO) on each plate. The intrinsic activity (IA) of each compound would be the normalized percentage of its maximum response after curve fitting.

Exemplified compounds of the invention tested in at least one of the above assays had a pEC50<6.

1. A compound of formula (I) or a salt thereof: 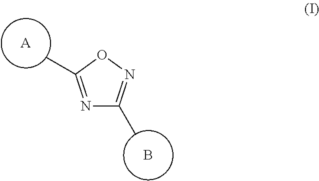
wherein
A is an aromatic ring selected from: 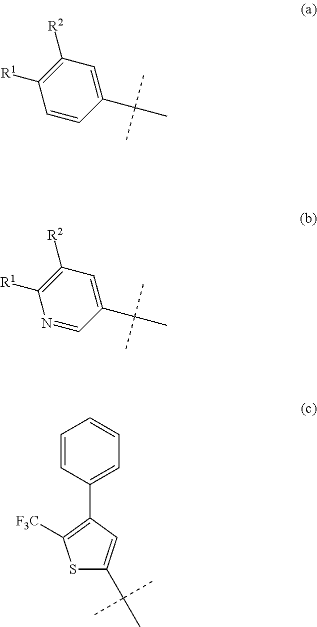
R¹ is C₍₁₋₆₎alkoxy or C₍₁₋₆₎alkyl;
R² is halogen, cyano or CF₃;
B is a bicyclic ring selected from: 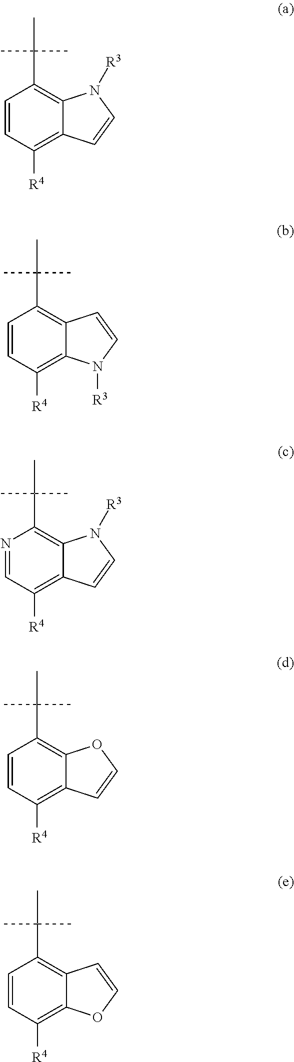
R³ is hydrogen or C₍₁₋₃₎alkyl; and
R⁴ is C₍₀₋₆₎alkyl-COOH optionally interrupted by a cyclopropyl, piperidinyl, azetidinyl, pyrrolidinyl, optionally interrupted by N or O and optionally substituted by O, cyclopropyl, halogen or methyl.

2. A compound of formula (I) or a salt thereof, wherein:
A is (a) or (b)
R¹ is C₍₁₋₃₎alkoxy;
R² is chloro or cyano;
R³ is hydrogen, methyl or ethyl; and
when B is (e), (f) or (g), R⁴ is C₍₀₋₃₎alkyl-COOH or when B is (h) or (i), R⁴ is C(₂)alkyl-COOH or O—C₍₁₋₃₎alkyl-COOH.

3. A compound selected from the group consisting of:
3-[7-(5-{3-Chloro-4-[(1-methylethyl)oxy]phenyl}-1,2,4-oxadiazol-3-yl)-1H-indol-4-yl]propanoic acid;
3-[7-(5-{3-Chloro-4-[(1-methylethyl)oxy]phenyl}-1,2,4-oxadiazol-3-yl)-1-methyl-1H-indol-4-yl]propanoic acid;
3-[7-(5-{3-Chloro-4-[(1-methylethyl)oxy]phenyl}-1,2,4-oxadiazol-3-yl)-1-ethyl-1H-indol-4-yl]propanoic acid;
3-[7-(5-{5-Chloro-6-[(1-methylethyl)oxy]-3-pyridinyl}-1,2,4-oxadiazol-3-yl)-1H-indol-4-yl]propanoic acid;
3-[7-(5-{5-Chloro-6-[(1-methylethyl)oxy]-3-pyridinyl}-1,2,4-oxadiazol-3-yl)-1-methyl-1H-indol-4-yl]propanoic acid;
3-[7-(5-{3-Cyano-4-[(1-methylethyl)oxy]phenyl}-1,2,4-oxadiazol-3-yl)-1H-indol-4-yl]propanoic acid;
3-[7-(5-{3-Cyano-4-[(1-methylethyl)oxy]phenyl}-1,2,4-oxadiazol-3-yl)-1-methyl-1H-indol-4-yl]propanoic acid;
4-[7-(5-{3-Chloro-4-[(1-methylethyl)oxy]phenyl}-1,2,4-oxadiazol-3-yl)-1H-indol-4-yl]butanoic acid;
4-[7-(5-{3-Chloro-4-[(1-methylethyl)oxy]phenyl}-1,2,4-oxadiazol-3-yl)-1-methyl-1H-indol-4-yl]butanoic acid;
4-[7-(5-{5-Chloro-6-[(1-methylethyl)oxy]-3-pyridinyl}1-1,2,4-oxadiazol-3-yl)-1H-indol-4-yl]butanoic acid;
4-[7-(5-{5-Chloro-6-[(1-methylethyl)oxy]-3-pyridinyl}1-1,2,4-oxadiazol-3-yl)-1-methyl-1H-indol-4-yl]butanoic acid;
3-[4-(5-{3-Chloro-4-[(1-methylethyl)oxy]phenyl}-1,2,4-oxadiazol-3-yl)-1H-indol-7-yl]propanoic acid;
3-[4-(5-{3-Chloro-4-[(1-methylethyl)oxy]phenyl}-1,2,4-oxadiazol-3-yl)-1-methyl-1H-indol-7-yl]propanoic acid; 3-[4-(5-{3-Chloro-4-[(1-methylethyl)oxy]phenyl}-1,2,4-oxadiazol-3-yl)-1-ethyl-1H-indol-7-yl]propanoic acid;
4-[4-(5-{3-Chloro-4-[(1-methylethyl)oxy]phenyl}-1,2,4-oxadiazol-3-yl)-1H-indol-7-yl]butanoic acid;
4-[4-(5-{3-Chloro-4-[(1-methylethyl)oxy]phenyl}-1,2,4-oxadiazol-3-yl)-1-methyl-1H-indol-7-yl]butanoic acid;
4-[4-(5-{3-Chloro-4-[(1-methylethyl)oxy]phenyl}-1,2,4-oxadiazol-3-yl)-1-ethyl-1H-indol-7-yl]butanoic acid;
3-[4-(5-{5-Chloro-6-[(1-methylethyl)oxy]-3-pyridinyl}1-1,2,4-oxadiazol-3-yl)-1H-indol-7-yl]propanoic acid;
3-[4-(5-{5-Chloro-6-[(1-methylethyl)oxy]-3-pyridinyl}1-1,2,4-oxadiazol-3-yl)-1-methyl-1H-indol-7-yl]propanoic acid;
3-[4-(5-{5-Chloro-6-[(1-methylethyl)oxy]-3-pyridinyl}1-1,2,4-oxadiazol-3-yl)-1-ethyl-1H-indol-7-yl]propanoic acid;
4-[4-(5-{5-Chloro-6-[(1-methylethyl)oxy]-3-pyridinyl}1-1,2,4-oxadiazol-3-yl)-1H-indol-7-yl]butanoic acid;
4-[4-(5-{5-Chloro-6-[(1-methylethyl)oxy]-3-pyridinyl}1-1,2,4-oxadiazol-3-yl)-1-methyl-1H-indol-7-yl]butanoic acid;
4-[4-(5-{5-Chloro-6-[(1-methylethyl)oxy]-3-pyridinyl}1-1,2,4-oxadiazol-3-yl)-1-ethyl-1H-indol-7-yl]butanoic acid;
3-[4-(5-{3-Cyano-4-[(1-methylethyl)oxy]phenyl}-1,2,4-oxadiazol-3-yl)-1H-indol-7-yl]propanoic acid;
3-[4-(5-{3-Cyano-4-[(1-methylethyl)oxy]phenyl}-1,2,4-oxadiazol-3-yl)-1-methyl-1H-indol-7-yl]propanoic acid;
{[7-(5-{3-Chloro-4-[(1-methylethyl)oxy]phenyl}-1,2,4-oxadiazol-3-yl)-1-benzofuran-4-yl]oxy}acetic acid;
4-{[7-(5-{3-Chloro-4-[(1-methylethyl)oxy]phenyl}-1,2,4-oxadiazol-3-yl)-1-benzofuran-4-yl]oxy}butanoic acid;
{[4-(5-{3-Chloro-4-[(1-methylethyl)oxy]phenyl}-1,2,4-oxadiazol-3-yl)-1-benzofuran-7-yl]oxy}acetic acid;
4-{[4-(5-{3-Chloro-4-[(1-methylethyl)oxy]phenyl}-1,2,4-oxadiazol-3-yl)-1-benzofuran-7-yl]oxy}butanoic acid;
{[4-(5-{5-Chloro-6-[(1-methylethyl)oxy]-3-pyridinyl}-1,2,4-oxadiazol-3-yl)-1-benzofuran-7-yl]oxy}acetic acid
4-{[4-(5-{5-Chloro-6-[(1-methylethyl)oxy]-3-pyridinyl}-1,2,4-oxadiazol-3-yl)-1-benzofuran-7-yl]oxy}butanoic acid;
3-[4-(5-{3-Chloro-4-[(1-methylethyl)oxy]phenyl}-1,2,4-oxadiazol-3-yl)-1-benzofuran-7-yl]propanoic acid;
3-[4-(5-{5-Chloro-6-[(1-methylethyl)oxy]-3-pyridinyl}-1,2,4-oxadiazol-3-yl)-1-benzofuran-7-yl]propanoic acid;
3-[7-(5-{3-Chloro-4-[(1-methylethyl)oxy]phenyl}-1,2,4-oxadiazol-3-yl)-1H-pyrrolo[2,3-c]pyridin-4-yl]propanoic acid; and
3-[7-(5-{3-Cyano-4-[(1-methylethyl)oxy]phenyl}-1,2,4-oxadiazol-3-yl)-1H-pyrrolo[2,3-c]pyridin-4-yl]propanoic acid,
and salts thereof.

4. A method of treating a subject with a condition or disorder mediated by a S1P1 receptor comprising administering to a subject with said condition or disorder a compound of formula (1) according to claim 1 or a pharmaceutically acceptable salt thereof.

5. A method according to claim 4, wherein the condition or disorder is multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes.

6. A method according to claim 5, wherein the condition is multiple sclerosis.

7. (canceled)

8. (canceled)

9. (canceled)

10. A pharmaceutical composition comprising a compound of formula (I) or a pharmaceutically acceptable salt thereof according to claim 1.

11. (canceled)

12. (canceled)
